(19) United States
(12) Patent Application Publication
(10) Pub. No.: US 2020/0276101 A1
(43) Pub. Date: Sep. 3, 2020

(54) SEMI-PERMANENT TATTOOS

(71) Applicant: inkbox ink incorporated, Toronto (CA)

(72) Inventors: Tyler Handley, Toronto (CA); Betty Yu, Sunnyvale, CA (US); Sunny Deng, Toronto (CA); Jobey Chua, Toronto (CA); Nazanin Khalili, Toronto (CA); Motaz Adam, Toronto (CA); Hanna Jiamei Zhang, Toronto (CA); Christopher Caputo, Toronto (CA)

(21) Appl. No.: 16/785,549

(22) Filed: Feb. 7, 2020

Related U.S. Application Data
(60) Provisional application No. 62/802,688, filed on Feb. 7, 2019.
(60) Provisional application No. 62/863,560, filed on Jun. 19, 2019.

(51) Int. Cl. A61K 8/49; A61Q 1/02; A61K 8/60; A61K 8/73; A61K 8/46; A61K 8/43; A61K 8/02

(52) U.S. Cl. CPC A61K 8/498; A61Q 1/025; A61K 8/60; A61K 8/731; A61K 8/466; A61K 2800/432; A61K 8/0208; A61K 2800/88; A61K 2800/524; A61K 2800/10; A61K 8/43

(57) ABSTRACT

The present disclosure relates to semi-permanent tattoos, and more particularly to genipin-based ink compositions as well as layered adhesive articles and methods for application of the genipin-based, semi-permanent tattoos. In some embodiments, the genipin-based ink composition comprises: purified genipin present in an amount of about 5% to about 10% w/w of the composition; one or more volatile solvents present in an amount of about 70% to about 85% w/w of the composition; one or more semi-volatile semi-permanent colorant solubilizers present in an amount of about 10% to about 20% w/w of the composition; and one or more surface-active agents present in an amount of about 0.25% to about 5% w/w of the composition.

### CROSS-REFERENCE TO RELATED APPLICATIONS

This application claims the benefit of U.S. Provisional Application No. 62/802,688, filed on Feb. 7, 2019, and U.S. Provisional Application No. 62/863,560, filed on Jun. 19, 2019. The disclosures of the prior applications are incorporated by reference in their entirety.

### TECHNICAL FIELD

This invention relates to semi-permanent tattoos, and more particularly to genipin-based ink compositions as well as layered adhesive articles and methods for application of genipin-based, semi-permanent tattoos.

### BACKGROUND

Temporary or semi-permanent tattoo or body inks have been used throughout human history to decorate the body. Generally, temporary or semi-permanent tattoos are transferred to the skin through the direct exposure of the skin to tattoo ink over a designated incubation period. The quality of the transferred image and its duration on the skin can depend on the ink distribution profile in the outer layer of the skin called the stratum corneum. Current manufacturing processes for pre-fabricated tattoo designs include the utilization of flexographic or gravure printing, stencils, and inkjet printers. However, due to the barrier properties of the stratum corneum, many of the current methods require moderate oversight by the end user during the tattoo application process to ensure adequate ink delivery to the skin such that the tattoo ink develops properly.

### SUMMARY

Provided herein are ink compositions comprising: one or more semi-permanent colorants; one or more volatile solvents; and one or more semi-volatile semi-permanent colorant solubilizers.

In some embodiments, the ink composition has a viscosity from about 1 to about 20 centipoise. In some embodiments, the ink composition has a viscosity from about 1 to about 7 centipoise. In some embodiments, the ink composition is configured to have a viscosity from about 1 to about 7 centipoise. In some embodiments, the ink composition has a viscosity from about 3 to about 7 centipoise. In some embodiments, the ink composition is configured to have a viscosity from about 3 to about 7 centipoise. In some embodiments, the ink composition is configured to have a surface tension from about 20 to about 35 dynes/cm. In some embodiments, the ink composition has a surface tension from about 20 to about 35 dynes/cm.

In some embodiments, semi-permanent colorant is present in an amount of about 0.1% to about 20% w/w of the composition. In some embodiments, the one or more semi-permanent colorants are present in an amount of about 5% to about 15% w/w of the composition. In some embodiments, the one or more semi-permanent colorants are present in an amount of about 5% to about 10% w/w of the composition. In some embodiments, the one or more semi-permanent colorants are present in an amount of about 7% to about 11% w/w of the composition. In some embodiments, the one or more semi-permanent colorants are present in an amount of about 6% to about 8% w/w of the composition. In some embodiments, the one or more semi-permanent colorants are present in an amount of about 9% w/w of the composition. In some embodiments, the one or more semi-permanent colorants are present in an amount of about 7% w/w of the composition.

In some embodiments, the one or more semi-permanent colorants is colorant precursor such as genipin. In some embodiments, the genipin is purified genipin.

In some embodiments, the one or more volatile solvents are present in an amount of about 0.1% to about 95% w/w of the composition. In some embodiments, the one or more volatile solvents are present in an amount of about 60% to about 90% w/w of the composition. In some embodiments, the one or more volatile solvents are present in an amount of about 60% to about 80% w/w of the composition. In some embodiments, the one or more volatile solvents are present in an amount of about 65% to about 85% w/w of the composition. In some embodiments, the one or more volatile solvents are present in an amount of about 70% to about 80% w/w of the composition. In some embodiments, the one or more volatile solvents are present in an amount of about 50% to about 75% w/w of the composition. In some embodiments, the one or more volatile solvents are present in an amount of about 65% to about 75% w/w of the composition. In some embodiments, the one or more volatile solvents are present in an amount of about 65% to about 70% w/w of the composition. In some embodiments, the one or more volatile solvents are present in an amount of about 70% to about 75% w/w of the composition. In some embodiments, the one or more volatile solvents are present in an amount of about 75% to about 80% w/w of the composition. In some embodiments, the one or more volatile solvents are present in an amount of about 77% w/w of the composition. In some embodiments, the one or more volatile solvents are selected from the group consisting of: water, methanol, ethanol, isopropanol, and a combination of two or more thereof.

In some embodiments, the one or more volatile solvents are a combination of water and ethanol. In some embodiments, the water is present in an amount of about 40% to about 55% w/w of the composition and the ethanol is present in an amount of about 15% to about 30% w/w of the composition. In some embodiments, the water is present in an amount of about 45% to about 50% w/w of the composition and the ethanol is present in an amount of about 20% to about 27% w/w of the composition. In some embodiments, the water is present in an amount of about 25% to about 35% w/w of the composition and the ethanol is present in an amount of about 35% to about 50% w/w of the composition. In some embodiments, the water is present in an amount of about 30% w/w of the composition and the ethanol is present in an amount of about 43% w/w of the composition.

In some embodiments, the one or more semi-volatile semi-permanent colorant solubilizers are present in an amount of about 0.1% to about 95% w/w of the composition. In some embodiments, the one or more semi-volatile semi-permanent colorant solubilizers are present in an amount of about 0.1% to about 25% w/w of the composition. In some embodiments, the one or more semi-volatile semi-permanent colorant solubilizers are present in an amount of about 15% to about 25% w/w of the composition. In some embodiments, the one or more semi-volatile semi-permanent colorant solubilizers are present in an amount of about 10% to about 20% w/w of the composition. In some embodiments, the one or more semi-volatile semi-permanent colorant solubilizers are present in an amount of about 20% to about 25% w/w of the composition. In some embodiments, the one or more semi-volatile semi-permanent colorant solubilizers are present in an amount of about 15% w/w of the composition. In some embodiments, the one or more semi-volatile semi-permanent colorant solubilizers are selected from the group consisting of: a polyethylene glycol, an alkyl glycol, an alkylene glycol ether, and a combination of two or more thereof. In some embodiments, the alkyl glycol is diethylene glycol monoethyl ether. In some embodiments, the one or more semi-volatile semi-permanent colorant solubilizers is diethylene glycol monoethyl ether.

In some embodiments, the composition further comprises one or more surface-active agents. In some embodiments, the one or more surface-active agents are present in an amount of about 0.1% to about 5% w/w of the composition. In some embodiments, the one or more surface-active agents are present in an amount of about 1% to about 3% w/w of the composition. In some embodiments, the one or more surface-active agents are present in an amount of about 2% w/w of the composition. In some embodiments, the one or more surface-active agents are selected from the group consisting of: an alkylbenzene sulfonate, an alkyl sulfate, an alkyl ether sulfate, an alkyl alcohol, a soap, an ethoxylate, a lignosulfonate, and a triglyceride. In some embodiments, the one or more surface-active agents are selected from the group consisting of: sodium dodecyl benzenesulfonate, lauryl sulfate, di-alkyl sulfosuccinate, dimethyl ether of tetradecyl phosphonic, lauryl mono-ethanol, glycerol diester (diglyceride), dodecyl betaine, abietic acid, polyethoxylated octyl phenol, 1,2-hexanediol, and sorbitan monoester. In some embodiments, the one or more surface-active agents is 1,2-hexanediol.

In some embodiments, the composition further comprises one or more matrix components. In some embodiments, the composition further comprises one or more matrix components. In some embodiments, the one or more matrix components are present in an amount of about 0.1% to about 10% w/w of the composition. In some embodiments, the one or more matrix components are present in an amount of about 0.1% to about 3% w/w of the composition. In some embodiments, the one or more matrix components are present in an amount of about 2% w/w of the composition. In some embodiments, the one or more matrix components are selected from the group consisting of: a sugar, a sugar alcohol, and a polymer. In some embodiments, the sugar is selected from the group consisting of: trehalose, glucose, fructose, galactose, ribose, and xylose, trehalose, sucrose, lactotrehalose, galactotrehalose, 6-azidotrehalose, maltose, lactose, lactulose, cellobiose, chitobiose, a starch, glycogen, a cellulose, and a chitin. In some embodiments, the sugar is trehalose. In some embodiments, the one or more matrix components is trehalose. In some embodiments, the one or more matrix components is hydroxypropylmethylcellulose. In some embodiments, the composition further comprises one or more skin penetration enhancers. In some embodiments, the one or more skin penetration enhancers are present in an amount of about 0.1% to about 50% w/w of the composition. In some embodiments, the one or more skin penetration enhancers are present in an amount of about 0.5% to about 5% w/w of the composition. In some embodiments, the one or more skin penetration enhancers are selected from the group consisting of: an alcohol, an amide, an ester, an ether alcohol, a fatty acid, a glycol, a pyrrolidone, a sulphoxide, a surfactant, and a terpene. In some embodiments, the one or more skin penetration enhancers are selected from the group consisting of: ethanol, isopropanol, decanol, hexanol, lauryl alcohol, myristyl alcohol, octanol, octyl dodecanol, oleyl alcohol, azone, ethyl acetate, octyl salicylate, padimate O, ethyl oleate, glyceryl monoleate, glyceryl monocaprate, glyceryl tricaprylate, isopropyl myristate, isopropyl palmitate, propylene glycol monolaurate, propylene glycol monocaprylate, diethylene glycol monoethyl ether (TRANSCUTOL®), lauric acid, linoleic acid, linolenic acid, myristic acid, oleic acid, palmitic acid, stearic acid, isostearic acid, dipropylene glycol, propylene glycol, 1,2-butylene glycol, 1,3-butylene glycol, n-methyl-2-pyrrolidone, 2-pyrrolidone, decylmethyl sulphoxide, dimethyl sulphoxide, sodium lauryl sulphate, alkyl dimethylbenzyl ammonium halides, alkyl trimethyl ammonium halides, alkyl pyridinium halides, brij 36t, polyethylene glycol sorbitan monooleate (e.g., TWEEN® 80), eugenol, D-limonene, menthol, menthone, farnesol, and neridol.

In some embodiments, the composition further comprises one or more temporary colorants. In some embodiments, the one or more temporary colorants are present in an amount of about 0.1% to about 1% w/w of the composition. In some embodiments, the one or more temporary colorant are present in an amount of about 0.3% w/w of the composition. In some embodiments, the one or more temporary colorants are selected from the group consisting of: iron oxide black (Fe₃O₄), iron oxide (FeO), carbon, logwood, ochre, cinnabar (HgS), cadmium red (CdSe), iron (III) oxide (Fe₂O₃), naphthol-AS pigment, disazodiarylide, disazopyrazolone, cadmium seleno-sulfide, cadmium yellow (CdS, CdZnS), curcuma yellow, chrome yellow (PbCrO₄), disazodiarylide, chromium oxide (Cr₂O₃), malachite [Cu₂(CO₃)(OH)₂], a ferrocyanide, a ferricyanide, lead chromate, monoazo pigment, Cu/Al phthalocyanine, Cu phthalocyanine, azure blue, cobalt blue, Cu-phthalocyanine, manganese violet (manganese ammonium pyrophosphate), an aluminum salt, quinacridone, dioxazine/carbazole, lead white (lead carbonate), titanium dioxide (TiO₂), barium sulfate (BaSO₄), zinc oxide, anthraquinone dyes and derivatives, annatto, caramel, ß-carotene, bismuth citrate, disodium EDTA copper, potassium sodium copper chlorophyllin, dihydroxyacetone, bismuth oxychloride, guaiazulene, henna, ferric ammonium ferrocyanide, ferric ferrocyanide, chromium hydroxide green, chromium oxide greens, guanine, lead acetate, pyrophillite, mica, silver, titanium dioxide, aluminum powder, bronze powder, copper powder, ultramarines, manganese violet, zinc oxide, luminescent zinc sulfide, D&C Black Nos. 2 and 3, FD&C Blue No. 1 (e.g., acid blue 9), D&C Blue No. 4, D&C Brown No. 1, FD&C Green No. 3, D&C Green Nos. 5, 6, and 8, D&C Orange Nos. 4, 5, 10 and 11, FD&C Red Nos. 4, D&C Red Nos. 6, 7, 17, 21, 22, 27, 28, 30, 32, 33, 34, 36 and 40, D&C Violet No. 2, Ext. D&C Violet No. 2, FD&C Yellow Nos. 5 and 6, D&C Yellow Nos. 7, 8, 10 and 11, and Ext. D&C Yellow No. 7. In some embodiments, the temporary colorant does not contain an amine group. In some embodiments, the temporary colorant is acid blue 9.

In some embodiments, the ink composition has a pH of about 3 to about 8. In some embodiments, the ink composition has a pH of about 4 to about 7. In some embodiments, the ink composition has a pH of about 4.5 to about 5. In some embodiments, the ink composition has a pH of about 4.8 to about 5. In some embodiments, the ink composition is has a pH of about 4.9. In some embodiments, the ink composition is has a pH of about 4.6.

In some embodiments, the ink composition is configured to have a pH of about 3 to about 8. In some embodiments, the ink composition is configured to have a pH of about 4 to about 7. In some embodiments, the ink composition is configured to have a pH of about 4.8 to about 5. In some embodiments, the ink composition is configured to have a pH of about 4.9.

In some embodiments, the ink composition further comprises one or more preservatives. In some embodiments, the one or more preservatives are present in an amount of about 0.5% to about 1.5% w/w of the composition. In some embodiments, the one or more preservatives are present in an amount of about 1% w/w of the composition. In some embodiments, the one or more preservatives are selected form the group consisting of: ascorbic acid, an ascorbate, a palmitate, citric acid, a benzoate, a benzoic acid, a propionate, propionic acid, a sorbate, sorbic acid, a salicylic acid, a salicylate, hexa-2,4-dienoic acid, a hexa-2,4-dienoate, formaldehyde, a formaldehyde releaser, formic acid and its salts, 3-acetyl-6-methylpyran-2,4-(3H)-dione and its salts, 3,3′-dibromo-4,4′-hexamethylenedioxydibenzamidine and its salts, thiomersal, phenylmercuric salts, undec-10-enoic acid and its salts, 1,3-bis (2-ethylhexyl) hexahydro-5-methyl-5-pyrimidine, 5-bromo-5-nitro-1,3-dioxane, bronopol, 2,4-dichlorobenzyl alcohol, 1-(4-chlorophenyl)-3-(3,4-dichlorophenyl) urea, chlorocresol, chloroxylenol, 5-chloro-2-(2,4-dichlorophenoxy) phenol, N,N″-methylenebis[N′[3-(hydroxymethyl)-2,5-dioxoimidazolidin-4-yl]urea], polyaminopropyl biguanide, methenamine, quaternium-15, climbazole, DMDM hydantoin, benzyl alcohol, 1-Hydroxy-4-methyl-6-(2,4,4-trimethylpentyl)-2-pyridon, piroctone olamine, bromochlorophene, o-cymen-5-ol, chlorophene, chloroacetaminde, methylchloroisothiazolinone, methylisothiazolinone, phenoxyisopropanol, chlorhexidine, chlorhexidine diacetate, chlorhexidine digluconate, chlorhexidine dihydrochloride, dimethyl oxazolidine, behentrimonium chloride, cetri-monium bromide, cetrimonium chloride, laurtrimonium bromide, laurtrimonium chloride, steartrimonium bromide, steartrimonium chloride, diazolidinyl urea, hexamidine, hexamidine diisethionate, hexamidine paraben, glutaral, 7-ethylbicyclooxazolidine, chlorphenesin, sodium hydroxymethylglycinate, silver chloride, benzethonium chloride, benzalkonium chloride, benza-lkonium bromide, benzalkonium saccharinate, benzylhemiformal, iodopropynyl butylcarbamate, biphenyl-2-ol and its salts, pyrithionine zinc, an erythorbate, a nitrite, ethylenediaminetetraacetic acid (EDTA), butylated hydroxyanisole (BHA), butylated hydroxytoluene (BHT), capryllic acid, dilauryl thiodipropionate, erythorbic acid, gum guaiac, methylparaben, a sulfite, a bisulfite, a metabisulfite, propyl gallatepy, propylparaben, stannous chloride, sulfur dioxide, thiodipropionic acid, an isothiazoline, a paraben, phenoxyethanol, ethylhexylglycerin, a glycols, and a tocopherol. In some embodiments, the one or more preservatives are phenoxyethanol and ethylhexylglycerin.

Also provided herein are ink compositions comprising: purified genipin present in an amount of about 5% to about 10% w/w of the composition; one or more volatile solvents present in an amount of about 70% to about 85% w/w of the composition, wherein the one or more semi-volatile solvents are water and ethanol; one or more semi-volatile semi-permanent colorant solubilizers present in an amount of about 10% to about 20% w/w of the composition, wherein the one or more semi-volatile semi-permanent colorant solubilizers is diethylene glycol monoethyl ether; one or more surface-active agents present in an amount of about 0.25% to about 5% w/w of the composition; and one or more matrix components present in an amount of about 0.1% to about 5% w/w of the composition.

Also provided herein are ink compositions comprising: purified genipin present in an amount of about 7% w/w of the composition; one or more volatile solvents present in an amount of about 65% to about 70% w/w of the composition, wherein the one or more semi-volatile solvents are water and ethanol; one or more semi-volatile semi-permanent colorant solubilizers present in an amount of about 10% to about 20% w/w of the composition, wherein the one or more semi-volatile semi-permanent colorant solubilizers comprise diethylene glycol monoethyl ether; one or more surface-active agents present in an amount of about 1% to about 3% w/w of the composition; and one or more matrix components present in an amount of about 1% to about 3% w/w of the composition.

Also provided herein are ink compositions comprising: purified genipin present in an amount of about 7% w/w of the composition; one or more volatile solvents present in an amount of about 77% w/w of the composition, wherein the one or more semi-volatile solvents are water and ethanol; one or more semi-volatile semi-permanent colorant solubilizers present in an amount of about 15% w/w of the composition, wherein the one or more semi-volatile semi-permanent colorant solubilizers comprise diethylene glycol monoethyl ether; one or more surface-active agents present in an amount of about 2% w/w of the composition; and one or more matrix components present in an amount of about 2% w/w of the composition.

In some embodiments, the water is present in an amount of about 25% to about 35% w/w of the composition and the ethanol is present in an amount of about 40% to about 45% w/w of the composition. In some embodiments, the water is present in an amount of about 30% w/w of the composition and the ethanol is present in an amount of about 43% w/w of the composition.

In some embodiments, the one or more surface active agents is diethylene glycol monoethyl ether. In some embodiments, the one or more matrix components is trehalose.

In some embodiments, the composition further comprises one or more preservatives. In some embodiments, the one or more preservatives are present in an amount of about 0.5% to about 1.5% w/w of the composition. In some embodiments, the one or more preservatives are present in an amount of about 1% w/w of the composition. In some embodiments, the one or more preservatives are phenoxyethanol and ethylhexylglycerin.

In some embodiments, the composition has a viscosity from about 1 to about 7 centipoise. In some embodiments, the composition has a viscosity from about 3 to about 7 centipoise. In some embodiments, the composition has a surface tension from about 20 to about 35 dynes/cm.

In some embodiments, the composition is an inkjet printer ink composition.

Also provided herein are inkjet printer ink compositions comprising: purified genipin; one or more volatile solvents; and one or more semi-volatile semi-permanent colorant solubilizers. In some embodiments, the composition is configured to have a viscosity from about 1 to about 7 centipoise. In some embodiments, the composition is configured to have a viscosity from about 3 to about 7 centipoise. In some embodiments, the composition is configured to have a surface tension from about 20 to about 35 dynes/cm. In some embodiments, the composition further comprises one or more skin penetration enhancers. In some embodiments, composition further comprises one or more temporary colorants.

In some embodiments, the inkjet printer ink composition is configured to have a pH of about 3 to about 8. In some embodiments, the composition is configured to have a pH of about 4 to about 7. In some embodiments, the composition is configured to have a pH of about 4.8 to about 5. In some embodiments, the composition is configured to have a pH of about 4.9.

Also provided herein is an inkjet printer ink composition comprising: purified genipin present in an amount of about 7% to about 11% w/w of the composition; one or more volatile solvents present in an amount of about 60% to about 80% w/w of the composition, wherein the one or more semi-volatile solvents are water and ethanol; one or more semi-volatile semi-permanent colorant solubilizers present in an amount of about 10% to about 30% w/w of the composition, wherein the one or more semi-volatile semi-permanent colorant solubilizers is diethylene glycol monoethyl ether; one or more surface-active agents present in an amount of about 0.25% to about 5% w/w of the composition; and one or more matrix components present in an amount of about 0.1% to about 5% w/w of the composition.

In some embodiments, the one or more surface-active agents is PEG 400. In some embodiments, the one or more matrix components is trehalose.

In some embodiments, the inkjet printer ink composition further comprises one or more temporary colorants. In some embodiments, the temporary colorant is present in an amount of about 0.05% to about 0.25% w/w of the composition. In some embodiments, the one or more temporary colorants is FD&C Blue No. 1. Also provided herein is an inkjet printer ink composition comprising: purified genipin present in an amount of about 7% to about 11% w/w of the composition; one or more volatile solvents present in an amount of about 65% to about 75% w/w of the composition, wherein the one or more semi-volatile solvents are water and ethanol; one or more semi-volatile semi-permanent colorant solubilizers present in an amount of about 15% to about 25% w/w of the composition, wherein the one or more semi-volatile semi-permanent colorant solubilizers comprise diethylene glycol monoethyl ether; one or more surface-active agents present in an amount of about 0.25% to about 5% w/w of the composition; and one or more matrix components present in an amount of about 0.5% to about 3.5% w/w of the composition.

In some embodiments, the one or more surface-active agents is PEG 400. In some embodiments, the one or more matrix components is trehalose.

In some embodiments, the inkjet printer ink composition further comprises one or more temporary colorants. In some embodiments, the temporary colorant is present in an amount of about 0.05% to about 0.25% w/w of the composition. In some embodiments, the one or more temporary colorants is FD&C Blue No. 1. Also provided herein is an inkjet printer ink composition comprising: purified genipin present in an amount of about 9% w/w of the composition; one or more volatile solvents present in an amount of about 67% w/w of the composition, wherein the one or more semi-volatile solvents are water and ethanol; one or more semi-volatile semi-permanent colorant solubilizers present in an amount of about 21% w/w of the composition, wherein the one or more semi-volatile semi-permanent colorant solubilizers is diethylene glycol monoethyl ether; one or more surface-active agents present in an amount of about 1% w/w of the composition; and one or more matrix components present in an amount of about 2% w/w of the composition.

In some embodiments, the one or more surface-active agents is PEG 400. In some embodiments, the one or more matrix components is trehalose.

Also provided herein is an inkjet printer ink composition comprising: purified genipin present in an amount of about 9% w/w of the composition; one or more volatile solvents present in an amount of about 66.6% w/w of the composition, wherein the one or more semi-volatile solvents are water and ethanol; one or more semi-volatile semi-permanent colorant solubilizers present in an amount of about 21% w/w of the composition, wherein the one or more semi-volatile semi-permanent colorant solubilizers is diethylene glycol monoethyl ether; one or more surface-active agents present in an amount of about 1% w/w of the composition; one or more matrix components present in an amount of about 2% w/w of the composition; and one or more temporary colorants present in an amount of about 0.2% w/w of the composition.

In some embodiments, the one or more surface-active agents is PEG 400. In some embodiments, the one or more matrix components is trehalose. In some embodiments, the one or more temporary colorants is FD&C Blue No. 1. Also provided herein is an inkjet printer ink composition comprising: purified genipin present in an amount of about 2% to about 5% w/w of the composition; one or more volatile solvents present in an amount of about 65% to about 75% w/w of the composition, wherein the one or more semi-volatile solvents are water and ethanol; one or more semi-volatile semi-permanent colorant solubilizers present in an amount of about 15% to about 25% w/w of the composition, wherein the one or more semi-volatile semi-permanent colorant solubilizers comprise diethylene glycol monoethyl ether; one or more surface-active agents present in an amount of about 0.25% to about 5% w/w of the composition; one or more matrix components present in an amount of about 0.5% to about 3.5% w/w of the composition; and one or more temporary colorants present in an amount of about 0.2% w/w of the composition.

In some embodiments, the one or more surface-active agents is PEG 400. In some embodiments, the one or more matrix components is trehalose. In some embodiments, the one or more temporary colorants is FD&C Blue No. 1.

Also provided herein is an inkjet printer ink composition comprising: purified genipin present in an amount of about 4.5% w/w of the composition; one or more volatile solvents present in an amount of about 70% w/w of the composition, wherein the one or more semi-volatile solvents are water and ethanol; one or more semi-volatile semi-permanent colorant solubilizers present in an amount of about 22.5% w/w of the composition, wherein the one or more semi-volatile semi-permanent colorant solubilizers is diethylene glycol monoethyl ether; one or more surface-active agents present in an amount of about 1% w/w of the composition; one or more matrix components present in an amount of about 2% w/w of the composition; and one or more temporary colorants present in an amount of about 0.1% w/w of the composition.

In some embodiments, the one or more surface-active agents is PEG 400. In some embodiments, the one or more matrix components is trehalose. In some embodiments, the one or more temporary colorants is FD&C Blue No. 1.

Also provided herein is a skin priming composition comprising: one or more volatile solvents; one or more amines; and one or more humectants.

In some embodiments of the skin priming composition, the one or more volatile solvents are selected from the group consisting of: water, methanol, ethanol, isopropanol, and a combination of two or more thereof. In some embodiments of the skin priming composition, wherein the one or more volatile solvents are present in an amount of about 80% to about 98% w/w of the composition. In some embodiments of the skin priming composition, the one or more volatile solvents are present in an amount of about 92% to about 96% w/w of the composition. In some embodiments, the one or more volatile solvents are present in an amount of about 90% to about 95% w/w of the composition. In some embodiments, the one or more volatile solvents are present in an amount of about 93% w/w of the composition.

In some embodiments of the skin priming composition, the one or more amines are selected from the group consisting of: arginine, alanine, asparagine, aspartic acid, cysteine, glutamic acid, glutamine, glycine, histidine, isoleucine, leucine, lysine, methionine, phenylalanine, proline, serine, threonine, tryptophan, tyrosine, valine, δ-aminolevulinic acid, 4-aminobenzoic acid, and γ-aminobutyric acid. In some embodiments of the skin priming composition the one or more amines is L-arginine. In some embodiments of the skin priming composition, the one or more amines are present in an amount of about 0.5% to about 2% w/w of the composition. In some embodiments of the skin priming composition, the one or more amines are present in an amount of about 0.5% to about 1% w/w of the composition. In some embodiments, the one or more amines are present in an amount of about 0.5% to about 2% w/w of the composition. In some embodiments, the one or more amines are present in an amount of about 1% w/w of the composition.

In some embodiments of the skin priming composition the one or more humectants are selected from the group consisting of: glycerin, glycerin, propylene glycol, a polyethylene glycol, a sugar, hexylene glycol, butylene glycol, aloe vera gel, an alpha hydroxy acid such as lactic acid, glyceryl triacetate, lithium chloride, sorbitol, xylitol, maltitol, hyaluronic acid, allantoin, urea, tremella extract, dicyanamide, sodium lactate, and sodium L-pyroglutamate. In some embodiments, the one or more amines is L-arginine.

In some embodiments of the skin priming composition, the one or more humectants is present in an amount of about 1% to about 10% w/w of the composition. In some embodiments of the skin priming composition, the one or more humectants are present in an amount of about 5% w/w of the composition. In some embodiments, the one or more humectants is present in an amount of about 1% to about 10% w/w of the composition. In some embodiments, the one or more humectants are present in an amount of about 5% w/w of the composition.

In some embodiments, the one or more humectants are selected from the group consisting of: glycerin, glycerin, propylene glycol, a polyethylene glycol, a sugar, hexylene glycol, butylene glycol, aloe vera gel, an alpha hydroxy acid such as lactic acid, glyceryl triacetate, lithium chloride, sorbitol, xylitol, maltitol, hyaluronic acid, allantoin, urea, tremella extract, dicyanamide, sodium lactate, and sodium L-pyroglutamate. In some embodiments of the skin priming composition, the one or more humectants is glycerin. In some embodiments, the skin priming composition further comprises one or more preservatives. In some embodiments, the one or more preservatives are present in an amount of about 0.5% to about 1.5% w/w of the composition. In some embodiments, wherein the one or more preservatives are present in an amount of about 1% w/w of the composition. In some embodiments, the one or more preservatives are selected form the group consisting of: ascorbic acid, an ascorbate, a palmitate, citric acid, a benzoate, a benzoic acid, a propionate, propionic acid, a sorbate, sorbic acid, a salicylic acid, a salicylate, hexa-2,4-dienoic acid, a hexa-2,4-dienoate, formaldehyde, a formaldehyde releaser, formic acid and its salts, 3-acetyl-6-methylpyran-2,4-(3H)-dione and its salts, 3,3′-dibromo-4,4′-hexamethylenedioxydibenzamidine and its salts, thiomersal, phenylmercuric salts, undec-10-enoic acid and its salts, 1,3-bis (2-ethylhexyl) hexahydro-5-methyl-5-pyrimidine, 5-bromo-5-nitro-1,3-dioxane, bronopol, 2,4-dichlorobenzyl alcohol, 1-(4-chlorophenyl)-3-(3,4-dichlorophenyl) urea, chlorocresol, chloroxylenol, 5-chloro-2-(2,4-dichlorophenoxy) phenol, N,N″-methylenebis [N′-[3-(hydroxymethyl)-2,5-dioxoimidazolidin-4-yl]urea], polyaminopropyl biguanide, methenamine, quaternium-15, climbazole, DMDM hydantoin, benzyl alcohol, 1-Hydroxy-4-methyl-6-(2,4,4-trimethylpentyl)-2-pyridon, piroctone olamine, bromochlorophene, o-cymen-5-ol, chlorophene, chloroacetaminde, methylchloroisothiazolinone, methylisothiazolinone, phenoxyisopropanol, chlorhexidine, chlorhexidine diacetate, chlorhexidine digluconate, chlorhexidine dihydrochloride, dimethyl oxazolidine, behentrimonium chloride, cetri-monium bromide, cetrimonium chloride, laurtrimonium bromide, laurtrimonium chloride, steartrimonium bromide, steartrimonium chloride, diazolidinyl urea, hexamidine, hexamidine diisethionate, hexamidine paraben, glutaral, 7-ethylbicyclooxazolidine, chlorphenesin, sodium hydroxymethylglycinate, silver chloride, benzethonium chloride, benzalkonium chloride, benza-lkonium bromide, benzalkonium saccharinate, benzylhemiformal, iodopropynyl butylcarbamate, biphenyl-2-ol and its salts, pyrithionine zinc, an erythorbate, a nitrite, ethylenediaminetetraacetic acid (EDTA), butylated hydroxyanisole (BHA), butylated hydroxytoluene (BHT), capryllic acid, dilauryl thiodipropionate, erythorbic acid, gum guaiac, methylparaben, a sulfite, a bisulfate, a metabisulfite, propyl gallatepy, propylparaben, stannous chloride, sulfur dioxide, thiodipropionic acid, an isothiazoline, a paraben, phenoxyethanol, ethylhexylglycerin, a glycols, and a tocopherol. In some embodiments, the one or more preservatives are phenoxyethanol and ethylhexylglycerin.

In some embodiments, the skin priming composition has a pH of about 4 to about 7. In some embodiments, the skin priming composition has a pH of about 4.5 to about 5.5.

In some embodiments of the skin priming composition, the skin priming composition further comprises one or more semi-volatile semi-permanent colorant solubilizers. In some embodiments of the skin priming composition the one or more semi-volatile semi-permanent colorant solubilizers are selected from the group consisting of: a polyethylene glycol, an alkyl glycol, an alkylene glycol ether, and a combination of two or more thereof. In some embodiments of the skin priming composition, the one or more semi-volatile semi-permanent colorant solubilizers is diethylene glycol monoethyl ether. In some embodiments of the skin priming composition, the one or more semi-volatile semi-permanent colorant solubilizers are present in an amount of about 50% to about 90% w/w of the composition.

In some embodiments of the skin priming composition, the skin priming composition further comprises one or more skin penetration enhancers.

In some embodiments of the skin priming composition, the skin priming composition has a pH of about 4 to about 7. In some embodiments of the skin priming composition the skin priming composition has a pH of about 4.5 to about 5.5. In some embodiments of the skin priming composition, the skin priming composition is configured to have a pH of about 4 to about 7. In some embodiments of the skin priming composition the skin priming composition is configured to have a pH of about 4.5 to about 5.5.

Also provided herein is a towelette containing any of the skin priming compositions as described herein.

Also provided herein are adhesive applicator articles comprising an ink composition, e.g., any of the ink compositions described herein; an adhesive; and an adhesive backing. In some embodiments, the ink composition is disposed on and/or coupled to a first surface of the adhesive and the adhesive backing is disposed on and/or coupled to a second surface of the adhesive. In some embodiments, the ink composition is printed by an inkjet printer upon the first surface of the adhesive.

In some embodiments, the adhesive applicator article further comprises a liner. In some embodiments, the liner is comprised of a non-adhesive material.

In some embodiments, the adhesive backing is comprised of a transparent material.

In some embodiments, the adhesive applicator article further comprises an occlusion patch.

In some embodiments, a dimension of the occlusion patch is greater than another dimension corresponding to the adhesive, and wherein the occlusion patch surrounds the adhesive, the composition, or both.

Also provided herein are adhesive applicator articles comprising: an ink composition, e.g., any of the ink compositions described herein; a first adhesive layer; a first backing; a second adhesive layer; and a second backing. In some embodiments, an ink composition, e.g., any of the ink composition described herein, is disposed on and/or coupled to a first surface of the first adhesive layer and the first backing is disposed on and/or coupled to a second surface of the first adhesive layer. In some embodiments, the second adhesive layer is disposed on and/or coupled to a second surface of the first backing.

In some embodiments, a first surface of the second backing is disposed on and/or coupled to a second surface of the second adhesive layer.

In some embodiments, the second adhesive layer and the second backing are disposed on and/or coupled to at least a portion of the second surface of the first backing.

In some embodiments, the ink composition is disposed on and/or coupled to at least a portion of the first surface of the first adhesive layer. In some embodiments, the ink composition is printed by an inkjet printer upon the first surface of the first adhesive.

In some embodiments, the adhesive applicator article further comprises a liner. In some embodiments, the liner is disposed on and/or coupled to the ink composition. In some embodiments, the liner is disposed on and/or coupled to the ink composition and the first surface of the first adhesive, wherein the ink composition is disposed on and/or coupled to a portion of the first surface of the first adhesive layer.

In some embodiments, the adhesive applicator article further comprises an occlusion patch. In some embodiments, a dimension of the occlusion patch is greater than another dimension corresponding to the first adhesive, the second adhesive, or both.

In some embodiments, the liner is comprised of a non-adhesive material.

In some embodiments, the first backing, the second backing, or both are comprised of a transparent material. In some embodiments, the first adhesive layer, the second adhesive layer, or both are comprised of a double-sided adhesive material.

Also provided herein are adhesive applicator articles, comprising: a liner; an adhesive layer to couple to the liner; a stack up to couple to the adhesive layer; a removable liner to contact the stack up; and a border to support the stack up.

In some embodiments, the removable liner exposes the stack up to the environment when removed from the adhesive applicator article. In some embodiments, the removable liner further comprises a pull-tab to facilitate the removal of the liner. In some embodiments, the liner is made of a material comprising fluorosilicone (FS).

In some embodiments, the border is made from a material comprising fluorosilicone (FS). In some embodiments, the border is made from a material comprising fluorosilicone (FS) and is a portion of an occlusion patch.

In some embodiments, a dimension of the occlusion patch is larger than another dimension corresponding to the stack up. In some embodiments, the stack up comprises a different adhesive layer, a double-sided adhesive, and a polyurethane layer.

In some embodiments, the adhesive article further comprises an ink composition, e.g., any of the ink compositions described herein. In some embodiments, the ink composition is printed by an inkjet printer upon the stack up.

Also provided herein are adhesive applicator articles, comprising: an external layer positioned over a plurality of panels; one or more tabs coupled to the plurality of panels; and an end portion of the external layer to receive the one or more tabs.

In some embodiments, each of the plurality of panels comprises: an ink composition, e.g., any of the ink compositions described herein; an adhesive layer; and a backing.

In some embodiments, each of the panels of the plurality of panels are angled a distance from an adjacent different panel of the plurality of panels. In some embodiments, the distance between each of the plurality of panels is the same distance. In some embodiments, the one or more tabs are perpendicular to the plurality of panels.

Also provided herein are adhesive applicator articles comprising: an external layer positioned over an ink composition, e.g., any of the ink compositions described herein; a first portion of the external layer and a second portion of the external layer; and a first side of the first portion and a second side of the first portion.

In some embodiments, each of the plurality of panels comprises: an adhesive layer; and a backing.

In some embodiments, the ink composition is printed by an inkjet printer upon the adhesive layer.

In some embodiments, the first side of the first portion and the second side of the first portion are flexible and couple together. In some embodiments, the second portion is perpendicular to the first portion. In some embodiments, the second portion include one or more curved portions.

In some embodiments, wherein the one or more curved portions are curved at different radius of curvature.

In some embodiments, the ink composition forms a pattern on the first portion. In some embodiments, the ink composition forms a pattern on the second portion. In some embodiments, the ink composition forms a pattern on the first portion and the second portion.

In some embodiments, the external layer is made from a transparent material.

Also provided herein is a kit comprising: any of the adhesive applicator articles as described herein; and any of the skin priming compositions as described herein.

Also provided herein is a kit comprising: any of the adhesive applicator articles as described herein; and a towelette containing any of the skin priming compositions as described herein.

The details of one or more embodiments of the invention are set forth in the accompanying drawings and the description below. Other features, objects, and advantages of the invention will be apparent from the description and drawings, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 is a schematic side cross-sectional view of an exemplary article according to one embodiment of the present disclosure.

FIG. 2A is an exploded perspective view of an exemplary article according to another embodiment of the present disclosure.

FIG. 2B is a side cross-sectional view of the exemplary article of FIG. 2A.

FIG. 3 is an exploded perspective view of an adhesive applicator article according to another embodiment of the present disclosure.

FIG. 4 is a view of an adhesive applicator article according to another embodiment of the present disclosure.

FIG. 5A shows a first scale, Scale 1, of genipin color development indicators without blue dye present: A. yellow; B. amber; C. deep green; D. deep teal; E. deep blue.

FIG. 5B shows a second scale, Scale 2, of genipin color development indicators with blue dye present: A. no color; B. sky blue; C. light blue; D. royal blue; E. navy blue.

FIG. 6 is a schematic showing the test sites for article application on the forearm or upper arm.

FIG. 7 is a schematic of a photography suite.

FIG. 8A shows an ink intensity scale.

FIG. 8B shows an ink density scale.

FIG. 9 is a plot showing the amount of genipin extracted from each dried printed adhesive substrate.

FIG. 10 shows the enhanced color density of genipin ink as a result of a skin priming treatment.

FIG. 11 shows the color density of genipin ink as a result of a skin priming treatment or genipin transfer using a heat pad.

FIG. 12 is an exploded perspective view of an adhesive applicator article according to another embodiment of the present disclosure.

FIG. 13 is another exploded perspective view of an adhesive applicator article according to another embodiment of the present disclosure.

FIGS. 14A, 14B, and 14C are perspective drawings of an adhesive applicator article according to an embodiment of the present disclosure.

FIGS. 15A, 15B, and 15C are perspective drawings of an adhesive applicator article according to an embodiment of the present disclosure.

FIGS. 16A, 16B, and 16C are perspective drawings of an adhesive applicator article according to an embodiment of the present disclosure.

FIGS. 17A, 17B, and 17C are perspective drawings of an adhesive applicator article according to an embodiment of the present disclosure.

FIGS. 18A, 18B, and 18C are perspective drawings of an adhesive applicator article according to an embodiment of the present disclosure.

FIG. 19 is another example of an adhesive applicator article according to another embodiment of the present disclosure.

FIGS. 20A and 20B illustrate perspective views of an example adhesive applicator article according to another embodiment of the present disclosure.

FIG. 21 is another example of an adhesive applicator article according to another embodiment of the present disclosure.

FIGS. 22A and 22B illustrate another example of an adhesive applicator article according to another embodiment of the present disclosure.

FIGS. 23A and 23B illustrate another example of an adhesive applicator article according to another embodiment of the present disclosure.

FIG. 24 shows the general trend for the longevity of a semi-permanent tattoo on various body areas.

FIGS. 25A and 25B shows that semi-permanent tattoo performance can be skin tone dependent.

### DETAILED DESCRIPTION

The manufacturing approach employing ink-jet printing directly onto a skin transfer substrate enables the rapid production of made-to-order custom designed semi-permanent tattoos. However, inkjet printing can impose constraints on the physico-chemical properties of the ink dispensed by the printer nozzles. In addition, the ink vehicle should be formulated such that the printed ink on the transfer substrate is safe for skin contact and sustains stability or skin transferability for the duration of the product shelf life. The semi-permanent tattoo longevity, or the semi-permanent colorant residence time in the skin, may be limited by the natural skin desquamation process, and the thickness of each layer of the skin (the thickness of the epidermis can depend on the body region).

In some embodiments, the ink compositions, e.g., genipin-based ink compositions, as well as layered adhesive articles and methods for application of the semi-permanent tattoos described herein can provide a single-step semi-permanent tattoo application process. This can encourage user adoption and improved semi-permanent tattoo transfer and development by improving ease of generating a successful semi-permanent tattoo result. Moreover, the use of ink jet printing technology to manufacture the product can enable the swift and economical production of made to order, custom semi-permanent tattoo products with prolonged shelf life.

Reference to the term “about” has its usual meaning in the context of compositions to allow for reasonable variations in amounts that can achieve the same effect and also refers herein to a value of plus or minus 10% of the provided value. For example, “about 20” means or includes amounts from 18 to and including 22.

As used herein, a “colorant” refers to a substance that changes the color of reflected or transmitted light as the result of wavelength-selective absorption.

As used herein, a “semi-permanent colorant” refers to a colorant that penetrates one or more layers of the skin and is unable to be removed from the skin without physical disruption or natural desquamation of the skin. For example, a semi-permanent colorant may penetrate the stratum corneum and react with other molecules, e.g., other colorant molecules and/or other molecules present in the stratum corneum, such that the semi-permanent colorant is immobilized within the stratum corneum. For example, the semi-permanent colorant may cross-link with collagen found in the stratum corneum. In some embodiments, the semi-permanent colorant may react with other molecules, e.g., other colorant molecules and/or other molecules present in the stratum corneum, to form a molecule that is too large to diffuse out of the stratum corneum. In some embodiments, wherein the semi-permanent colorant penetrates the stratum corneum, the colorant residence time is determined by the natural skin desquamation process. In some embodiments, the semi-permanent colorant cannot be washed off, for example, by water, soap, and/or isopropanol. In some embodiments, a semi-permanent colorant includes a natural or synthetic pigment or dye. In some embodiments, a semi-permanent colorant can include a colorant precursor, for example, a molecule, such as genipin, that expresses color upon reaction with one or more other molecules.

As used herein, a “temporary colorant” refers to a colorant that sits on top of the skin or, if it penetrates the skin, can diffuse out of the skin or can be washed off by, for example, water, soap, and/or isopropanol.

As used herein, “color” refers to wavelengths of electromagnetic radiation visible to the human eye.

“Surface-active agent” or “surfactant” as used herein refers to a substance that tends to reduce the surface tension of a liquid in which it is dissolved.

A “surface” as described herein refers to a portion of a layer of material (e.g., backing, adhesive backing, an adhesive layer, etc.) that may be substantially parallel to a different layer. In some examples, the surface area of a surface of a particular layer described herein may be larger than the surface of an edge of the particular layer. For example, an edge of a particular layer can be substantially perpendicular to a surface of the particular layer. As used herein, the term “substantially” intends that the characteristic needs not be absolute, but is close enough so as to achieve the advantages of the characteristic. For example, “substantially parallel” is not limited to absolute parallelism, and can include orientations that are intended to be parallel but due to manufacturing limitations may not be precisely parallel. For example, “substantially parallel” features are at least closer to a parallel orientation than a perpendicular orientation, and generally are formed within a few degrees of parallel. Similarly, “substantially perpendicular” is not limited to absolute perpendicularity, and can include orientations that are intended to be parallel but due to manufacturing limitations may not be precisely perpendicular. For example, “substantially perpendicular” features are at least closer to a perpendicular orientation than a parallel orientation, e.g., within a few degrees of perpendicular.

Provided herein are ink compositions comprising one or more semi-permanent colorants; one or more volatile solvents; and one or more semi-volatile semi-permanent colorant solubilizers.

In some embodiments, the one or more semi-permanent colorants are present in an amount of about 0.1% to about 20% w/w of the composition. For example, about 0.1% to about 18%, about 0.1% to about 16%, about 0.1% to about 14%, about 0.1% to about 12%, about 0.1% to about 10%, about 0.1% to about 8%, about 0.1% to about 6%, about 0.1% to about 4%, about 0.1% to about 2%, about 16% to about 18%, about 14% to about 18%, about 12% to about 18%, about 10% to about 18%, about 8% to about 18%, about 6% to about 18%, about 4% to about 18%, or about 2% to about 18% w/w of the composition. In some embodiments, the one or more semi-permanent colorants are present in an amount of about 5% to about 15%, about 5% to about 10%, about 7% to about 11%, about 5% to about 7%, about 6% to about 8%, about 7% to about 9%, about 8% to about 10%, or about 6.5% to about 7.5% w/w of the composition. In some embodiments, the one or more semi-permanent colorants are present in an amount of about 1%, about 2%, about 3%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 15%, about 16%, about 17%, about 18%, about 19%, or about 20% w/w of the composition.

In some embodiments, the one or more semi-permanent colorants comprise a colorant precursor. In some embodiments, the colorant precursor is genipin. Accordingly, in some embodiments, the semi-permanent colorant is genipin. As used herein, “genipin” refers to genipin extract, partially purified genipin extract, and/or purified genipin, as described further herein. Genipin is derived from an iridoid glycoside called geniposide, which is present in nearly 40 plant species. Additionally, genipin is a precursor to gardenia blue that undergoes a free radical polymerization and/or oligomerization reaction upon exposure to amine groups to form a blue colorant (see, for example, Touyama et al., Chem. Pharm. Bull. 42:668-673, 1994; and Touyama et al., Chem. Pharm. Bull. 42(8):1571-1578, 1994; both of which are incorporated by reference herein in their entireties). When placed in contact with skin, genipin can react with the amines in skin to generate the polymer-based and/or oligomer-based blue color in situ. The penetration profile of genipin can dictate the color concentration profile in the stratum corneum as the polymer-based and/or oligomer-based blue colorant is too large to penetrate the skin at relevant concentrations. For example, the average molecular weight of the polymer-based and/or oligomer-based blue colorant has been reported to be 8900±600 Daltons, and the polymer-based and/or oligomer-based blue colorant comprises 40 to 44 monomer units (see, for example, Touyama et al., Chem. Pharm. Bull. 42:668-673, 1994). In some embodiments, the average molecular weight of the polymerized and/or oligomerized genipin present in the compositions described herein can range from about 200 Daltons to about 15000 Daltons. In some embodiments, the average molecular weight of the polymerized and/or oligomerized genipin present in the compositions described herein can range from about 500 Daltons to about 15000 Daltons. In some embodiments, a plurality of different molecular weights of genipin can be used. For example, in some embodiments, the compositions described herein can comprise genipin having an average molecular weight from about 200 Daltons to about 15000 Daltons. In some embodiments, the compositions described herein can comprise genipin having an average molecular weight of from about 200 to about 1000 Daltons, about 500 to about 1500 Daltons, about 1000 to about 2000 Daltons, about 1500 to about 2500 Daltons, about 2000 to about 3500 Daltons, 3500 to about 5000 Daltons, 5000 to about 6500 Daltons, 6000 to about 8300 Daltons, from about 8300 to about 9500 Daltons, and combinations thereof.

In some embodiments of the ink compositions described herein, the genipin is present in an amount of about 0.1% to about 20% w/w of the composition. For example, about 0.1% to about 18%, about 0.1% to about 16%, about 0.1% to about 14%, about 0.1% to about 12%, about 0.1% to about 10%, about 0.1% to about 8%, about 0.1% to about 6%, about 0.1% to about 4%, about 0.1% to about 2%, about 16% to about 18%, about 14% to about 18%, about 12% to about 18%, about 10% to about 18%, about 8% to about 18%, about 6% to about 18%, about 4% to about 18%, or about 2% to about 18% w/w of the composition. In some embodiments, the genipin is present in an amount of about 5% to about 15%, about 5% to about 10%, about 7% to about 11%, about 5% to about 7%, about 6% to about 8%, about 7% to about 9%, about 8% to about 10%, or about 6.5% to about 7.5% w/w of the composition. In some embodiments, the genipin is present in an amount of about 1%, about 2%, about 3%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 15%, about 16%, about 17%, about 18%, about 19%, or about 20% w/w of the composition.

In some embodiments, the genipin is purified genipin. The presence of amines, for example, amines that are endogenously present in plant extracts or exogenously introduced, in a genipin solution may give rise to shelf life stability challenges. For example, with time, the reactive precursors can develop into the polymer-based and/or oligomer-based blue semi-permanent colorant prior to application to the skin, limiting the penetration of the semi-permanent colorant to the stratum corneum. Penetration of the genipin into the deeper layers of the stratum corneum can allow for the subsequent development and retention of the polymer-based and/or oligomer-based blue semi-permanent colorant in the skin, which may lead to increased semi-permanent tattoo longevity. The semi-permanent tattoo longevity, or the semi-permanent colorant residence time in the skin, may then be limited by the natural skin desquamation process. In some embodiments, the purified genipin has a purity of greater than about 85% w/w. For example, the purified genipin has a purity of greater than about 86%, about 88%, about 90%, about 92%, about 94%, about 96%, about 98%, about 99%, or about 99.5% w/w.

In some embodiments of the ink composition described herein, the purified genipin is present in an amount of about 0.1% to about 20% w/w of the composition. For example, about 0.1% to about 18%, about 0.1% to about 16%, about 0.1% to about 14%, about 0.1% to about 12%, about 0.1% to about 10%, about 0.1% to about 8%, about 0.1% to about 6%, about 0.1% to about 4%, about 0.1% to about 2%, about 16% to about 18%, about 14% to about 18%, about 12% to about 18%, about 10% to about 18%, about 8% to about 18%, about 6% to about 18%, about 4% to about 18%, about 2% to about 18%, 5% to about 15%, or about 5% to about 10% w/w of the composition. In some embodiments, the purified genipin is present in an amount of about 5% to about 15%, about 5% to about 10%, about 7% to about 11%, about 5% to about 7%, about 6% to about 8%, about 7% to about 9%, about 8% to about 10%, or about 6.5% to about 7.5% w/w of the composition. In some embodiments of the ink composition described herein, the purified genipin is present in an amount of about 1%, about 2%, about 3%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 15%, about 16%, about 17%, about 18%, about 19%, or about 20% w/w of the composition.

Volatile solvents can include liquids that vaporize at room temperature. Non-limiting examples of volatile solvents include water, methanol, ethanol, and isopropanol. In some embodiments, the solvent may be selected to have a low toxicity profile when used as described herein. For example, the solvent may be selected such that any residual solvent left after the ink has dried on an applicator is not in a toxic amount for contact with a human skin surface.

In some embodiments of the ink composition described herein, the one or more volatile solvents are present in an amount of about 0.1% to about 95% w/w of the composition. For example, about 10% to about 95%, about 20% to about 95%, about 30% to about 95%, about 40% to about 95%, about 50% to about 95%, about 60% to about 95%, about 70% to about 95%, about 80% to about 95%, about 10% to about 20%, about 10% to about 30%, about 10% to about 40%, about 10% to about 50%, about 10% to about 60%, about 10% to about 70%, about 10% to about 80%, or about 10% to about 90% w/w of the composition. In some embodiments of the ink composition described herein, the one or more volatile solvents are present in an amount of about 55% to about 75%, about 60% to about 80%, about 65% to about 85%, about 70% to about 90%, about 75% to about 95%, about 50% to about 75%, about 55% to about 80%, about 60% to about 85%, about 65% to about 90%, about 60% to about 70%, about 65% to about 75%, about 70% to about 80%, or about 75% to about 85% w/w of the composition. For example, about 60% to about 65%, about 62% to about 67%, about 65% to about 70%, about 67% to about 72%, about 70% to about 75%, about 72% to about 77%, about 75% to about 80%, about 77% to about 82%, about 80% to about 85%, about 82% to about 87%, about 85% to about 90%, about 87% to about 92%, or about 90% to about 95% w/w of the composition. In some embodiments of the ink composition described herein, the one or more volatile solvents are present in an amount of about 50%, about 55%, about 60%, about 65%, about 66%, about 67%, about 68%, about 69%, about 70%, about 71%, about 72%, about 73%, about 74%, about 75%, about 76%, about 77%, about 78%, about 79%, about 80%, about 85%, about 90%, or about 95% w/w of the composition.

In some embodiments, the one or more volatile solvents can be a solvent in which the semi-permanent colorant, e.g., a colorant precursor such as genipin, is soluble. For example in some embodiments of the ink compositions described herein, the one or more volatile solvents is a solvent in which the semi-permanent colorant, e.g., a colorant precursor such as genipin has a solubility of at least 5% w/w of the composition, for example, at least 6%, at least 7%, at least 8%, or at least 9% w/w of the composition. In some embodiments of the ink compositions described herein, the semi-permanent colorant, e.g., a colorant precursor such as genipin has a solubility of about 9% w/w of the composition. In some embodiments, the one or more volatile solvents are selected from the group consisting of: water, methanol, ethanol, and isopropanol.

In some embodiments of the ink compositions described herein, a combination of volatile solvents can be used. In some embodiments, each volatile solvent can be independently present in an amount of about 5% to about 80%. For example, about 5% to about 10%, about 5% to about 20%, about 5% to about 30%, about 5% to about 40%, about 5% to about 50%, about 5% to about 60%, about 5% to about 70%, about 70% to about 80%, about 60% to about 80%, about 50% to about 80%, about 40% to about 80%, about 30% to about 80%, about 20% to about 80%, or about 10% to about 80% w/w of the composition. In some embodiments, a volatile solvent can be independently present in an amount of about 20% to about 30%, about 25% to about 35%, about 30% to about 40%, about 35% to about 45%, about 40% to about 50%, or about 45% to about 50% w/w of the composition. For example, about 20%, about 25%, about 26%, about 27%, about 28%, about 29%, about 30%, about 31%, about 32%, about 33%, about 34%, about 35%, about 36%, about 3′7%, about 38%, about 39%, about 40%, about 41%, about 42%, about 43%, about 44%, about 45%, about 46%, about 47%, about 48%, about 49%, or about 50% w/w of the composition.

In some embodiments of the ink compositions described herein, the one or more volatile solvents can include a combination of water and ethanol. In some embodiments, the water can be independently present in an amount of about 40% to about 55% w/w of the composition and the ethanol can be independently present in an amount of about 15% to about 30% w/w of the composition. In some embodiments, the water independently can be present in an amount of about 45% to about 50% w/w of the composition and the independently ethanol can be present in an amount of about 20% to about 27% w/w of the composition. In some embodiments, the water can be independently present in an amount of about 25% to about 40% w/w of the composition and the ethanol can be independently present in an amount of about 35% to about 50% w/w of the composition. In some embodiments, the water can be independently present in an amount of about 25% to about 35% w/w of the composition and the ethanol can be independently present in an amount of about 35% to about 45% w/w of the composition. In some embodiments, the water can be independently present in an amount of about 28% to about 32% w/w of the composition and the ethanol can be independently present in an amount of about 40% to about 45% w/w of the composition.

A “semi-volatile semi-permanent colorant solubilizer” as used herein refers to a semi-volatile solvent in which the semi-permanent colorant, e.g., a colorant precursor such as genipin, is soluble. In some embodiments of the compositions described herein, the semi-permanent colorant, e.g., a colorant precursor such as genipin exhibits a solubility of greater than 5 mg/mL in the one or more semi-volatile semi-permanent colorant solubilizers. In some embodiments of the compositions described herein, genipin exhibits a solubility of greater than 5 mg/mL in the one or more semi-volatile semi-permanent colorant solubilizers. Delivery of semi-permanent colorant into the upper skin layers through passive diffusion is driven by the solubility of the semi-permanent colorant in the delivery vehicle and the skin permeability. As such, solvents that enhance the semi-permanent colorant solubility in water can help increase the penetration of the semi-permanent colorant into the stratum corneum. Non-limiting examples of a semi-volatile semi-permanent colorant solubilizer include a polyethylene glycol, an alkyl glycol, and an alkylene glycol ether. An alkylene glycol ether can include ethylene glycol dimethyl ether, ethylene glycol diethyl ether, and ethylene glycol dibutyl ether.

In some embodiments of the ink compositions described herein, the one or more semi-volatile semi-permanent colorant solubilizers are present in an amount of about 0.1% to about 95% w/w of the composition. For example, about 1% to about 95%, about 5% to about 95%, about 10% to about 95%, about 20% to about 95%, about 30% to about 95%, about 40% to about 95%, about 50% to about 95%, about 60% to about 95%, about 70% to about 95%, about 80% to about 95%, about 10% to about 20%, about 10% to about 30%, about 10% to about 40%, about 10% to about 50%, about 10% to about 60%, about 10% to about 70%, about 10% to about 80%, or about 10% to about 90% w/w of the composition. In some embodiments of the ink compositions described herein, the one or more semi-volatile semi-permanent colorant solubilizers are present in an amount of about 1% to about 30%, about 5% to about 35%, about 10% to about 40%, about 1% to about 20%, about 5% to about 20%, about 10% to about 25%, about 15% to about 30%, about 30% to about 35%, about 25% to about 40%, about 1% to about 10%, about 5% to about 15%, about 10% to about 20%, or about 15% to about 25% w/w of the composition. For example, about 10% to about 15%, about 12% to about 17%, about 15% to about 20%, about 17% to about 22%, about 20% to about 25%, about 22% to about 27%, about 25% to about 30%, about 27% to about 32%, about 30% to about 35%, about 32% to about 37%, or about 35% to about 40% w/w of the composition. In some embodiments of the ink compositions described herein, the one or more semi-volatile semi-permanent colorant solubilizers are present in an amount of about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% w/w of the composition.

In some embodiments of the ink compositions described herein, the one or more semi-volatile semi-permanent colorant solubilizers are selected from the group consisting of: a polyethylene glycol, an alkyl glycol, an alkylene glycol ether, and a combination of two or more thereof.

In some embodiments of the ink compositions described herein, the one or more semi-volatile semi-permanent colorant solubilizers comprise an alkyl glycol. In some embodiments, the alkyl glycol is diethylene glycol monoethyl ether (e.g., ethoxydiglycol, TRANSCUTOL® CG). In some embodiments, diethylene glycol monoethyl ether is present in an amount of about 1% to about 30%, about 5% to about 35%, about 10% to about 40%, about 1% to about 20%, about 5% to about 20%, about 10% to about 25%, about 15% to about 30%, about 30% to about 35%, about 25% to about 40%, about 1% to about 10%, about 5% to about 15%, about 10% to about 20%, or about 15% to about 25% w/w of the composition. For example, about 10% to about 15%, about 12% to about 17%, about 15% to about 20%, about 17% to about 22%, about 20% to about 25%, about 22% to about 27%, about 25% to about 30%, about 27% to about 32%, about 30% to about 35%, about 32% to about 37%, or about 35% to about 40% w/w of the composition. In some embodiments, diethylene glycol monoethyl ether is present in an amount of about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% w/w of the composition.

In some embodiments of the ink compositions described herein, the one or more semi-volatile semi-permanent colorant solubilizers is diethylene glycol monoethyl ether.

In some embodiments of the ink compositions described herein, the composition has a viscosity from about 1 to about 20 centipoise. For example, about 1 to about 19, about 1 to about 18, about 1 to about 17, about 1 to about 16, about 1 to about 15, about 1 to about 14, about 1 to about 13, about 1 to about 12, about 1 to about 11, about 1 to about 10, about 1 to about 9, about 1 to about 8, about 1 to about 7, about 1 to about 6, about 1 to about 5, about 1 to about 4, about 1 to about 3, about 1 to about 2, about 19 to about 20, about 18 to about 20, about 17 to about 20, about 16 to about 20, about 15 to about 20, about 14 to about 20, about 13 to about 20, about 12 to about 20, about 11 to about 20, about 10 to about 20, about 9 to about 20, about 8 to about 20, about 7 to about 20, about 6 to about 20, about 5 to about 20, about 4 to about 20, about 3 to about 20 centipoise, or about 2 to about 20 centipoise. In some embodiments, the composition has a viscosity from about 5 to about 15, about 5 to about 10, about 10 to about 15, about 2 to about 4, about 3 to about 5, or about 4 to about 6 centipoise. In some embodiments of the ink compositions described herein, the composition has a viscosity of about 3, about 3.25, about 3.5, about 3.75, about 4, about 4.25, about 4.5, about 4.75, about 5, about 5.25, about 5.5, about 5.75, about 6, about 6.25, about 6.5, about 6.75, about 7, about 8, about 9, about 10, about 11, about 12, about 13, about 14, about 15, about 16, about 17, about 18, about 19, or about 20 centipoise.

In some embodiments of the ink compositions described herein, the composition is configured to have a viscosity from about 1 to about 7 centipoise. For example, about 1 to about 6, about 1 to about 5, about 1 to about 4, about 1 to about 3, about 1 to about 2, about 6 to about 7, about 5 to about 7, about 4 to about 7, about 3 to about 7 centipoise, or about 2 to about 7 centipoise. In some embodiments, the composition is configured to have a viscosity from about 2 to about 4, about 3 to about 5, or about 4 to about 6 centipoise. In some embodiments of the ink compositions described herein, the composition is configured to have a viscosity of about 3, about 3.25, about 3.5, about 3.75, about 4, about 4.25, about 4.5, about 4.75, about 5, about 5.25, about 5.5, about 5.75, about 6, about 6.25, about 6.5, about 6.75, about 7, about 8, about 9, about 10, about 11, about 12, about 13, about 14, about 15, about 16, about 17, about 18, about 19, or about 20 centipoise.

In some embodiments of the ink compositions described herein, the composition has a surface tension from about 10 to about 45 dynes/cm. For example, about 10 to about 20, about 10 to about 30, about 10 to about 45, about 40 to about 45, about 30 to about 45, or about 20 to about 45 dynes/cm. In some embodiments of the ink compositions described herein, the composition has a surface tension from about 15 to about 25, about 20 to about 30, about 25 to about 35, about 30 to about 40, about 35 to about 45, or about 20 to about 35 dynes/cm. For example, about 20, about 21, about 22, about 23, about 24, about 25, about 26, about 27, about 28, about 29, about 30, about 31, about 32, about 33, about 34, or about 35 dynes/cm.

In some embodiments of the ink compositions described herein, the composition is configured to have a surface tension from about 10 to about 45 dynes/cm. For example, about 10 to about 20, about 10 to about 30, about 10 to about 45, about 40 to about 45, about 30 to about 45, or about 20 to about 45 dynes/cm. In some embodiments of the ink compositions described herein, the composition is configured to have a surface tension from about 15 to about 25, about 20 to about 30, about 25 to about 35, about 30 to about 40, about 35 to about 45, or about 20 to about 35 dynes/cm. For example, about 20, about 21, about 22, about 23, about 24, about 25, about 26, about 27, about 28, about 29, about 30, about 31, about 32, about 33, about 34, or about 35 dynes/cm.

In some embodiments of the ink compositions described herein, the composition further comprises one or more surface-active agents. Non-limiting examples of a surface-active agent include an alkylbenzene sulfonate, an alkyl sulfate, an alkyl ether sulfate, a soap, an ethoxylate, an alkyl alcohol, a lignosulfonate, and a triglyceride. For example, a surface-active agent can include sodium dodecyl benzenesulfonate, lauryl sulfate, di-alkyl sulfosuccinate, dimethyl ether of tetradecyl phosphonic, lauryl mono-ethanol, glycerol diester (diglyceride), dodecyl betaine, abietic acid, polyethoxylated octyl phenol, 1,2-hexanediole (e.g., OPTIPHEN® HD), and sorbitan monoester.

In some embodiments of the ink compositions described herein, the composition further comprises one or more surface-active agents. In some embodiments, the one or more surface-active agents are present in an amount of about 0.1% to about 10% w/w of the composition. For example, about 0.1% to about 8%, about 0.1% to about 6%, about 0.1% to about 5%, about 0.1% to about 4%, about 0.1% to about 3%, about 0.1% to about 2%, about 0.1% to about 1%, about 8% to about 10%, about 6% to about 10%, about 4% to about 10%, about 3% to about 10%, about 2% to about 10%, or about 1% to about 10% w/w of the composition. In some embodiments of the ink compositions described herein, the one or more surface-active agents are present in an amount of about 0.25% to about 5% w/w of the composition. For example, about 0.25% to about 5%, about 0.25% to about 4%, about 0.25% to about 3%, about 0.25% to about 2%, about 0.25% to about 1%, about 2% to about 5%, about 2% to about 4%, about 2% to about 3%, about 1% to about 4%, about 1% to about 3%, about 1% to about 2%, about 3% to about 5%, about 4% to about 5%, about 3% to about 5%, about 2% to about 5%, or about 1% to about 5% w/w of the composition. In some embodiments, the one or more surface-active agents are present in an amount of about 0.25%, about 0.5%, about 1%, about 1.5%, about 2%, about 2.5%, about 3%, about 3.5%, about 4%, about 4.5%, about 5%, about 5.5%, about 6%, about 7%, about 8%, about 9%, or about 10% w/w of the composition.

In some embodiments of the ink compositions described herein, the one or more surface-active agents are selected from the group consisting of: an alkylbenzene sulfonate, a polyethylene glycol, an alkyl sulfate, an alkyl ether sulfate, a soap, an ethoxylate, a lignosulfonate, and a triglyceride. In some embodiments, the one or more surface-active agents are selected from the group consisting of: sodium dodecyl benzenesulfonate, lauryl sulfate, di-alkyl sulfosuccinate, dimethyl ether of tetradecyl phosphonic, lauryl mono-ethanol, glycerol diester (diglyceride), dodecyl betaine, abietic acid, polyethoxylated octyl phenol, 1,2-hexanediol, and sorbitan monoester.

In some embodiments of the ink compositions described herein, the one or more surface-active agents is 1,2-hexanediol. In some embodiments, 1,2-hexanediol is present in an amount of about 0.1% to about 10% w/w of the composition. For example, about 0.1% to about 8%, about 0.1% to about 6%, about 0.1% to about 5%, about 0.1% to about 4%, about 0.1% to about 3%, about 0.1% to about 2%, about 0.1% to about 1%, about 8% to about 10%, about 6% to about 10%, about 4% to about 10%, about 3% to about 10%, about 2% to about 10%, or about 1% to about 10% w/w of the composition. In some embodiments of the ink compositions described herein, 1,2-hexanediol is present in an amount of about 0.25% to about 5% w/w of the composition. For example, about 0.25% to about 5%, about 0.25% to about 4%, about 0.25% to about 3%, about 0.25% to about 2%, about 0.25% to about 1%, about 2% to about 5%, about 2% to about 4%, about 2% to about 3%, about 1% to about 4%, about 1% to about 3%, about 1% to about 2%, about 3% to about 5%, about 4% to about 5%, about 3% to about 5%, about 2% to about 5%, or about 1% to about 5% w/w of the composition. In some embodiments, 1,2-hexanediol is present in an amount of about 0.25%, about 0.5%, about 1%, about 1.5%, about 2%, about 2.5%, about 3%, about 3.5%, about 4%, about 4.5%, about 5%, about 5.5%, about 6%, about 7%, about 8%, about 9%, or about 10% w/w of the composition.

In some embodiments of the ink compositions described herein, the one or more surface-active agents is polyethylene glycol 400 (PEG 400). In some embodiments, the PEG 400 is present in an amount of about 0.1% to about 10% w/w of the composition. For example, about 0.1% to about 8%, about 0.1% to about 6%, about 0.1% to about 5%, about 0.1% to about 4%, about 0.1% to about 3%, about 0.1% to about 2%, about 0.1% to about 1%, about 8% to about 10%, about 6% to about 10%, about 4% to about 10%, about 3% to about 10%, about 2% to about 10%, or about 1% to about 10% w/w of the composition. In some embodiments of the ink compositions described herein, the PEG 400 is present in an amount of about 0.25% to about 5% w/w of the composition. For example, about 0.25% to about 5%, about 0.25% to about 4%, about 0.25% to about 3%, about 0.25% to about 2%, about 0.25% to about 1%, about 2% to about 5%, about 2% to about 4%, about 2% to about 3%, about 1% to about 4%, about 1% to about 3%, about 1% to about 2%, about 3% to about 5%, about 4% to about 5%, about 3% to about 5%, about 2% to about 5%, or about 1% to about 5% w/w of the composition. In some embodiments of the ink compositions described herein, the PEG 400 is present in an amount of about 0.25%, about 0.5%, about 1%, about 1.5%, about 2%, about 2.5%, about 3%, about 3.5%, about 4%, about 4.5%, about 5%, about 5.5%, about 6%, about 7%, about 8%, about 9%, or about 10% w/w of the composition.

In some embodiments of the ink compositions described herein, the composition further comprises one or more matrix components. A matrix component can form a solid or semi-solid, immobilized matrix on a substrate. For example, after an ink composition as described herein is applied to a substrate and the ink dries, e.g., the volatile solvents have evaporated or nearly evaporated off of the substrate, the matrix component can form a solid or semi-solid, immobilized matrix on a substrate. Non-limiting examples of a matrix component include a sugar, a sugar alcohol (e.g., sorbitol, mannitol, xylitol, isomalt, hydrogenated starch hydrolysates), a polymer, or a combination of two or more thereof. For example, a sugar can include a monosaccharide (e.g., glucose, fructose, galactose, ribose, and xylose), a disaccharide (e.g., trehalose such as TREHALOSE 100, sucrose, lactotrehalose, galactotrehalose, 6-azidotrehalose, maltose, lactose, lactulose, cellobiose, and chitobiose), or an oligosaccharide (e.g., starch, glycogen, cellulose, and chitin). In some embodiments, the polymer can include pullulan, a cellulose derivative (e.g., hydroxypropylmethylcellulose, hydroxyethylcellulose, and hydroxymethylcellulose), an acrylate, polyvinylalcohol, an ethylene oxide/propylene oxide copolymer, and polyester.

In some embodiments of the ink compositions described herein, the one or more matrix components are present in an amount of about 0.1% to about 50% w/w of the composition. For example, about 0.1% to about 25%, about 25% to about 50%, or about 15% to about 35% w/w of the composition. In some embodiments of the ink compositions described herein, the one or more matrix components are present in an amount of about 0.1% to about 10% w/w of the composition. For example, about 0.1% to about 8%, about 0.1% to about 6%, about 0.1% to about 4%, about 0.1% to about 3%, about 0.1% to about 2%, about 0.1% to about 1%, about 8% to about 10%, about 6% to about 10%, about 4% to about 10%, about 2% to about 10%, about 1% to about 5%, about 1% to about 4%, about 1% to about 3%, about 1% to about 2%, about 2% to about 5%, about 2% to about 4%, about 2% to about 3%, or about 3% to about 5% w/w of the composition. In some embodiments of the ink compositions described herein, the one or more matrix components are present in an amount of about 0.1%, about 0.5%, about 1%, about 1.5%, about 2%, about 2.5%, about 3%, about 3.5%, about 4%, about 4%, about 4.5%, about 5%, about 5.5%, about 6%, about 7%, about 8%, about 9%, or about 10% w/w of the composition.

In some embodiments of the ink compositions described herein, the one or more matrix components are selected from the group consisting of: a sugar, a sugar alcohol, a polymer, or a combination of two or more thereof. In some embodiments, the one or more matrix components are selected from the group consisting of: sorbitol, mannitol, xylitol, isomalt, hydrogenated starch hydrolysates, glucose, fructose, galactose, ribose, xylose, trehalose, sucrose, lactotrehalose, galactotrehalose, 6-azidotrehalose, maltose, lactose, lactulose, cellobiose, chitobiose, starch, glycogen, cellulose, chitin, pullulan, hydroxypropylmethylcellulose, hydroxyethylcellulose, hydroxymethylcellulose, an acrylate, polyvinylalcohol, an ethylene oxide/propylene oxide copolymer, and polyester.

In some embodiments of the ink compositions described herein, the one or more matrix components is trehalose. In some embodiments, trehalose is present in an amount of about 0.1% to about 8%, about 0.1% to about 6%, about 0.1% to about 4%, about 0.1% to about 3%, about 0.1% to about 2%, about 0.1% to about 1%, about 8% to about 10%, about 6% to about 10%, about 4% to about 10%, about 2% to about 10%, about 1% to about 5%, about 1% to about 4%, about 1% to about 3%, about 1% to about 2%, about 2% to about 5%, about 2% to about 4%, about 2% to about 3%, or about 3% to about 5% w/w of the composition. In some embodiments, trehalose is present in an amount of about 0.1%, about 0.5%, about 1%, about 1.5%, about 2%, about 2.5%, about 3%, about 3.5%, about 4%, about 4%, about 4.5%, about 5%, about 5.5%, about 6%, about 7%, about 8%, about 9%, or about 10% w/w of the composition.

In some embodiments of the ink compositions described herein, the one or more matrix components is hydroxypropylmethylcellulose. In some embodiments, hydroxypropylmethylcellulose is present in an amount of about 0.1% to about 8%, about 0.1% to about 6%, about 0.1% to about 4%, about 0.1% to about 3%, about 0.1% to about 2%, about 0.1% to about 1%, about 8% to about 10%, about 6% to about 10%, about 4% to about 10%, about 2% to about 10%, about 1% to about 5%, about 1% to about 4%, about 1% to about 3%, about 1% to about 2%, about 2% to about 5%, about 2% to about 4%, or about 3% to about 5% w/w of the composition. In some embodiments, hydroxypropylmethylcellulose is present in an amount of about 0.1%, about 0.5%, about 1%, about 1.5%, about 2%, about 2.5%, about 3%, about 3.5%, about 4%, about 4%, about 4.5%, about 5%, about 5.5%, about 6%, about 7%, about 8%, about 9%, or about 10% w/w of the composition.

In some embodiments of the ink compositions described herein, the composition further comprises one or more skin penetration enhancers. “Skin penetration enhancer” as used herein refers to a substance that penetrates (penetrant) into skin to reversibly decrease the barrier resistance. In some embodiments of the ink compositions described herein, a skin penetration enhancer can also enhance the solubility of the penetrant to increase loading, which may, for example, enhance the flux of the penetrant across the skin. Non-limiting examples of a skin penetration enhancer include an alcohol, an amide, an ester, an ether alcohol, a fatty acid, a glycol, a pyrrolidone, a sulphoxide, a surfactant, and a terpene. For example, a skin penetration enhancer can include ethanol, isopropanol, decanol, hexanol, lauryl alcohol, myristyl alcohol, octanol, octyl dodecanol, oleyl alcohol, azone, ethyl acetate, octyl salicylate, padimate O, ethyl oleate, glyceryl monoleate, glyceryl monocaprate, glyceryl tricaprylate, isopropyl myristate, isopropyl palmitate, propylene glycol monolaurate, propylene glycol monocaprylate, diethylene glycol monoethyl ether (TRANSCUTOL®), lauric acid, linoleic acid, linolenic acid, myristic acid, oleic acid, palmitic acid, stearic acid, isostearic acid, dipropylene glycol, propylene glycol, 1,2-butylene glycol, 1,3-butylene glycol, n-methyl-2-pyrrolidone, 2-pyrrolidone, decylmethyl sulphoxide, dimethyl sulphoxide, sodium lauryl sulphate, alkyl dimethylbenzyl ammonium halides, alkyl trimethyl ammonium halides, alkyl pyridinium halides, brij 36t, polyethylene glycol sorbitan monooleate (e.g., TWEEN® 80), eugenol, D-limonene, menthol, menthone, farnesol, and neridol. See, for example, Lane, Int. J. Pharm. 447:12-21, 2013, which is herein incorporated by reference in its entirety.

In some embodiments of the ink compositions described herein, the one or more skin penetration enhancers are present in an amount of about 0.1% to about 50% w/w of the composition. For example, about 0.1% to about 25%, about 25% to about 50%, or about 15% to about 35% w/w of the composition. In some embodiments, the one or more skin penetration enhancers are present in an amount of about 0.1% to about 10% w/w of the composition. In some embodiments of the ink compositions described herein, the surface-active agent is present in an amount of about 0.5% to about 5% w/w of the composition. For example, about 0.5% to about 5%, about 0.5% to about 4%, about 0.5% to about 3%, about 0.5% to about 2%, about 2% to about 5%, about 2% to about 4%, or about 3% to about 5% w/w of the composition. In some embodiments of the ink compositions described herein, the one or more skin penetration enhancers are present in an amount of about 0.5%, about 1%, about 1.5%, about 2%, about 2.5%, about 3%, about 3.5%, about 4%, about 4%, about 4.5%, about 5%, about 5.5%, about 6%, about 7%, about 8%, about 9%, or about 10% w/w of the composition.

In some embodiments of the ink compositions described herein, the one or more skin penetration enhancers are selected from the group consisting of: an alcohol, an amide, an ester, an ether alcohol, a fatty acid, a glycol, a pyrrolidone, a sulphoxide, a surfactant, and a terpene. In some embodiments, the one or more skin penetration enhancers are selected from the group consisting of: ethanol, isopropanol, decanol, hexanol, lauryl alcohol, myristyl alcohol, octanol, octyl dodecanol, oleyl alcohol, azone, ethyl acetate, octyl salicylate, padimate O, ethyl oleate, glyceryl monoleate, glyceryl monocaprate, glyceryl tricaprylate, isopropyl myristate, isopropyl palmitate, propylene glycol monolaurate, propylene glycol monocaprylate, diethylene glycol monoethyl ether (TRANSCUTOL®), lauric acid, linoleic acid, linolenic acid, myristic acid, oleic acid, palmitic acid, stearic acid, isostearic acid, dipropylene glycol, propylene glycol, 1,2-butylene glycol, 1,3-butylene glycol, n-methyl-2-pyrrolidone, 2-pyrrolidone, decylmethyl sulphoxide, dimethyl sulphoxide, sodium lauryl sulphate, alkyl dimethylbenzyl ammonium halides, alkyl trimethyl ammonium halides, alkyl pyridinium halides, brij 36t, polyethylene glycol sorbitan monooleate (e.g., TWEEN® 80), eugenol, D-limonene, menthol, menthone, farnesol, and neridol.

In some embodiments of the ink compositions described herein, the one or more skin penetration enhancers are selected from the group consisting of: an alcohol, an amide, an ester, an ether alcohol, a fatty acid, a glycol, a pyrrolidone, a sulphoxide, a surfactant, and a terpene. In some embodiments, the one or more skin penetration enhancers are selected from the group consisting of: ethanol, isopropanol, decanol, hexanol, lauryl alcohol, myristyl alcohol, octanol, octyl dodecanol, oleyl alcohol, azone, ethyl acetate, octyl salicylate, padimate O, ethyl oleate, glyceryl monoleate, glyceryl monocaprate, glyceryl tricaprylate, isopropyl myristate, isopropyl palmitate, propylene glycol monolaurate, propylene glycol monocaprylate, lauric acid, linoleic acid, linolenic acid, myristic acid, oleic acid, palmitic acid, stearic acid, isostearic acid, dipropylene glycol, propylene glycol, 1,2-butylene glycol, 1,3-butylene glycol, n-methyl-2-pyrrolidone, 2-pyrrolidone, decylmethyl sulphoxide, dimethyl sulphoxide, sodium lauryl sulphate, alkyl dimethylbenzyl ammonium halides, alkyl trimethyl ammonium halides, alkyl pyridinium halides, brij 36t, polyethylene glycol sorbitan monooleate (e.g., TWEEN® 80), eugenol, D-limonene, menthol, menthone, farnesol, and neridol.

In some embodiments of the ink compositions described herein, the composition further comprises one or more temporary colorants. In some embodiments of the ink compositions described herein, the temporary colorant can be used to show what the semi-permanent tattoo will look like, outline the location of the semi-permanent tattoo on an applicator, provide a logo, e.g., brand logo, that does not transfer to the skin or transfers but is present for a short time as compared to the duration of the semi-permanent tattoo. In some embodiments of the ink compositions described herein, the temporary colorant can be used to provide a changing semi-permanent tattoo that changes appearance with time, e.g., with semi-permanent colorant duration and temporary colorant duration. Non-limiting examples of temporary colorants include iron oxide black (Fe₃O₄), iron oxide (FeO), carbon, logwood, ochre, cinnabar (HgS), cadmium red (CdSe), iron (III) oxide (Fe₂O₃), naphthol-AS pigment, disazodiarylide, disazopyrazolone, cadmium seleno-sulfide, cadmium yellow (CdS, CdZnS), curcuma yellow, chrome yellow (PbCrO₄), disazodiarylide, chromium oxide (Cr₂O₃), malachite [Cu₂(CO₃)(OH)₂], a ferrocyanide, a ferricyanide, lead chromate, monoazo pigment, Cu/Al phthalocyanine, Cu phthalocyanine, azure blue, cobalt blue, Cu-phthalocyanine, manganese violet (manganese ammonium pyrophosphate), an aluminum salt, quinacridone, dioxazine/carbazole, lead white (lead carbonate), titanium dioxide (TiO₂), barium sulfate (BaSO₄), zinc oxide, anthraquinone dyes and derivatives, annatto, caramel, ß-carotene, bismuth citrate, disodium EDTA copper, potassium sodium copper chlorophyllin, dihydroxyacetone, bismuth oxychloride, guaiazulene, henna, ferric ammonium ferrocyanide, ferric ferrocyanide, chromium hydroxide green, chromium oxide greens, guanine, lead acetate, pyrophillite, mica, silver, titanium dioxide, aluminum powder, bronze powder, copper powder, ultramarines, manganese violet, zinc oxide, luminescent zinc sulfide, D&C Black Nos. 2 and 3, FD&C Blue No. 1 (e.g., acid blue 9), D&C Blue No. 4, D&C Brown No. 1, FD&C Green No. 3, D&C Green Nos. 5, 6, and 8, D&C Orange Nos. 4, 5, 10 and 11, FD&C Red Nos. 4, D&C Red Nos. 6, 7, 17, 21, 22, 27, 28, 30, 32, 33, 34, 36 and 40, D&C Violet No. 2, Ext. D&C Violet No. 2, FD&C Yellow Nos. 5 and 6, D&C Yellow Nos. 7, 8, 10 and 11, and Ext. D&C Yellow No. 7. In some embodiments, the temporary colorant does not contain an amine group. In some embodiments, the temporary colorant does not contain an amine group.

In some embodiments of the ink compositions described herein, the one or more temporary colorants are present in an amount of about 0.01% to about 5% w/w of the composition. For example, about 0.01% to about 5%, about 0.01% to about 4%, about 0.01% to about 3%, about 0.01% to about 2%, about 0.01% to about 1%, about 0.01% to about 0.5%, about 0.5% to about 5%, about 1% to about 5%, about 2% to about 5%, about 3% to about 5% w/w, about 4% to about 5%, or about 2% to about 4% w/w of the composition. In some embodiments, the one or more temporary colorants are present in an amount of about 0.06% to about 0.1%, about 0.07% to about 0.11%, about 0.08% to about 0.12%, about 0.09% to about 0.13%, about 0.1% to about 0.14%, about 0.11% to about 0.15%, about 0.12% to about 0.16%, about 0.13% to about 0.17%, about 0.14% to about 0.18%, about 0.15% to about 0.19%, about 0.16% to about 0.2%, about 0.17% to about 0.21%, or about 0.18% to about 0.22% w/w of the composition. In some embodiments, the one or more temporary colorants are present in an amount of about 0.01%, about 0.02%, about 0.03%, about 0.04%, about 0.05%, about 0.06%, about 0.07%, about 0.08%, about 0.09%, about 0.1%, about 0.11%, about 0.12%, about 0.13%, about 0.14%, about 0.15%, about 0.16%, about 0.17%, about 0.18%, about 0.19%, about 0.2%, about 0.3%, about 0.4%%, about 0.5%, about 0.6%, about 0.8%, about 1% to about 2%, about 1.5% to about 2.5%, about 2% to about 3%, about 2.5% to about 3%, about 3% to about 4%, about 3.5% to about 4.5%, or about 4% to about 5% w/w of the composition. In some embodiments of the ink compositions described herein, the one or more temporary colorants are present in an amount of about 0.01% to about 1%, about 0.01% to about 0.1%, about 0.01% to about 0.2%, about 0.01% to about 0.3%, about 0.01% to about 0.4%, about 0.01% to about 0.5%, about 0.01% to about 0.6%, about 0.01% to about 0.7%, about 0.01% to about 0.8%, about 0.01% to about 0.9%, about 0.9% to about 1%, about 0.8% to about 1%, about 0.7% to about 1%, about 0.6% to about 1%, about 0.5% to about 1%, about 0.4% to about 1%, about 0.3% to about 1%, about 0.2% to about 1%, about 0.1% to about 1% w/w of the composition. For example, about 0.1% to about 0.3%, about 0.2% to about 0.4%, or about 0.3% to about 0.5% w/w of the composition. In some embodiments of the ink compositions described herein, the one or more temporary colorants are present in an amount of about 0.01%, about 0.05%, about 0.1%, about 0.15%, about 0.2%, about 0.25%, about 0.3%, about 0.35%, about 0.4%, about 0.45%, about 0.5%, about 1%, about 1.5%, about 2%, about 2.5%, about 3%, about 3.5%, about 4%, about 4%, about 4.5%, about 5%, about 5.5%, about 6%, about 7%, about 8%, about 9%, or about 10% w/w of the composition.

In some embodiments, the one or more temporary colorants is acid blue 9 (e.g., FD&C Blue No. 1). In some embodiments, acid blue 9 is present in an amount of about 0.01% to about 5% w/w of the composition. For example, about 0.01% to about 5%, about 0.01% to about 4%, about 0.01% to about 3%, about 0.01% to about 2%, about 0.01% to about 1%, about 0.01% to about 0.5%, about 2% to about 5%, about 3% to about 5% w/w, about 4% to about 5%, or about 2% to about 4% of the composition. In some embodiments, acid blue 9 is present in an amount of about 0.01%, about 0.02%, about 0.03%, about 0.04%, about 0.05%, about 0.06%, about 0.07%, about 0.08%, about 0.09%, about 0.1%, about 0.11%, about 0.12%, about 0.13%, about 0.14%, about 0.15%, about 0.16%, about 0.17%, about 0.18%, about 0.19%, about 0.2%, about 0.3%, about 0.4%%, about 0.5%, about 0.6%, about 0.8%, about 1% to about 2%, about 1.5% to about 2.5%, about 2% to about 3%, about 2.5% to about 3%, about 3% to about 4%, about 3.5% to about 4.5%, or about 4% to about 5% w/w of the composition. In some embodiments of the ink compositions described herein, acid blue 9 is present in an amount of about 0.5%, about 1%, about 1.5%, about 2%, about 2.5%, about 3%, about 3.5%, about 4%, about 4%, about 4.5%, about 5%, about 5.5%, about 6%, about 7%, about 8%, about 9%, or about 10% w/w of the composition.

In some embodiments, the ink composition further comprises one or more preservatives. “Preservative” as used herein refers to an agent that protects against decay, discoloration, and/or spoilage. Non-limiting examples of a preservative include ascorbic acid, an ascorbate, a palmitate, citric acid, a benzoate, a benzoic acid, a propionate, propionic acid, a sorbate, sorbic acid, a salicylic acid, a salicylate, hexa-2,4-dienoic acid, a hexa-2,4-dienoate, formaldehyde, a formaldehyde releaser, formic acid and its salts, 3-acetyl-6-methylpyran-2,4-(3H)-dione and its salts, 3,3′-dibromo-4,4′-hexamethylenedioxydibenzamidine and its salts, thiomersal, phenylmercuric salts, undec-10-enoic acid and its salts, 1,3-bis (2-ethylhexyl) hexahydro-5-methyl-5-pyrimidine, 5-bromo-5-nitro-1,3-dioxane, bronopol, 2,4-dichlorobenzyl alcohol, 1-(4-chlorophenyl)-3-(3,4-dichlorophenyl) urea, chlorocresol, chloroxylenol, 5-chloro-2-(2,4-dichlorophenoxy) phenol, N,N″-methylenebis[N′-[3-(hydroxymethyl)-2,5-dioxoimidazolidin-4-yl]urea], polyaminopropyl biguanide, methenamine, quaternium-15, climbazole, DMDM hydantoin, benzyl alcohol, 1-Hydroxy-4-methyl-6-(2,4,4-trimethylpentyl)-2-pyridon, piroctone olamine, bromochlorophene, o-cymen-5-ol, chlorophene, chloroacetaminde, methylchloroisothiazolinone, methylisothiazolinone, phenoxyisopropanol, chlorhexidine, chlorhexidine diacetate, chlorhexidine digluconate, chlorhexidine dihydrochloride, dimethyl oxazolidine, behentrimonium chloride, cetri-monium bromide, cetrimonium chloride, laurtrimonium bromide, laurtrimonium chloride, steartrimonium bromide, steartrimonium chloride, diazolidinyl urea, hexamidine, hexamidine diisethionate, hexamidine paraben, glutaral, 7-ethylbicyclooxazolidine, chlorphenesin, sodium hydroxymethylglycinate, silver chloride, benzethonium chloride, benzalkonium chloride, benza-lkonium bromide, benzalkonium saccharinate, benzylhemiformal, iodopropynyl butylcarbamate, biphenyl-2-ol and its salts, pyrithionine zinc, an erythorbate, a nitrite, ethylenediaminetetraacetic acid (EDTA), butylated hydroxyanisole (BHA), butylated hydroxytoluene (BHT), capryllic acid, dilauryl thiodipropionate, erythorbic acid, gum guaiac, methylparaben, a sulfite, a bisulfite, a metabisulfite, propyl gallatepy, propylparaben, stannous chloride, sulfur dioxide, thiodipropionic acid, an isothiazoline, a paraben, phenoxyethanol, ethylhexylglycerin, a glycol, and a tocopherol.

In some embodiments of the ink compositions described herein, the one or more preservatives are present in an amount of about 0.05% to about 5% w/w of the composition. For example, about 0.05% to about 0.1%, about 0.05% to about 0.5%, about 0.05% to about 1%, about 0.05% to about 2%, about 0.05% to about 3%, about 0.05% to about 4%, about 4% to about 5%, about 3% to about 5%, about 2% to about 5%, about 1% to about 5%, about 0.5% to about 5%, or about 0.1% to about 5% w/w of the composition. In some embodiments of the ink compositions described herein, the one or more preservatives are present in an amount of 0.1% to about 2%, 0.5% to about 2.5%, about 1% to about 3%, about 1.5% to about 3.5%, about 2% to about 4%, about 2.5% to about 4.5% w/w of the composition. In some embodiments of the ink compositions described herein, the one or more preservatives are present in an amount of about 0.5% to about 2% w/w of the composition. In some embodiments of the ink compositions described herein, the one or more preservatives are present in an amount of about 0.5% to about 1.5%, about 0.5% to about 1.0%, about 1.5% to about 2%, about 1% to about 2%, about 0.5% to about 2%, or about 0.8% to about 2% w/w of the composition. For example, about 0.05% to about 0.4%, about 0.05% to about 0.2%, about 0.2% to about 0.5%, about 0.15% to about 0.25%, or about 0.1% to about 0.3% w/w of the composition. In some embodiments of the ink compositions described herein, the one or more preservatives are present in an amount of about 0.01%, about 0.05%, about 0.1%, about 0.12%, about 0.14%, about 0.15%, about 0.16%, about 0.18%, about 0.2%, about 0.22% about 0.25%, about 0.26%, about 0.28%, about 0.3%, about 0.32%, about 0.34%, about 0.35%, about 0.36%, about 0.38%, about 0.4%, about 0.45%, about 0.5%, about 0.6%, about 0.65%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, or about 2% w/w of the composition.

In some embodiments of the ink compositions described herein, the one or more preservatives are selected form the group consisting of: ascorbic acid, an ascorbate, a palmitate, citric acid, a benzoate, a benzoic acid, a propionate, propionic acid, a sorbate, sorbic acid, a salicylic acid, a salicylate, hexa-2,4-dienoic acid, a hexa-2,4-dienoate, formaldehyde, a formaldehyde releaser, formic acid and its salts, 3-acetyl-6-methylpyran-2,4-(3H)-dione and its salts, 3,3′-dibromo-4,4′-hexamethylenedioxydibenzamidine and its salts, thiomersal, phenylmercuric salts, undec-10-enoic acid and its salts, 1,3-bis (2-ethylhexyl) hexahydro-5-methyl-5-pyrimidine, 5-bromo-5-nitro-1,3-dioxane, bronopol, 2,4-dichlorobenzyl alcohol, 1-(4-chlorophenyl)-3-(3,4-dichlorophenyl) urea, chlorocresol, chloroxylenol, 5-chloro-2-(2,4-dichlorophenoxy) phenol, N,N″-methylenebis[N′-[3-(hydroxymethyl)-2,5-dioxoimidazolidin-4-yl]urea], polyaminopropyl biguanide, methenamine, quaternium-15, climbazole, DMDM hydantoin, benzyl alcohol, 1-Hydroxy-4-methyl-6-(2,4,4-trimethylpentyl)-2-pyridon, piroctone olamine, bromochlorophene, o-cymen-5-ol, chlorophene, chloroacetaminde, methylchloroisothiazolinone, methyli sothiazolinone, phenoxyisopropanol, chlorhexidine, chlorhexidine diacetate, chlorhexidine digluconate, chlorhexidine dihydrochloride, dimethyl oxazolidine, behentrimonium chloride, cetri-monium bromide, cetrimonium chloride, laurtrimonium bromide, laurtrimonium chloride, steartrimonium bromide, steartrimonium chloride, diazolidinyl urea, hexamidine, hexamidine diisethionate, hexamidine paraben, glutaral, 7-ethylbicyclooxazolidine, chlorphenesin, sodium hydroxymethylglycinate, silver chloride, benzethonium chloride, benzalkonium chloride, benza-lkonium bromide, benzalkonium saccharinate, benzylhemiformal, iodopropynyl butylcarbamate, biphenyl-2-ol and its salts, pyrithionine zinc, an erythorbate, a nitrite, ethylenediaminetetraacetic acid (EDTA), butylated hydroxyanisole (BHA), butylated hydroxytoluene (BHT), capryllic acid, dilauryl thiodipropionate, erythorbic acid, gum guaiac, methylparaben, a sulfite, a bisulfite, a metabisulfite, propyl gallatepy, propylparaben, stannous chloride, sulfur dioxide, thiodipropionic acid, an isothiazoline, a paraben, phenoxyethanol, ethylhexylglycerin, a glycols, and a tocopherol.

In some embodiments of the ink compositions described herein, the one or more preservatives are phenoxyethanol and ethylhexylglycerin (e.g., Euxyl® PE9010). In some embodiments of the ink compositions described herein, the phenoxyethanol and ethylhexylglycerin are present in the composition at a ratio of 9:1 phenoxyethanol:ethylhexylglycerin.

In some embodiments of the ink compositions described herein, the composition is configured to have a pH of about 4 to about 7. For example, about 4 to about 5, about 4.5 to about 5.5, about 5 to about 6, about 5.5 to about 6.5, or about 6 to about 7. In some embodiments, the composition is configured to have a pH of about 4, about 4.25, about 4.5, about 4.75, about 5, about 5.25, about 5.5, about 5.75, about 6, about 6.25, about 6.5, about 6.75, or about 7. In some embodiments of the ink compositions described herein, the composition is configured to have a pH of about 4 to about 6. In some embodiments of the ink compositions described herein, the composition is configured to have a pH of about 4.6.

In some embodiments of the ink compositions described herein, the composition is adjusted to a pH of about 4 to about 7. For example, about 4 to about 5, about 4.5 to about 5.5, about 5 to about 6, about 5.5 to about 6.5, or about 6 to about 7. In some embodiments, the composition is adjusted to a pH of about 4, about 4.25, about 4.5, about 4.75, about 5, about 5.25, about 5.5, about 5.75, about 6, about 6.25, about 6.5, about 6.75, or about 7. In some embodiments of the ink compositions described herein, the composition is adjusted to a pH of about 4 to about 6. In some embodiments of the ink compositions described herein, the composition is adjusted to a pH of about 4.6.

In some embodiments of the ink compositions described herein, the composition has a pH of about 4 to about 7. For example, about 4 to about 5, about 4.5 to about 5.5, about 5 to about 6, about 5.5 to about 6.5, or about 6 to about 7. In some embodiments, the composition has a pH of about 4, about 4.25, about 4.5, about 4.75, about 5, about 5.25, about 5.5, about 5.75, about 6, about 6.25, about 6.5, about 6.75, or about 7. In some embodiments of the ink compositions described herein, the composition has a pH of about 4 to about 6. In some embodiments of the ink compositions described herein, the composition has a pH of about 4.6.

In some embodiments of the ink compositions described herein, phenoxyethanol and ethylhexylglycerin are present in an amount of about 0.05% to about 5% w/w of the composition. For example, about 0.05% to about 0.1%, about 0.05% to about 0.5%, about 0.05% to about 1%, about 0.05% to about 2%, about 0.05% to about 3%, about 0.05% to about 4%, about 4% to about 5%, about 3% to about 5%, about 2% to about 5%, about 1% to about 5%, about 0.5% to about 5%, or about 0.1% to about 5% w/w of the composition. In some embodiments of the ink compositions described herein, phenoxyethanol and ethylhexylglycerin are present in an amount of 0.1% to about 2%, 0.5% to about 2.5%, about 1% to about 3%, about 1.5% to about 3.5%, about 2% to about 4%, about 2.5% to about 4.5% w/w of the composition. In some embodiments of the ink compositions described herein, phenoxyethanol and ethylhexylglycerin are present in an amount of about 0.5% to about 2% w/w of the composition. In some embodiments of the ink compositions described herein, phenoxyethanol and ethylhexylglycerin are present in an amount of about 0.5% to about 1.5%, about 0.5% to about 1.0%, about 0.05% to about 0.5%, about 0.05% to about 0.1%, about 1.5% to about 2%, about 1% to about 2%, about 0.5% to about 2%, or about 0.8% to about 2%, or about 0.1% to about 0.9% w/w of the composition. For example, about 0.05% to about 0.4%, about 0.05% to about 0.2%, about 0.2% to about 0.5%, about 0.15% to about 0.25%, or about 0.1% to about 0.3% w/w of the composition. In some embodiments of the ink compositions described herein, phenoxyethanol and ethylhexylglycerin are present in an amount of about 0.01%, about 0.05%, about 0.1%, about 0.12%, about 0.14%, about 0.15%, about 0.16%, about 0.18%, about 0.2%, about 0.22% about 0.25%, about 0.26%, about 0.28%, about 0.3%, about 0.32%, about 0.34%, about 0.35%, about 0.36%, about 0.38%, about 0.4%, about 0.45%, about 0.5%, about 0.6%, about 0.65%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, or about 2% w/w of the composition.

In some embodiments, the ink composition comprises:

genipin present in an amount of about 5% to about 10% w/w of the composition;

one or more volatile solvents present in an amount of about 65% to about 80% w/w of the composition;

one or more semi-volatile semi-permanent colorant solubilizers present in an amount of about 5% to about 25% w/w of the composition. In some embodiments, the ink composition further comprises one or more surface-active agents present in an amount of about 1% to about 3% w/w of the composition. In some embodiments, the ink composition further comprises one or more surface-active agents present in an amount of about 2% w/w of the composition. In some embodiments, the ink composition further comprises one or more matrix components present in an amount of about 1% to about 3% w/w of the composition. In some embodiments, the ink composition further comprises one or more matrix components present in an amount of about 1% to about 2% w/w of the composition. In some embodiments, the composition further comprises one or more preservatives present in an amount of about 0.1% to about 2% w/w of the composition. In some embodiments, the composition further comprises one or more preservatives present in an amount of about 1% w/w of the composition. In some embodiments, the ink composition further comprises one or more temporary colorants present in an amount of about 0.1% to about 0.5% w/w of the composition. In some embodiments, the ink composition further comprises one or more temporary colorants present in an amount of about 0.3% w/w of the composition.

In some embodiments, the ink composition comprises:

genipin present in an amount of about 7% w/w of the composition;

one or more volatile solvents present in an amount of about 65% to about 80% w/w of the composition;

one or more semi-volatile semi-permanent colorant solubilizers present in an amount of about 5% to about 25% w/w of the composition. In some embodiments, the ink composition further comprises one or more surface-active agents present in an amount of about 1% to about 3% w/w of the composition. In some embodiments, the ink composition further comprises one or more surface-active agents present in an amount of about 2% w/w of the composition. In some embodiments, the ink composition further comprises one or more matrix components present in an amount of about 1% to about 3% w/w of the composition. In some embodiments, the ink composition further comprises one or more matrix components present in an amount of about 1% to about 2% w/w of the composition. In some embodiments, the composition further comprises one or more preservatives present in an amount of about 0.1% to about 2% w/w of the composition. In some embodiments, the composition further comprises one or more preservatives present in an amount of about 1% w/w of the composition. In some embodiments, the ink composition further comprises one or more temporary colorants present in an amount of about 0.1% to about 0.5% w/w of the composition. In some embodiments, the ink composition further comprises one or more temporary colorants present in an amount of about 0.3% w/w of the composition.

In some embodiments, the ink composition comprises:

genipin present in an amount of about 7% w/w of the composition;

one or more volatile solvents present in an amount of about 70% to about 75% w/w of the composition;

one or more semi-volatile semi-permanent colorant solubilizers present in an amount of about 15% w/w of the composition. In some embodiments, the ink composition further comprises one or more surface-active agents present in an amount of about 1% to about 3% w/w of the composition. In some embodiments, the ink composition further comprises one or more surface-active agents present in an amount of about 2% w/w of the composition. In some embodiments, the ink composition further comprises one or more matrix components present in an amount of about 1% to about 3% w/w of the composition. In some embodiments, the ink composition further comprises one or more matrix components present in an amount of about 1% to about 2% w/w of the composition. In some embodiments, the composition further comprises one or more preservatives present in an amount of about 0.1% to about 2% w/w of the composition. In some embodiments, the composition further comprises one or more preservatives present in an amount of about 1% w/w of the composition. In some embodiments, the ink composition further comprises one or more temporary colorants present in an amount of about 0.1% to about 0.5% w/w of the composition. In some embodiments, the ink composition further comprises one or more temporary colorants present in an amount of about 0.3% w/w of the composition.

In some embodiments, the ink composition comprises:

genipin present in an amount of about 7% w/w of the composition;

one or more volatile solvents present in an amount of about 73% w/w of the composition;

one or more semi-volatile semi-permanent colorant solubilizers present in an amount of about 15% w/w of the composition. In some embodiments, the ink composition further comprises one or more surface-active agents present in an amount of about 1% to about 3% w/w of the composition. In some embodiments, the ink composition further comprises one or more surface-active agents present in an amount of about 2% w/w of the composition. In some embodiments, the ink composition further comprises one or more matrix components present in an amount of about 1% to about 3% w/w of the composition. In some embodiments, the ink composition further comprises one or more matrix components present in an amount of about 1% to about 2% w/w of the composition. In some embodiments, the composition further comprises one or more preservatives present in an amount of about 0.1% to about 2% w/w of the composition. In some embodiments, the composition further comprises one or more preservatives present in an amount of about 1% w/w of the composition. In some embodiments, the ink composition further comprises one or more temporary colorants present in an amount of about 0.1% to about 0.5% w/w of the composition. In some embodiments, the ink composition further comprises one or more temporary colorants present in an amount of about 0.3% w/w of the composition.

In some embodiments, the composition comprises:

genipin present in an amount of about 5% to about 10% w/w of the composition;

one or more volatile solvents present in an amount of about 65% to about 80% w/w of the composition, wherein the one or more semi-volatile solvents are water and ethanol;

one or more semi-volatile semi-permanent colorant solubilizers present in an amount of about 5% to about 25% w/w of the composition, wherein the one or more semi-volatile semi-permanent colorant solubilizers comprise diethylene glycol monoethyl ether. In some embodiments, the ink composition further comprises one or more surface-active agents present in an amount of about 1% to about 3% w/w of the composition. In some embodiments, the ink composition further comprises one or more surface-active agents present in an amount of about 2% w/w of the composition. In some embodiments, the ink composition further comprises one or more matrix components present in an amount of about 1% to about 3% w/w of the composition. In some embodiments, the ink composition further comprises one or more matrix components present in an amount of about 1% to about 2% w/w of the composition. In some embodiments, the composition further comprises one or more preservatives present in an amount of about 0.1% to about 2% w/w of the composition. In some embodiments, the composition further comprises one or more preservatives present in an amount of about 1% w/w of the composition. In some embodiments, the ink composition further comprises one or more temporary colorants present in an amount of about 0.1% to about 0.5% w/w of the composition. In some embodiments, the ink composition further comprises one or more temporary colorants present in an amount of about 0.3% w/w of the composition.

In some embodiments, the ink composition comprises:

genipin present in an amount of about 7% w/w of the composition;

one or more volatile solvents present in an amount of about 70% to about 75% w/w of the composition, wherein the one or more semi-volatile solvents are water and ethanol;

one or more semi-volatile semi-permanent colorant solubilizers present in an amount of about 5% to about 25% w/w of the composition, wherein the one or more semi-volatile semi-permanent colorant solubilizers comprise diethylene glycol monoethyl ether. In some embodiments, the ink composition further comprises one or more surface-active agents present in an amount of about 1% to about 3% w/w of the composition. In some embodiments, the ink composition further comprises one or more surface-active agents present in an amount of about 2% w/w of the composition. In some embodiments, the ink composition further comprises one or more matrix components present in an amount of about 1% to about 3% w/w of the composition. In some embodiments, the ink composition further comprises one or more matrix components present in an amount of about 1% to about 2% w/w of the composition. In some embodiments, the composition further comprises one or more preservatives present in an amount of about 0.1% to about 2% w/w of the composition. In some embodiments, the composition further comprises one or more preservatives present in an amount of about 1% w/w of the composition. In some embodiments, the ink composition further comprises one or more temporary colorants present in an amount of about 0.1% to about 0.5% w/w of the composition. In some embodiments, the ink composition further comprises one or more temporary colorants present in an amount of about 0.3% w/w of the composition.

In some embodiments, the ink composition comprises:

genipin present in an amount of about 7% w/w of the composition;

one or more volatile solvents present in an amount of about 70% to about 75% w/w of the composition, wherein the one or more semi-volatile solvents are water and ethanol;

one or more semi-volatile semi-permanent colorant solubilizers present in an amount of about 15% w/w of the composition, wherein the one or more semi-volatile semi-permanent colorant solubilizers comprise diethylene glycol monoethyl ether. In some embodiments, the ink composition further comprises one or more surface-active agents present in an amount of about 1% to about 3% w/w of the composition. In some embodiments, the ink composition further comprises one or more surface-active agents present in an amount of about 2% w/w of the composition. In some embodiments, the ink composition further comprises one or more matrix components present in an amount of about 1% to about 3% w/w of the composition. In some embodiments, the ink composition further comprises one or more matrix components present in an amount of about 1% to about 2% w/w of the composition. In some embodiments, the composition further comprises one or more preservatives present in an amount of about 0.1% to about 2% w/w of the composition. In some embodiments, the composition further comprises one or more preservatives present in an amount of about 1% w/w of the composition. In some embodiments, the ink composition further comprises one or more temporary colorants present in an amount of about 0.1% to about 0.5% w/w of the composition. In some embodiments, the ink composition further comprises one or more temporary colorants present in an amount of about 0.3% w/w of the composition.

In some embodiments, the ink composition comprises:

genipin present in an amount of about 7% w/w of the composition;

one or more volatile solvents present in an amount of about 73% w/w of the composition, wherein the one or more semi-volatile solvents are water and ethanol;

one or more semi-volatile semi-permanent colorant solubilizers present in an amount of about 15% w/w of the composition, wherein the one or more semi-volatile semi-permanent colorant solubilizers comprise diethylene glycol monoethyl ether. In some embodiments, the ink composition further comprises one or more surface-active agents present in an amount of about 1% to about 3% w/w of the composition. In some embodiments, the ink composition further comprises one or more surface-active agents present in an amount of about 2% w/w of the composition. In some embodiments, the ink composition further comprises one or more matrix components present in an amount of about 1% to about 3% w/w of the composition. In some embodiments, the ink composition further comprises one or more matrix components present in an amount of about 1% to about 2% w/w of the composition. In some embodiments, the composition further comprises one or more preservatives present in an amount of about 0.1% to about 2% w/w of the composition. In some embodiments, the composition further comprises one or more preservatives present in an amount of about 1% w/w of the composition. In some embodiments, the ink composition further comprises one or more temporary colorants present in an amount of about 0.1% to about 0.5% w/w of the composition. In some embodiments, the ink composition further comprises one or more temporary colorants present in an amount of about 0.3% w/w of the composition.

In some embodiments, the one or more surface-active agents is 1,2-hexanediol. In some embodiments, the one or more matrix components is trehalose. In some embodiments, the one or more preservatives are phenoxyethanol and ethylhexylglycerin (e.g., Euxyl® PE9010). In some embodiments, the phenoxyethanol and ethylhexylglycerin are present in the composition at a ratio of 9:1 phenoxyethanol:ethylhexylglycerin. In some embodiments, the one or more temporary colorants is acid blue 9.

In some embodiments, the viscosity of the ink composition is about 3 to about 6 centipoise. In some embodiments, the viscosity of the ink composition is about 3.7 to about 4.1 centipoise. In some embodiments, the viscosity of the ink composition is about 3.8 centipoise.

In some embodiments, the dynamic surface tension of the ink composition is about 25 to about 30 dynes/cm. In some embodiments, the dynamic surface tension of the ink composition is about 25 to about 26 dynes/cm. In some embodiments, the dynamic surface tension of the ink composition is about 26 dynes/cm.

In some embodiments, the pH of the inkjet printer ink composition is about 4.5 to about 5. In some embodiments, the pH of the ink composition is about 4.9. In some embodiments, the pH of the inkjet printer ink composition is about 4.6.

In some embodiments of the ink compositions described herein, some of the components described above may be satisfied by a single component having multiple purposes. For example, in some embodiments, a skin penetration enhancer can also act as a semi-volatile semi-permanent colorant solubilizer. Where indicated, however, if a component is listed separately in a specific embodiment of a formulation, it is intended that each component listed is a separate component in the composition. For example, a composition comprising one or more semi-permanent colorants; one or more volatile solvents; one or more semi-volatile semi-permanent colorant solubilizers; and one or more skin penetration enhancers comprises at least 4 separate components, however, it is still possible that each of the separate components can serve multiple and sometimes overlapping purposes.

In some embodiments of the ink compositions described herein, the composition is an inkjet printer ink composition configured to be printable using an inkjet printer. In some embodiments, the composition is amine-free. For example, the composition comprises less than about 1% amines, e.g., less than about 0.8%, 0.05%, 0.02%, or about 0.01% w/w of the composition.

Also provided herein are inkjet printer ink compositions comprising purified genipin; one or more volatile solvents; and one or more semi-volatile semi-permanent colorant solubilizers. In some embodiments, the inkjet printer ink composition comprises:

purified genipin present in an amount of about 7% to about 11% w/w of the composition;

one or more volatile solvents present in an amount of about 60% to about 80% w/w of the composition, wherein the one or more semi-volatile solvents are water and ethanol;

one or more semi-volatile semi-permanent colorant solubilizers present in an amount of about 10% to about 30% w/w of the composition, wherein the one or more semi-volatile semi-permanent colorant solubilizers comprise diethylene glycol monoethyl ether;

one or more surface-active agents present in an amount of about 0.25% to about 5% w/w of the composition;

one or more matrix components present in an amount of about 0.1% to about 5% w/w of the composition.

In some embodiments, an inkjet printer ink composition can comprise:

purified genipin present in an amount of about 7% to about 11% w/w of the composition;

one or more volatile solvents present in an amount of about 65% to about 75% w/w of the composition, wherein the one or more semi-volatile solvents are water and ethanol;

one or more semi-volatile semi-permanent colorant solubilizers present in an amount of about 15% to about 25% w/w of the composition, wherein the one or more semi-volatile semi-permanent colorant solubilizers comprise diethylene glycol monoethyl ether;

one or more surface-active agents present in an amount of about 0.25% to about 5% w/w of the composition;

one or more matrix components present in an amount of about 0.5% to about 3.5% w/w of the composition.

In some embodiments, an inkjet printer ink composition can further comprise one or more of a skin penetration enhancer, a temporary colorant, and a preservative.

In some embodiments, the inkjet printer ink composition is configured to have a pH of about 4 to about 7. For example, about 4 to about 5, about 4.5 to about 5.5, about 5 to about 6, about 5.5 to about 6.5, or about 6 to about 7. In some embodiments, the inkjet printer ink composition has a pH of about 4, about 4.25, about 4.5, about 4.75, about 5, about 5.25, about 5.5, about 5.75, about 6, about 6.25, about 6.5, about 6.75, or about 7.

In some embodiments, the inkjet printer ink composition has a pH of about 4 to about 7. For example, about 4 to about 5, about 4.5 to about 5.5, about 5 to about 6, about 5.5 to about 6.5, or about 6 to about 7. In some embodiments, the inkjet printer ink composition has a pH of about 4, about 4.25, about 4.5, about 4.75, about 5, about 5.25, about 5.5, about 5.75, about 6, about 6.25, about 6.5, about 6.75, or about 7.

In some embodiments, the composition is amine-free. For example, the inkjet printer ink composition comprises less than about 1% amines, e.g., less than about 0.8%, about 0.05%, about 0.02%, about 0.01%, or about 0.005% w/w of the composition.

In some embodiments, an inkjet printer ink composition comprises:

purified genipin present in an amount of about 9% w/w of the composition;

one or more volatile solvents present in an amount of about 67% w/w of the composition, wherein the one or more semi-volatile solvents are water and ethanol;

one or more semi-volatile semi-permanent colorant solubilizers present in an amount of about 21% w/w of the composition, wherein the one or more semi-volatile semi-permanent colorant solubilizers comprise diethylene glycol monoethyl ether;

one or more surface-active agents present in an amount of about 1% w/w of the composition, wherein the one or more surface active agents is PEG 400; and

one or more matrix components present in an amount of about 2% w/w of the composition, wherein the one or more matrix components is trehalose. In some embodiments, the viscosity of the inkjet printer ink composition is about 4.5 centipoise. In some embodiments, the dynamic surface tension of the inkjet printer ink composition is about 30 dynes/cm. In some embodiments, the pH of the inkjet printer ink composition is about 4.9.

In some embodiments, an inkjet printer ink composition comprises:

purified genipin present in an amount of about 9% w/w of the composition;

one or more volatile solvents present in an amount of about 67% w/w of the composition, wherein the one or more semi-volatile solvents are water and ethanol;

one or more semi-volatile semi-permanent colorant solubilizers present in an amount of about 21% w/w of the composition, wherein the one or more semi-volatile semi-permanent colorant solubilizers comprise diethylene glycol monoethyl ether;

one or more surface-active agents present in an amount of about 1% w/w of the composition, wherein the one or more surface active agents is PEG 400;

one or more matrix components present in an amount of about 2% w/w of the composition, wherein the one or more matrix components is trehalose; and

one or more colorants present in an amount of about 0.2% w/w of the composition, wherein the one or more temporary colorants is FD&C Blue No. 1. In some embodiments, the viscosity of the inkjet printer ink composition is about 4.5 centipoise. In some embodiments, the dynamic surface tension of the inkjet printer ink composition is about 30 dynes/cm. In some embodiments, the pH of the inkjet printer ink composition is about 4.9.

In some embodiments, an inkjet printer ink composition comprises:

purified genipin present in an amount of about 4.5% w/w of the composition;

one or more volatile solvents present in an amount of about 70% w/w of the composition, wherein the one or more semi-volatile solvents are water and ethanol;

one or more semi-volatile semi-permanent colorant solubilizers present in an amount of about 22.5% w/w of the composition, wherein the one or more semi-volatile semi-permanent colorant solubilizers comprise diethylene glycol monoethyl ether;

one or more surface-active agents present in an amount of about 1% w/w of the composition, wherein the one or more surface active agents is PEG 400;

one or more matrix components present in an amount of about 2% w/w of the composition, wherein the one or more matrix components is trehalose; and

one or more temporary colorants present in an amount of about 0.1% w/w of the composition, wherein the one or more temporary colorants is FD&C Blue No. 1. In some embodiments, the viscosity of the inkjet printer ink composition is about 4.5 centipoise. In some embodiments, the dynamic surface tension of the inkjet printer ink composition is about 30 dynes/cm. In some embodiments, the pH of the inkjet printer ink composition is about 4.9. Also provided herein are applicator articles comprising a composition as described in any of the above embodiments; an adhesive; and an adhesive backing.

In some embodiments, an adhesive applicator article described herein can further comprise a non-adhesive liner.

In some embodiments, an adhesive applicator article can further comprise, or can be configured for use with, a heat pad.

In some embodiments of an adhesive applicator article described herein, an ink composition as described in any of the above embodiments is disposed on and/or coupled to at least a portion of the adhesive. In some embodiments of an adhesive applicator article described herein, the ink composition is dried on the applicator article. For example, after the composition is printed onto the adhesive, it can be dried by heating. In some examples, the material can be heated and/or dried using an oven, a dual convection oven, a conveyor, and/or an infrared oven. In some embodiments, the composition is dried at about 40° C. to about 80° C. For example, about 40° C. to about 50° C., about 45° C. to about 55° C., about 50° C. to about 60° C., about 55° C. to about 65° C., about 60° C. to about 70° C., about 65° C. to about 75° C., or about 70° C. to about 80° C. In some embodiments, the composition is dried at about 50° C., about 55° C., about 60° C., about 65° C., about 70° C., or about 75° C. In some embodiments, the composition is dried for a period of time. For example, the composition can be dried for about 0.25 to about 3 hours. In some embodiments, the composition is dried for about 0.25 hours to about 0.75 hours, about 0.5 hours to about 1 hour, about 0.75 hours to about 1.25 hours, about 1 hour to about 1.5 hours, about 1.25 hours to about 1.75 hours, about 1.5 hours to about 2 hours, about 1.75 hours to about 2.25 hours, about 2 hours to about 2.5 hours, about 2.25 hours to about 2.75 hours, or about 2.5 hours to about 3 hours. In some embodiments, the composition is dried for about 0.5 hours, about 1 hour, about 1.5 hours, about 2 hours, about 2.5 hours, or about 3 hours. In some embodiments, the composition is dried for about 1.25 hours to about 1.75 hours at about 55° C. to about 65° C. In some embodiments, the composition is dried for about 1.5 hours at about 60° C. In some embodiments, the composition is dried for about 1 hour at about 60° C.

In some embodiments, an adhesive applicator article as described herein can further comprise a medicament or cosmetic for delivery to a subject. In some embodiments, the adhesive applicator article can comprise a patch for delivering a medicament or a cosmetic. In some embodiments, the composition is printed on a patch.

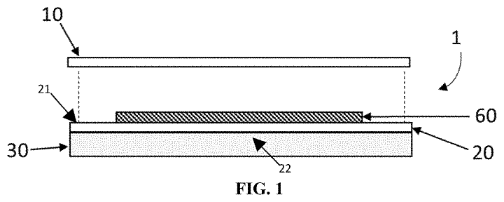

FIG. 1 illustrates an adhesive applicator article 1 according to one embodiment of the present disclosure. As shown in FIG. 1, in some embodiments, the adhesive applicator article comprises an adhesive layer 20; an adhesive backing 30; and an ink composition 60 for example, an ink composition as described in the above embodiments. In some embodiments, the adhesive applicator article optionally further comprises a liner 10. Liner 10 can be, e.g., a protective liner and/or a nonstick liner.

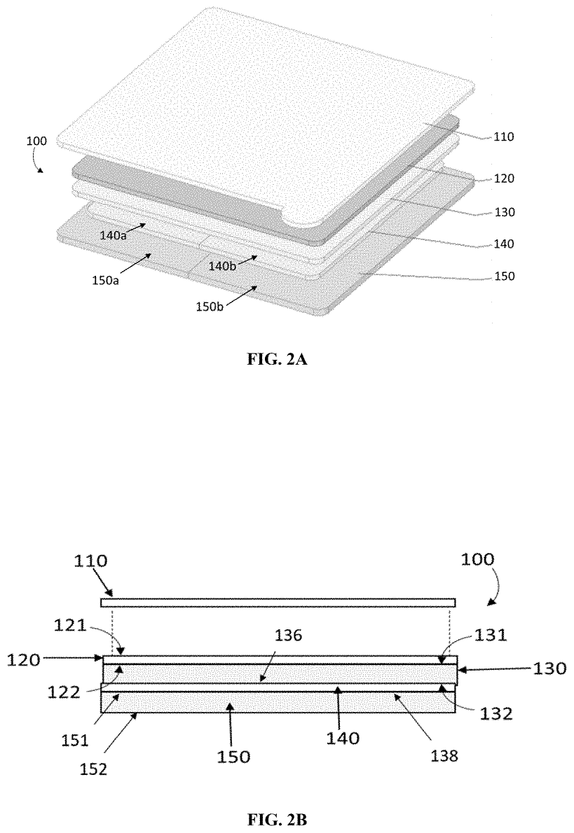

The adhesive layer 20 can include a first surface 21 and a second surface 22 opposite the first surface. In the illustrated embodiment, the adhesive layer 20 is shown with its second surface 22 disposed on and/or coupled to a first surface of the adhesive backing 30. The first surface 21 and the second surface 22 of the adhesive layer 20 are described further herein in connection with FIG. 2B, additionally, the first surface and a second surface of the adhesive backing 30 are described further herein in connection with FIG. 2B.

The ink composition 60 as described above is deposited on and/or coupled to the first surface 21 of the adhesive layer 20. In some embodiments, the ink composition 60 is deposited on and/or coupled to a portion of the first surface 21 of the adhesive layer 20. In some embodiments, the ink composition 60 is printed, e.g., via inkjet printing, onto the first surface 21 of the adhesive layer 20. In some embodiments, wherein the ink composition 60 is deposited on and/or coupled to a portion of the first surface 21 of the adhesive layer 20, the ink composition 60 is printed onto the first surface 21 of the adhesive layer 20 in any number of designs. For example, the ink composition 60 can be printed as a pattern, a shape, words, a picture, an image or art created by a user, or combinations of two or more thereof. In some embodiments, multiple layers of the ink composition 60 can be printed onto the first surface 21 of the adhesive layer 20 to create a desired pattern and/or effect.

In some optional embodiments, wherein the adhesive applicator article further comprises a liner 10, the liner 10 is deposited on and/or coupled to the ink composition 60. In some embodiments, wherein the adhesive applicator article further comprises a liner 10, the liner 10 is deposited on and/or coupled to the ink composition 60 and the first surface 21 of the adhesive layer 20, wherein the ink composition 60 is deposited on and/or coupled to a portion of the first surface 21 of the adhesive layer 20. In some embodiments, the liner 10 is a non-adhesive liner.

In some embodiments, the adhesive applicator article 1 is configured to provide proper structure for printing, manufacturing, storage, and/or application of a composition comprising a semi-permanent colorant such as those described herein. In some embodiments, the adhesive applicator article 1 is configured to provide proper structure to eliminate or reduce the risk of the adhesive folding in on itself, e.g., during application, and/or to improve the ease of application of the applicator article to a surface, e.g., skin surface, by a user.

In some embodiments, the adhesive applicator article 1 optionally further comprises a heat pad. In some embodiments, the heat pad is adhered to the second surface of the adhesive backing 30. In some embodiments, the heat pad is not attached to the adhesive applicator article 1 and is placed by a user onto the second surface of the adhesive backing 30 during application of the ink composition 60 to skin.

FIG. 2A illustrates an exploded perspective view of adhesive applicator article 100 according to another embodiment of the present disclosure. FIG. 2B illustrates a side view of the adhesive applicator article 100. As shown in FIG. 2A, in some embodiments, the adhesive applicator article 100 comprises a first adhesive layer 120; a first backing 130 (which can be analogous to the adhesive backing 30 described in connection with FIG. 1); a second adhesive layer 140, and a second backing 150. In some embodiments, the adhesive applicator article 100 optionally further comprises a liner 110. In some embodiments, the adhesive applicator article 100 optionally further comprises an ink composition, e.g., an ink composition as described above.

As illustrated by FIG. 2B, the first adhesive layer 120 can include a first surface 121 and a second surface 122 opposite the first surface 121. The first adhesive layer 120 is shown with its second surface 122 as disposed on and/or coupled to the first surface 131 of the first backing 130. The second surface 132 of the first backing 130 can be disposed on and/or coupled to the first surface 136 of the second adhesive layer 140. A second surface 138 of the second adhesive layer 140 is shown as disposed on and/or coupled to the first surface 151 of the second backing 150, where the first surface 151 of the second backing 150 is opposite the second surface 152 of the second backing 150.

In some embodiments, the first adhesive layer 120 can optionally have a backing disposed on and/or coupled to its second surface 122. In some embodiments, the first backing 130 can optionally have an adhesive layer disposed on and/or coupled to its first surface 131, its second surface 132, or both. In some embodiments, wherein the first backing 130 has an adhesive layer disposed on and/or coupled to its first surface 131, and its second surface 132, the adhesive layer disposed on and/or coupled to its first surface 131 can be a stronger adhesive than adhesive layer disposed on and/or coupled to its second surface 132. For example, in some embodiments, the weaker peel strength is adhered to the first adhesive layer 120 and the stronger adhesive is adhered to the second backing 150 such that the second backing 150 is easily removed without leaving a residue. In some embodiments, the second adhesive layer 140 can optionally have a backing disposed on and/or coupled to its first surface 136, its second surface 138, or both, as depicted in FIG. 2B. In some embodiments, the second backing 150 can optionally have an adhesive layer disposed on and/or coupled to the first surface 151. In some embodiments, wherein the second backing 150 has an adhesive layer disposed on and/or coupled to a first surface 151, and a second surface 152, the second adhesive layer 140 disposed on and/or coupled to its first surface 151 can be a stronger adhesive than an adhesive layer disposed on and/or coupled to its second surface 152. In some embodiments, wherein the second backing 150 has an adhesive layer disposed on and/or coupled to its first surface 151, and its second surface 152, a liner can be disposed on and/or coupled to the second surface 152 of the second backing 150.

In some embodiments, an ink composition, e.g., an ink composition as described above, can be deposited on and/or coupled to at least a portion of the first surface 121 of the first adhesive layer 120. In some embodiments, the ink composition can be deposited on and/or coupled to a portion of the first surface 121 of the first adhesive layer 120. In some embodiments, the ink composition is printed onto the first surface 121 of the first adhesive layer 120. In some embodiments, the ink composition can be printed onto the first surface 121 of the first adhesive layer 120 using an inkjet printer. In some embodiments, wherein the ink composition is deposited on and/or coupled to a portion of the first surface 121 of the first adhesive layer 120, the ink composition can be printed onto the first surface 121 of the first adhesive layer 120 in any number of designs. For example, the ink composition can be printed as a pattern, a shape, words, a picture, an image or art created by a user, or combinations of two or more thereof. Although examples of a pattern, a word, a picture, an image, and/or art created by a user are mentioned herein, embodiments are not so limited. For example, solid gradients of shapes (e.g., substantially solid ink distribution), variant gradients of shapes, various thicknesses of lines, among other effects can be printed. In some embodiments, multiple layers of the ink composition can be printed onto the first surface 121 of the first adhesive layer 120 to create a desired pattern and/or effect.

In some embodiments, the second adhesive 140 and second backing 150 are disposed on and/or coupled to at least a portion of the second surface 132 of the first backing 130. In some embodiments, a portion of a surface can include a perimeter, a portion of a perimeter, a grid, a section, and the like.

As illustrated in FIG. 2A, in some embodiments, each layer can optionally be composed of multiple parts or portions. For example, the second adhesive 140 and second backing 150 can be divided into two or more parts, e.g., part 140a and part 140b of the second adhesive 140, and part 150a and part 150b of the second backing 150. In some embodiments, the two or more parts can have an overlapping arrangement, can abut each other, can be separated by a distance, or a combination thereof. A portion can include a perimeter, a portion of a perimeter, a grid, a section, and the like.

In some embodiments, wherein the applicator article 100 further comprises a liner 110 and an ink composition (e.g., the ink composition 60 as described above in connection with FIG. 1), the liner 110 can be disposed on and/or coupled to the ink composition. In some embodiments, wherein the adhesive applicator article 100 further comprises a liner 110 and an ink composition as described above, the liner 110 can be disposed on and/or coupled to the ink composition and the first surface 121 of the first adhesive layer 120, wherein the ink composition is deposited on and/or coupled to a portion of the first surface 121 of the first adhesive layer 120. In some embodiments, the liner 110 is a non-adhesive liner. In some embodiments, the liner 110 can be silicone coated, fluorosilicone coated, wax coated, coated with a non-stick polymer, or similarly coated with a non-stick layer. In some embodiments, the liner 110 can be a silicone-coated paper liner.

In some embodiments, the first adhesive layer 120 can comprise an acrylic adhesive, a rubber adhesive, or other appropriate adhesive configured to receive the ink composition, allow it to dry, and allow release and penetration of at least a portion of the composition onto and/or into the skin of a user to which it is applied.

In some embodiments, the first backing 130 can be a polyurethane backing or other appropriate backing that is configured to provide, e.g., desired structure, strength, or geometry to the adhesive applicator article. In some embodiments, the first backing 130 can reduce and/or eliminate the stickiness of an adjacent adhesive layer. For example, the first backing 130 can reduce and/or eliminate the stickiness of the first adhesive layer 120. In some embodiments, the first backing 130 can increase the stiffness of the adhesive applicator article 100 during fabrication and/or use.

In some embodiments, the second adhesive layer 140 can be a laminating adhesive.

In some embodiments, the second backing 150 can be a polycarbonate backing that is configured to provide, e.g., desired structure, strength, or geometry to the adhesive applicator article 100. In some embodiments, the second backing 150 can provide structure that inhibits or prevents wrinkling of the adhesive during the application period.

In some embodiments, the adhesive applicator article 100 is configured to provide proper structure for printing, manufacturing, storage, and/or application of a composition comprising a semi-permanent colorant such as those described herein.

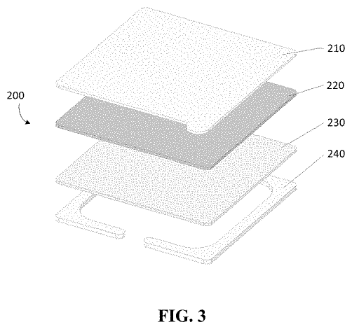

FIG. 3 illustrates an adhesive applicator article 200 according to another embodiment of the present disclosure. As shown in FIG. 3, in some embodiments, the adhesive applicator article 200 comprises an adhesive layer 220; a backing 230; and a structural layer 240. In some embodiments, the adhesive applicator article 200 optionally further comprises a liner 210. In some embodiments, the adhesive applicator article 200 optionally further comprises an ink composition (e.g., the ink composition 60 as described above in connection with FIG. 1). The adhesive layer 220 can, in some examples, be analogous to the adhesive layer 20, the adhesive layer 120, and/or the second adhesive layer 140, as described respectively in connection with FIGS. 1, 2A, and 2B. The backing 230 can, in some examples, be analogous to the adhesive backing 30, the backing 130, and/or the second backing 150 as described respectively in connection with FIGS. 1, 2A, and 2B. The liner 210 can, in some examples, be analogous to the liner 10, and/or the liner 110 as described respectively in connection with FIGS. 1, 2A, and 2B.

Although not specifically labeled in FIG. 3, as to not obstruct the examples of the disclosure, the adhesive layer 220 can include a first surface and a second surface opposite the first surface as described in connection with FIG. 2B. Although not specifically labeled in FIG. 3, as to not obstruct the examples of the disclosure, the backing 230 can include a first surface and a second surface opposite the first surface as described in connection with FIG. 2B. The adhesive layer 220 is shown with its second surface as disposed on and/or coupled to a first surface of the backing 230. A second surface of the backing 230 is disposed on and/or coupled to a first surface of the structural layer 240.

In some embodiments, the adhesive layer 220 can optionally have a backing disposed on and/or coupled to its second surface. In some embodiments, the backing 230 can optionally have an adhesive layer disposed on and/or coupled to its first surface, its second surface, or both. In some embodiments, the structural layer 240 can optionally have an adhesive layer disposed on and/or coupled to its first surface, its second surface, or both.

In some embodiments, the backing 230 is laminated onto the adhesive layer 220.

In some embodiments, an ink composition (e.g., the ink composition 60 as described above in connection with FIG. 1) is deposited on and/or coupled to at least a portion of the first surface of the adhesive layer 220. In some embodiments, the ink composition is deposited on and/or coupled to a portion of the first surface of the adhesive layer 220. In some embodiments, the ink composition is printed onto the first surface of the adhesive layer 220. In some embodiments, the ink composition can be printed onto the first surface of the adhesive layer 220 using an inkjet printer. In some embodiments, wherein the ink composition is deposited on and/or coupled to a portion of the first surface of the adhesive layer 220, the ink composition is printed onto the first surface of the adhesive layer 220 in any number of designs. For example, the ink composition can be printed as a pattern, a shape, words, a picture, an image or art created by a user, or combinations of two or more thereof. In some embodiments, multiple layers of the ink composition can be printed onto the first surface of the adhesive layer 220 to create a desired pattern and/or effect.

In some embodiments, the structural layer 240 is disposed on and/or coupled to a portion of the backing 230. In some embodiments, the liner 210 functions as an additional structural layer. For example, the liner 210 can be a framing. In some embodiments, a portion of a surface can include a perimeter, a portion of a perimeter, and a grid, a section, and the like. In some embodiments, the structural layer 240 and/or the liner 210) can be kiss cut. For example, die cut wherein the die only penetrates the structural layer. In some embodiments, the inner corners of the structural layer 240 are rounded. Rounded corners may, for example, prevent tearing during removal and/or provide an indication to the user about where to remove the applicator.

In some embodiments, one or more of the layers of the adhesive article 200 can be composed of more than one part or portion. In some embodiments, the two or more parts can have an overlapping arrangement, can abut each other, or the two or more parts can be a combination thereof. In some embodiments, the two or more parts are a portion of an adjacent layer. A portion can include a perimeter, a portion of a perimeter, and a grid.

In some embodiments, wherein the adhesive applicator article 200 optionally further comprises a liner 210 and an ink composition (e.g., an ink composition as described above in connection with FIG. 1), the liner 210 is disposed on and/or coupled to the ink composition 260. In some embodiments, wherein the adhesive applicator article further comprises a liner 210 and a composition as described above 260, the liner 210 is disposed on and/or coupled to the ink composition 260 and the first surface of the adhesive layer 220, wherein the ink composition is deposited on and/or coupled to a portion of the first surface of the adhesive layer 220. In some embodiments, the liner 210 is a non-adhesive liner. For example, in some embodiments, the liner 210 is can be silicone coated, fluorosilicone coated, wax coated, coated with a non-stick polymer, or similarly coated with a non-stick layer. In some embodiments, the liner 210 is can be a silicone-coated paper liner. In some embodiments, the liner 210 is can be a fluorosilicone-coated paper liner.

In some embodiments, the adhesive layer 220 can be an acrylic adhesive, a rubber adhesive, or other appropriate adhesive configured to receive the ink composition, allow it to dry, and allow release and penetration of at least a portion of the composition onto and/or into the skin of a user to which it is applied.

In some embodiments, the backing 230 can be a polyurethane backing or other appropriate backing that is configured to provide, e.g., desired structure, strength, or geometry to the article. In another example, the polyurethane carrier can provide the desired structure, moisture vapor transmission properties and porosity. In some embodiments, the backing 230 can reduce and/or eliminate the stickiness of an adjacent adhesive layer. For example, the backing 230 can reduce and/or eliminate the stickiness of the adhesive layer 220. In some examples, the adhesive layer 220 can be an acrylic adhesive, a rubber adhesive, or other appropriate adhesive. In some embodiments, the backing 230 can increase the stiffness of the adhesive applicator article 200 during fabrication and/or use. In addition to or instead of increasing stiffness, the backing 230 can be transparent. In incidences when the backing 230 is transparent, a user can see the design formed by the ink composition (e.g., the ink composition 60 of FIG. 1). In some examples, when the backing 230 is transparent, the backing 230 can allow an increase in the breathability of between the skin of the user and the external environment.

In some embodiments, the adhesive applicator article 200 is configured to provide proper structure for printing, manufacturing, storage, and/or application of a composition comprising a semi-permanent colorant such as those described herein.

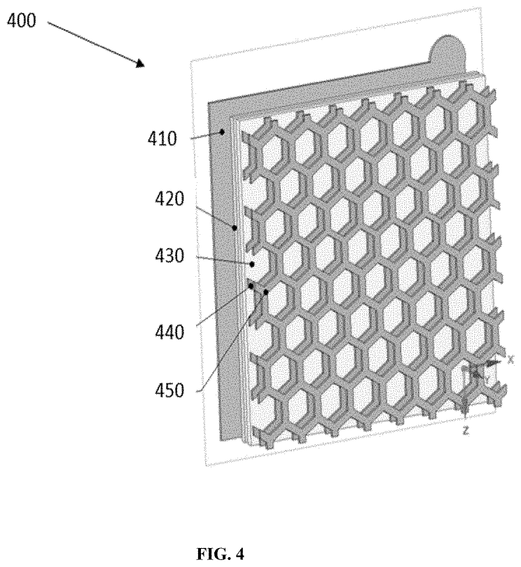

FIG. 4 illustrates an adhesive applicator article according to another embodiment of the present disclosure. As shown in FIG. 4, in some embodiments, the adhesive applicator article 400 comprises a first adhesive layer 420; a first backing 430; a second adhesive layer 440, and a second backing 450. The first adhesive layer 420 can, in some examples, be analogous to the adhesive layer 20, the first adhesive layer 120, and/or the adhesive layer 220 described in connection with FIGS. 1, 2A, 2B, and/or 3 respectively. The liner 410 can, in some examples, be analogous to the liner 10, the liner 110, and/or the liner 210 described in connection with FIGS. 1, 2A, 2B, and/or 3 respectively. The first backing 430 can, in some examples, be analogous to the adhesive backing 30, the first backing 130, and/or the backing 230 described in connection with FIGS. 1, 2A, 2B, and/or 3 respectively. The second adhesive layer 440 can, in some examples, be analogous to the adhesive layer 20, the second adhesive layer 140, and/or the adhesive layer 220 described in connection with FIGS. 1, 2A, 2B, and/or 3 respectively. The second backing 450 can, in some examples, be analogous to the adhesive backing 30, the second backing 150, and/or the backing 230 described in connection with FIGS. 1, 2A, 2B, and/or 3 respectively.

In some embodiments, the second backing is 450 strongly adhered to the second adhesive layer 440 such that its removal during application is prevented. In some embodiments, the second backing 450 can provide structure that prevents wrinkling of the adhesive layer (e.g., the first adhesive layer 420 and/or the second adhesive layer 440) during the application period. In some embodiments, the second adhesive layer 440 is a portion of the first backing 430. In some embodiments, the second backing 450 is a portion of the first backing 430. In some embodiments, the second adhesive layer 440 and the second backing 450 are a portion of the first backing 430. In some embodiments, a portion of a surface (e.g., the first portion 140a and/or the second portion 140b in connection with FIG. 2A) can include a perimeter, a portion of a perimeter, a grid, a section, and the like. In some embodiments, a portion is a honeycomb pattern, for example, as shown in FIG. 4.

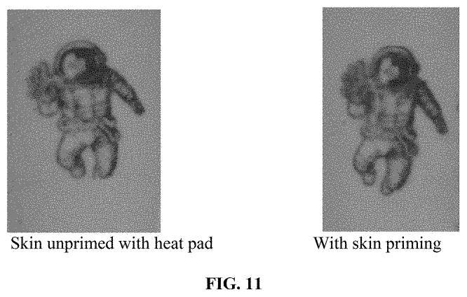

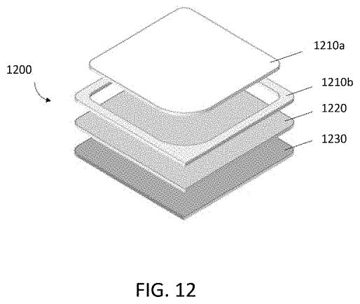

FIG. 12 is an exploded perspective view of an adhesive applicator article 1200 according to another embodiment of the present disclosure. FIG. 12 illustrates a first liner 1210a, a second liner 1210b, an adhesive layer 1220, and a backing 1230. The first liner 1210a and/or the second liner 1210b can, in some examples, be analogous to the liner 10, the liner 110, the liner 210, and/or the liner 410 described in connection with 1, 2A, 2B, 3, and 4 respectively. The adhesive layer 1220 can, in some examples, be analogous to the adhesive layer 20, the first adhesive layer 120, the second adhesive layer 140, the adhesive layer 220, the first adhesive layer 420, and/or the second adhesive layer 440 described in connection with 1, 2A, 2B, 3, and 4 respectively. The backing 1230 can, in some examples, be analogous to the adhesive backing 30, the first backing 130, the backing 230, and/or the first backing 430, described in connection with 1, 2A, 2B, 3, and 4 respectively.

In some embodiments, the first liner 1210a can be removable. For example, the first liner 1210a can be removed by a user during the application process. For instance, a user can position the adhesive applicator article 1200 and remove the first liner 1210a to expose an ink composition (e.g., the ink composition 60 of FIG. 1). In some embodiments, the second liner 1210b can be made from a material that can provide some rigidity. For example, the second liner 1210b can be a liner made from a material that can provide a structural border for the adhesive applicator article 1200. For instance, the ink composition can be a part of an adhesive layer that can be surrounded by a structural border in the form of the second liner 1210b. The second liner 1210b can be configured to provide proper structure to eliminate or reduce the risk of the adhesive including the ink composition folding in on itself, e.g., during application, and/or to improve the ease of application of the applicator article to a surface, e.g., skin surface, by a user.

The backing 1230, in some embodiments, can be a polyurethane (PU) backing or other appropriate backing that is configured to provide, e.g., desired structure, strength, or geometry to the adhesive applicator article 1200. In some examples, a PU backing 1230 can include a thickness of 1 mil. In other examples, a PU backing 1230 can include a thickness of 2.4 mil. However, embodiments are not so limited and the backing 1230 can be more or less than 1 mil or 1.4 mil. In some embodiments, the backing 1230 can reduce and/or eliminate the stickiness of an adjacent adhesive layer. For example, the backing 1230 can reduce and/or eliminate the stickiness of the adhesive layer 1220. In some embodiments, the backing 1230 can increase the stiffness of the adhesive applicator article 1200 during fabrication and/or use. An increased stiffness can reduce the risk of damage to the design formed by the ink composition. In some example embodiments, the backing 1230 can be comprised of a transparent material.

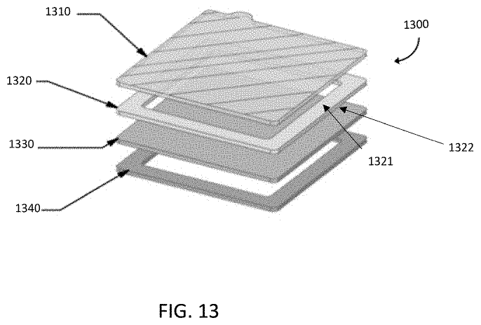

FIG. 13 is another exploded perspective view of an adhesive applicator article 1300 according to another embodiment of the present disclosure. The adhesive applicator article 1300 can be an occlusion patch article and referred to herein as occlusion patch article 1300. FIG. 13 illustrates an occlusion patch article 1300. An occlusion patch can be used with another adhesive applicator article such as the adhesive applicator article 1200 described in connection with FIG. 12. For example, the adhesive applicator article 1300 can be positioned over the adhesive applicator article 1200 to contain moisture and heat during the application process. For example, an occlusion patch can include dimensions greater than adhesive applicator article 1200 such that it can adhere to the skin arounllimd the perimeter of adhesive applicator article 1200. As shown in FIG. 13, in some embodiments the occlusion patch article 1300 comprises a liner 1310, an adhesive layer 1320; a backing 1330; and a structural layer 1340. The liner 1310 can, in some respects, be analogous to the liner 10, the liner 110, the liner 210, the liner 410, the first liner 1210a and/or the second liner 1210b described in connection with FIGS. 1, 2A, 2B, 3, 4, and 12 respectively. The adhesive layer 1320 can, in some examples, be analogous to the adhesive layer 20, the first adhesive layer 120, the second adhesive layer 140, the adhesive layer 220, the first adhesive layer 420, the second adhesive layer 440, and/or the adhesive layer 1220 described in connection with FIGS. 1, 2A, 2B, 3, 4, and 12 respectively. The backing 1330 can, in some examples, be analogous to the adhesive backing 30, the first backing 130, the second backing 150, the backing 230, the first backing 430, and/or the backing 1230 described in connection with FIGS. 1, 2A, 2B, 3, 4, and 12 respectively.

In some embodiments, the liner 1310 can be removable. For example, the liner 1310 can be removed by a user during the application process. For instance, a user can position the adhesive applicator article 1300 and remove the liner 1310 to expose an ink composition (e.g., the ink composition 60 of FIG. 1). In another example, a user can position the adhesive applicator article 1200 after removing the liner the first liner 1210a and proceed to position the occlusion patch article 1300 on top of the adhesive applicator article 1200.

In some embodiments, the adhesive layer 1320 can be a double-sided adhesive layer 1320. For example, the adhesive layer 1320 can have a first surface 1321 and a second surface 1322. The first surface 1321 can be abut or otherwise be coupled to the liner 1320 and the second surface 1322 can be opposite the first surface 1321 and abut or otherwise contact the backing 1330. In instances when the adhesive layer 1320 is double-sided, an adhesive can be disposed on both the first surface 1321 and the second surface 1322.

In some embodiments, the backing 1330 is made from a transparent material. In incidences when the backing 1330 is transparent, a user can see the design formed by the ink composition (e.g., the ink composition 60 of FIG. 1). In some examples, when the backing 1330 is transparent, the backing 1330 can allow an increase in the breathability of between the skin of the user and the external environment.

The structural layer 1340 can be a portion of the occlusion patch article 1300 that can provide a flexible frame to the ink composition design. The structural layer 1340 can be a support to protect the ink composition design and provide structure to the occlusion patch article 1300. In some embodiments the liner 1310 can be removable and/or may be a craft paper coated with silicone on the first surface 1321. In some embodiments, the double-sided adhesive layer 1320 may be a rubber synthetic adhesive such that first surface 1321 adheres to removable liner 1310 and the second surface 322 adheres to the transparent backing 1330. In some embodiments where the backing 1330 is transparent, the backing 1330 may be a polyurethane of thickness 0.5 mil to 3.0 mil. In some embodiments, layer 340 may be a roll label frame adhering to the periphery of transparent layer 330, of thickness 2-5 mil.

In some embodiments, the occlusion patch article 1300 will have larger dimensions compared to other embodiments discussed herein. For example, the occlusion patch article 1300 can have dimensions greater than the adhesive applicator article 100 described in connection with FIGS. 2A and 2B. For instance, the occlusion patch article 1300 can include dimensions greater than adhesive applicator article 100 such that it can adhere to the skin around the perimeter of adhesive applicator article 100. In some embodiments, when the applicator article 1300 adheres to the skin, the removable liner 1310 will be removed exposing adhesive layer 1320 first surface 1321 such that the first surface 1321 can adhere to the skin. The occlusion patch article 1300 can surround the adhesive layer and/or a layer comprising the ink composition.

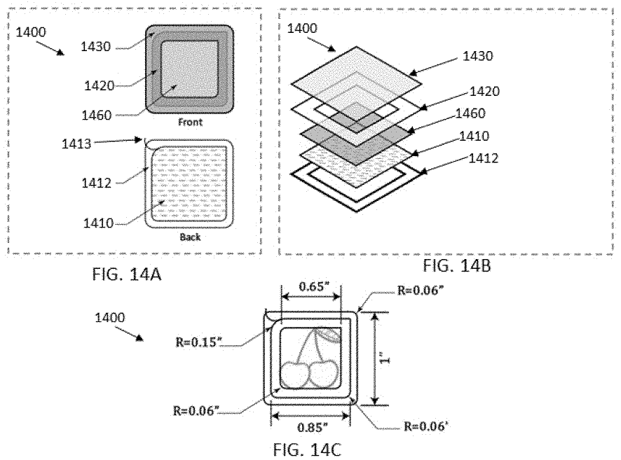

FIG. 14A, FIG. 14B, and FIG. 14C are perspective drawings of an adhesive applicator article 1400 according to an embodiment of the present disclosure. FIG. 14A is a top view of a front and a back of the adhesive applicator article 1400, FIG. 14B is an exploded view of the adhesive applicator article 1400, and FIG. 14C is an example of dimensions of the adhesive applicator article 1400. The example adhesive applicator 1400 can be an example of an occlusion patch article (e.g., the occlusion patch article 1300 described in connection with FIG. 13) with an embedded stack up 1460.

FIG. 14A illustrates a front and a back top view of the adhesive applicator article 1400. The adhesive applicator article 1400 includes a backing 1430. In some examples, the backing 1430 is a polyurethane (PU) backing. For instance, the backing 1430 can be a 1 mil PU backing. The adhesive applicator article 1400 can include an adhesive layer 1420. In some examples the adhesive layer 1420 is a double sided adhesive that can adhere to the backing 1430 and a stack up 1460. The stack up 1460 can include an adhesive layer, a double sided adhesive layer, and a 1 mil PU backing. The stack up can be positioned on top of a liner 1410. In some embodiments, the liner 1410 is a fluorosilicone (FS) liner. The liner 1410 and/or the border 1412 can be removed by a user to expose the stack up 1460 to the skin of the user. The liner can be coupled to and/or otherwise positioned with a border 1412. The border 1412 can provide a guide for the user as well as structure to the adhesive applicator article 1400. The adhesive applicator article 1400 can further include a pull-tab 1430 to facilitate the removal of the border 1412. For example, the pull-tab 1413 can facilitate the removal of the border 1410. After the removal of the border 1410 using the pull-tab 1413, the user can remove the liner 1410. The liner 1410 can facilitate the preservation and/or the shelf life of the ink composition.

FIG. 14B illustrates an exploded view of the adhesive applicator article 1400. The exploded view illustrates an example of the different elements of the adhesive applicator article 1400. In this instance, the adhesive applicator article 1400 includes a backing (e.g., a 1 mil PU backing) to couple to an adhesive layer 1420 (e.g., a double-sided adhesive layer 1420), to couple to a stack up 1460. The stack up 1460 can be coupled to a liner 1410 (e.g., a FS liner) which can be removed to expose the stack up 1460 to the skin of a user. The border 1412 can provide structure to the adhesive applicator article 1400 and facilitate the position and adherence to the skin of a user and/or the handling and position to the skin of the user.

FIG. 14C includes an example design with example measurements. While specific numbers are illustrated in FIG. 14, embodiments are not so limited. Multiple different dimensions have been considered and may be more or less than the dimensions illustrated in FIG. 14. As mentioned herein, an occlusion patch article can, in some embodiments, have larger dimensions compared to other embodiments discussed herein. For example, an occlusion patch article (e.g., the adhesive applicator article 1400) can have dimensions greater than the adhesive applicator article 1200 described in connection with FIG. 12.

The adhesive applicator article 1400 can be a combination of the adhesive applicator article 1200 and the adhesive applicator article 1300. Combining these two adhesive applicator articles can reduce waste and performance issues and user error involved with positioning article 1200 over article 1300. Combining the two articles can reduce the potential for wrinkles and user error.

The embodiment illustrated in FIG. 14C illustrates border (e.g., the border 1412 of FIGS. 14A and 14B) with a dimension of 1.0 Inch (in), an adhesive layer (e.g., the adhesive layer 1420 of FIGS. 14A and 14B) including a dimension of 0.85 in, and a stack up (e.g., the stack up 1460 of FIGS. 14A and 14B) including a dimension of 0.65 in. The adhesive layer and/or the stack up can include a design comprising an ink composition (e.g., the ink composition 60 as described above in connection with FIG. 1). The border can have a radius of curvature (R) R=0.15 in, adhesive layer can have a radius of curvature of R=0.06 in, and the stack up can have a radius of curvature of R=0.06 in.

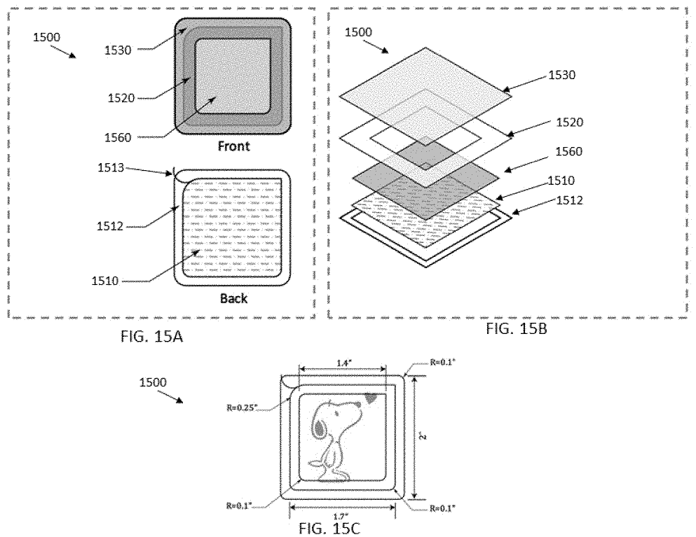

FIG. 15A, FIG. 15B, and FIG. 15C are perspective drawings of an adhesive applicator article 1500 according to an embodiment of the present disclosure. FIG. 15A is a top view of a front and a back of the adhesive applicator article 1500, FIG. 15B is an exploded view of the adhesive applicator article 1500, and FIG. 15C is an example of dimensions of the adhesive applicator article 1500. The example adhesive applicator 1500 can be an example of an occlusion patch article (e.g., the occlusion patch article 1300 described in connection with FIG. 13) with an embedded stack up 1560.

FIG. 15A illustrates a front and a back top view of the adhesive applicator article 1500. The adhesive applicator article 1500 includes a backing 1530. In some examples, the backing 1530 is a PU backing. For instance, the backing 1530 can be a 1 mil PU backing. The adhesive applicator article 1500 can include an adhesive layer 1520. In some examples the adhesive layer 1520 is a double sided adhesive that can adhere to the backing 1530 and a stack up 1560. The stack up 1560 can include an adhesive layer, a double sided adhesive layer, and a 1 mil PU backing. The stack up can be positioned on top of a liner 1510. In some embodiments, the liner 1510 is a FS liner. The liner 1510 and/or the border 1512 can be removed by a user to expose the stack up 1560 to the skin of the user. The liner can be coupled to and/or otherwise positioned with a border 1512. The border 1512 can provide a guide for the user as well as structure to the adhesive applicator article 1500. The adhesive applicator article 1500 can further include a pull-tab 1530 to facilitate the removal of the border 1512. For example, the pull-tab 1513 can facilitate the removal of the border 1510. After the removal of the border 1510 using the pull-tab 1513, the user can remove the liner 1510. The liner 1510 can facilitate the preservation and/or the shelf life of the ink composition.

FIG. 15B illustrates an exploded view of the adhesive applicator article 1500. The exploded view illustrates an example of the different elements of the adhesive applicator article 1500. In this instance, the adhesive applicator article 1500 includes a backing (e.g., a 1 mil PU backing) to couple to an adhesive layer 1520 (e.g., a double-sided adhesive layer 1520), to couple to a stack up 1560. The stack up 1560 can be coupled to a liner 1510 (e.g., a FS liner) which can be removed to expose the stack up 1560 to the skin of a user. The border 1512 can provide structure to the adhesive applicator article 1500 and facilitate the position and adherence to the skin of a user and/or the handling and position to the skin of the user.

FIG. 15C includes an example design with example measurements. While specific numbers are illustrated in FIG. 15, embodiments are not so limited. Multiple different dimensions have been considered and may be more or less than the dimensions illustrated in FIG. 15. As mentioned herein, an occlusion patch article can, in some embodiments, have larger dimensions compared to other embodiments discussed herein. For example, an occlusion patch article (e.g., the adhesive applicator article 1500) can have dimensions greater than the adhesive applicator article 1200 described in connection with FIG. 12.

The adhesive applicator article 1500 can be a combination of the adhesive applicator article 1200 and the adhesive applicator article 1300. Combining these two adhesive applicator articles can reduce waste and performance issues and user error involved with positioning article 1200 over article 1300. Combining the two articles can reduce the potential for wrinkles and user error.

The embodiment illustrated in FIG. 15C illustrates border (e.g., the border 1512 of FIGS. 15A and 15B) with a dimension of 2.0 Inches (in), an adhesive layer (e.g., the adhesive layer 1520 of FIGS. 15A and 15B) including a dimension of 1.7 in, and a stack up (e.g., the stack up 1560 of FIGS. 15A and 15B) including a dimension of 1.4 in. The adhesive layer and/or the stack up can include a design comprising an ink composition (e.g., the ink composition 60 as described above in connection with FIG. 1). The border can have a radius of curvature (R) R=0.25 in, adhesive layer can have a radius of curvature of R=0.1 in, and the stack up can have a radius of curvature of R=0.1 in.

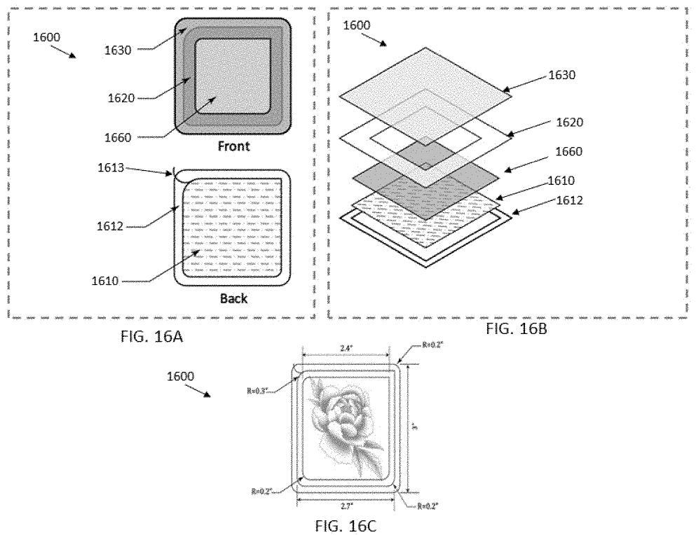

FIG. 16A, FIG. 16B, and FIG. 16C are perspective drawings of an adhesive applicator article 1600 according to an embodiment of the present disclosure. FIG. 16A is a top view of a front and a back of the adhesive applicator article 1600, FIG. 16B is an exploded view of the adhesive applicator article 1600, and FIG. 16C is an example of dimensions of the adhesive applicator article 1600. The example adhesive applicator 1600 can be an example of an occlusion patch article (e.g., the occlusion patch article 1300 described in connection with FIG. 13) with an embedded stack up 1660.

FIG. 16A illustrates a front and a back top view of the adhesive applicator article 1600. The adhesive applicator article 1600 includes a backing 1630. In some examples, the backing 1630 is a PU backing. For instance, the backing 1630 can be a 1 mil PU backing. The adhesive applicator article 1600 can include an adhesive layer 1620. In some examples the adhesive layer 1620 is a double sided adhesive that can adhere to the backing 1630 and a stack up 1660. The stack up 1660 can include an adhesive layer, a double sided adhesive layer, and a 1 mil PU backing. The stack up can be positioned on top of a liner 1610. In some embodiments, the liner 1610 is a FS liner. The liner 1610 and/or the border 1612 can be removed by a user to expose the stack up 1660 to the skin of the user. The liner can be coupled to and/or otherwise positioned with a border 1612. The border 1612 can provide a guide for the user as well as structure to the adhesive applicator article 1600. The adhesive applicator article 1600 can further include a pull-tab 1630 to facilitate the removal of the border 1612. For example, the pull-tab 1613 can facilitate the removal of the border 1610. After the removal of the border 1610 using the pull-tab 1613, the user can remove the liner 1610. The liner 1610 can facilitate the preservation and/or the shelf life of the ink composition.

FIG. 16B illustrates an exploded view of the adhesive applicator article 1600. The exploded view illustrates an example of the different elements of the adhesive applicator article 1600. In this instance, the adhesive applicator article 1600 includes a backing (e.g., a 1 mil PU backing) to couple to an adhesive layer 1620 (e.g., a double-sided adhesive layer 1620), to couple to a stack up 1660. The stack up 1660 can be coupled to a liner 1610 (e.g., a FS liner) which can be removed to expose the stack up 1660 to the skin of a user. The border 1612 can provide structure to the adhesive applicator article 1600 and facilitate the position and adherence to the skin of a user and/or the handling and position to the skin of the user.

FIG. 16C includes an example design with example measurements. While specific numbers are illustrated in FIG. 16, embodiments are not so limited. Multiple different dimensions have been considered and may be more or less than the dimensions illustrated in FIG. 16. As mentioned herein, an occlusion patch article can, in some embodiments, have larger dimensions compared to other embodiments discussed herein. For example, an occlusion patch article (e.g., the adhesive applicator article 1600) can have dimensions greater than the adhesive applicator article 1200 described in connection with FIG. 12.

The adhesive applicator article 1500 can be a combination of the adhesive applicator article 1200 and the adhesive applicator article 1300. Combining these two adhesive applicator articles can reduce waste and performance issues and user error involved with positioning article 1200 over article 1300. Combining the two articles can reduce the potential for wrinkles and user error.

The embodiment illustrated in FIG. 16C illustrates border (e.g., the border 1612 of FIGS. 16A and 16B) with a dimension of 3.0 Inches (in), an adhesive layer (e.g., the adhesive layer 1620 of FIGS. 16A and 16B) including a dimension of 2.7 in, and a stack up (e.g., the stack up 1660 of FIGS. 16A and 16B) including a dimension of 2.4 in. The adhesive layer and/or the stack up can include a design comprising an ink composition (e.g., the ink composition 60 as described above in connection with FIG. 1). The border can have a radius of curvature (R) R=0.3 in, adhesive layer can have a radius of curvature of R=0.2 in, and the stack up can have a radius of curvature of R=0.2 in.

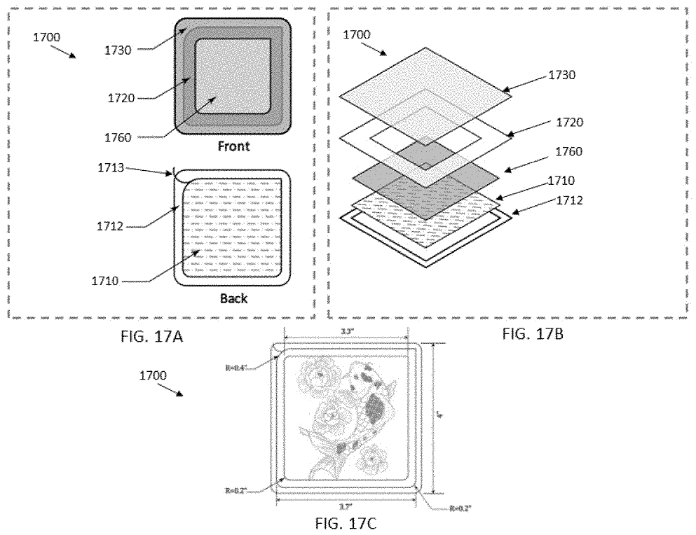

FIG. 17A, FIG. 17B, and FIG. 17C are perspective drawings of an adhesive applicator article 1700 according to an embodiment of the present disclosure. FIG. 17A is a top view of a front and a back of the adhesive applicator article 1700, FIG. 17B is an exploded view of the adhesive applicator article 1700, and FIG. 17C is an example of dimensions of the adhesive applicator article 1700. The example adhesive applicator 1700 can be an example of an occlusion patch article (e.g., the occlusion patch article 1300 described in connection with FIG. 13) with an embedded stack up 1760.

FIG. 17A illustrates a front and a back top view of the adhesive applicator article 1700. The adhesive applicator article 1700 includes a backing 1730. In some examples, the backing 1730 is a PU backing. For instance, the backing 1730 can be a 1 mil PU backing. The adhesive applicator article 1700 can include an adhesive layer 1720. In some examples the adhesive layer 1720 is a double sided adhesive that can adhere to the backing 1730 and a stack up 1760. The stack up 1760 can include an adhesive layer, a double sided adhesive layer, and a 1 mil PU backing. The stack up can be positioned on top of a liner 1710. In some embodiments, the liner 1710 is a FS liner. The liner 1710 and/or the border 1712 can be removed by a user to expose the stack up 1760 to the skin of the user. The liner can be coupled to and/or otherwise positioned with a border 1712. The border 1712 can provide a guide for the user as well as structure to the adhesive applicator article 1700. The adhesive applicator article 1700 can further include a pull-tab 1730 to facilitate the removal of the border 1712. For example, the pull-tab 1713 can facilitate the removal of the border 1710. After the removal of the border 1710 using the pull-tab 1713, the user can remove the liner 1710. The liner 1710 can facilitate the preservation and/or the shelf life of the ink composition.

FIG. 17B illustrates an exploded view of the adhesive applicator article 1700. The exploded view illustrates an example of the different elements of the adhesive applicator article 1700. In this instance, the adhesive applicator article 1700 includes a backing (e.g., a 1 mil PU backing) to couple to an adhesive layer 1720 (e.g., a double-sided adhesive layer 1720), to couple to a stack up 1760. The stack up 1760 can be coupled to a liner 1710 (e.g., a FS liner) which can be removed to expose the stack up 1760 to the skin of a user. The border 1712 can provide structure to the adhesive applicator article 1700 and facilitate the position and adherence to the skin of a user and/or the handling and position to the skin of the user.

FIG. 17C includes an example design with example measurements. While specific numbers are illustrated in FIG. 17, embodiments are not so limited. Multiple different dimensions have been considered and may be more or less than the dimensions illustrated in FIG. 17. As mentioned herein, an occlusion patch article can, in some embodiments, have larger dimensions compared to other embodiments discussed herein. For example, an occlusion patch article (e.g., the adhesive applicator article 1700) can have dimensions greater than the adhesive applicator article 1200 described in connection with FIG. 12.

The adhesive applicator article 1700 can be a combination of the adhesive applicator article 1200 and the adhesive applicator article 1300. Combining these two adhesive applicator articles can reduce waste and performance issues and user error involved with positioning article 1200 over article 1300. Combining the two articles can reduce the potential for wrinkles and user error.

The embodiment illustrated in FIG. 17C illustrates border (e.g., the border 1712 of FIGS. 17A and 17B) with a dimension of 4.0 Inches (in), an adhesive layer (e.g., the adhesive layer 1720 of FIGS. 17A and 17B) including a dimension of 3.7 in, and a stack up (e.g., the stack up 1760 of FIGS. 17A and 17B) including a dimension of 3.3 in. The adhesive layer and/or the stack up can include a design comprising an ink composition (e.g., the ink composition 60 as described above in connection with FIG. 1). The border can have a radius of curvature (R) R=0.4 in, adhesive layer can have a radius of curvature of R=0.2 in, and the stack up can have a radius of curvature of R=0.2 in.

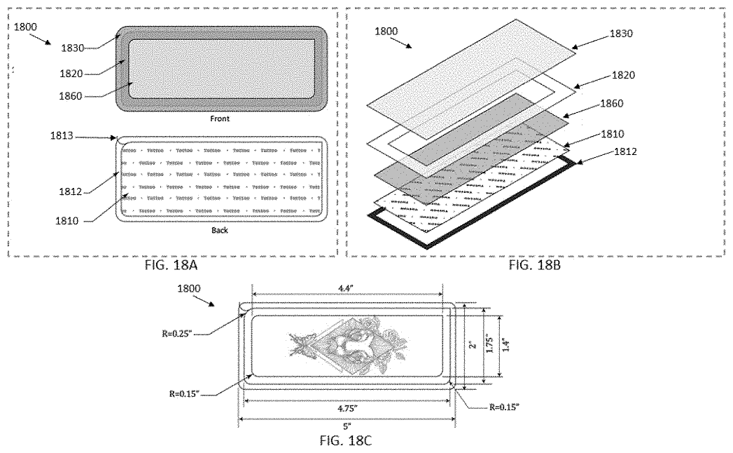

FIG. 18A, FIG. 18B, and FIG. 18C are perspective drawings of an adhesive applicator article 1800 according to an embodiment of the present disclosure. FIG. 18A is a top view of a front and a back of the adhesive applicator article 1800, FIG. 18B is an exploded view of the adhesive applicator article 1800, and FIG. 18C is an example of dimensions of the adhesive applicator article 1800. The example adhesive applicator 1800 can be an example of an occlusion patch article (e.g., the occlusion patch article 1300 described in connection with FIG. 13) with an embedded stack up 1860.

FIG. 18A illustrates a front and a back top view of the adhesive applicator article 1800. The adhesive applicator article 1800 includes a backing 1830. In some examples, the backing 1830 is a PU backing. For instance, the backing 1830 can be a 1 mil PU backing. The adhesive applicator article 1800 can include an adhesive layer 1820. In some examples the adhesive layer 1820 is a double sided adhesive that can adhere to the backing 1830 and a stack up 1860. The stack up 1860 can include an adhesive layer, a double sided adhesive layer, and a 1 mil PU backing. The stack up can be positioned on top of a liner 1810. In some embodiments, the liner 1810 is a FS liner. The liner 1810 and/or the border 1812 can be removed by a user to expose the stack up 1860 to the skin of the user. The liner can be coupled to and/or otherwise positioned with a border 1812. The border 1812 can provide a guide for the user as well as structure to the adhesive applicator article 1800. The adhesive applicator article 1800 can further include a pull-tab 1830 to facilitate the removal of the liner 1820. For example, the pull-tab 1813 can facilitate the removal of the border 1810. After the removal of the border 1810 using the pull-tab 1813, the user can remove the liner 1810. The liner 1810 can facilitate the preservation and/or the shelf life of the ink composition.

FIG. 18B illustrates an exploded view of the adhesive applicator article 1800. The exploded view illustrates an example of the different elements of the adhesive applicator article 1800. In this instance, the adhesive applicator article 1800 includes a backing (e.g., a 1 mil PU backing) to couple to an adhesive layer 1820 (e.g., a double-sided adhesive layer 1820), to couple to a stack up 1860. The stack up 1860 can be coupled to a liner 1810 (e.g., a FS liner) which can be removed to expose the stack up 1860 to the skin of a user. The border 1812 can provide structure to the adhesive applicator article 1800 and facilitate the position and adherence to the skin of a user and/or the handling and position to the skin of the user.

FIG. 18C includes an example design with example measurements. While specific numbers are illustrated in FIG. 18, embodiments are not so limited. Multiple different dimensions have been considered and may be more or less than the dimensions illustrated in FIG. 18. As mentioned herein, an occlusion patch article can, in some embodiments, have larger dimensions compared to other embodiments discussed herein. For example, an occlusion patch article (e.g., the adhesive applicator article 1800) can have dimensions greater than the adhesive applicator article 1200 described in connection with FIG. 12.

The adhesive applicator article 1800 can be a combination of the adhesive applicator article 1200 and the adhesive applicator article 1300. Combining these two adhesive applicator articles can reduce waste and performance issues and user error involved with positioning article 1200 over article 1300. Combining the two articles can reduce the potential for wrinkles and user error.

The embodiment illustrated in FIG. 18C illustrates border (e.g., the border 1812 of FIGS. 18A and 18B) with a dimension of length 5 Inches (in) and width 2.0 in, an adhesive layer (e.g., the adhesive layer 1820 of FIGS. 18A and 18B) including a dimension of length 4.75 in and width 1.75 in, and a stack up (e.g., the stack up 1860 of FIGS. 18A and 18B) including a dimension of length 4.4 in and a width of 1.4 in. The adhesive layer and/or the stack up can include a design comprising an ink composition (e.g., the ink composition 60 as described above in connection with FIG. 1). The border can have a radius of curvature (R) R=0.25 in, adhesive layer can have a radius of curvature of R=0.15 in, and the stack up can have a radius of curvature of R=0.15 in.

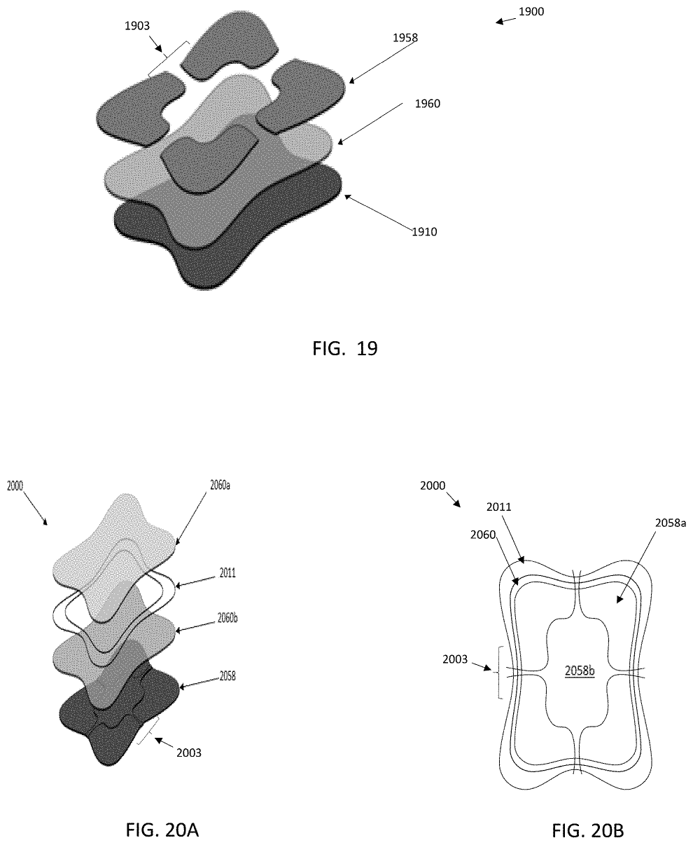

FIG. 19 is another example of an adhesive applicator article 1900 according to another embodiment of the present disclosure. The adhesive applicator article 1900 can include an aperture 1903, one or more portions of a backing 1958, a stack up 1960, and a liner 1910. While one aperture 1903 is identified in FIG. 19, there can be more or less than one aperture 1903. The stack up 1960 can be analogous to the stack up 1460, 1560, 1660, 1760, and 1860 described in connection with FIGS. 14, 15, 16, 17, and 18 respectively. The liner can be an FS liner which can be selectively removable by a user to expose the stack up 1960 to the skin of the user. The backing 1958 can be a removable backing. In some examples the backing 1958 can be a removable paper backing that can be removed by a user during the application of the adhesive applicator article 1900. FIG. 19 labels a portion of the backing 1958, however, as illustrated in FIG. 19, there are four portions of the backing 1958. While four portions of the backing 1958 are illustrated in the example of the adhesive applicator article 1900, embodiments are not so limited and there may be more or less than four portions of backing 1958.

The adhesive applicator article 1900 can include one or more apertures 1903 which can facilitate the stiffness of the adhesive applicator article 1900 during the application of the adhesive applicator article 1900 onto a body part of a user. For example, the adhesive applicator article 1900 can be a design for large designs (e.g., oversized tattoo designs). As illustrated in FIG. 19 the shape of the adhesive applicator article 1900 can be curved to provide a butterfly shape. The butterfly shape of the adhesive applicator article 1900 can adapt to the contours of the body. The butterfly shape in combination with the aperture(s) 1903 can prevent and/or reduce lifting and delamination of the adhesive applicator article 1900 once an oversized tattoo applicator is applied on any convex body surfaces (e.g., biceps, forearms, thighs, shoulder, etc.). The incorporation of a structural layer such as the paper backing 1958 on top of the adhesive applicator article 1900 can provide further structural support to prevent wrinkles and/or facilitate further wrinkle free application. A user can remove the FS liner 1910 to expose the adhesive layer included in (e.g., in contact with) the stack up 1960 to apply the design to the skin. The user may smooth out any wrinkles by applying pressure to the paper backing 1958 and/or by smoothing out the paper backing 1958. When the user is satisfied with the application, they can remove the one or more superficial paper backing 1958 portions.

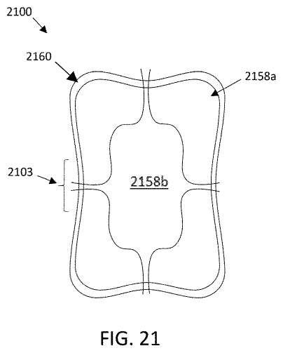

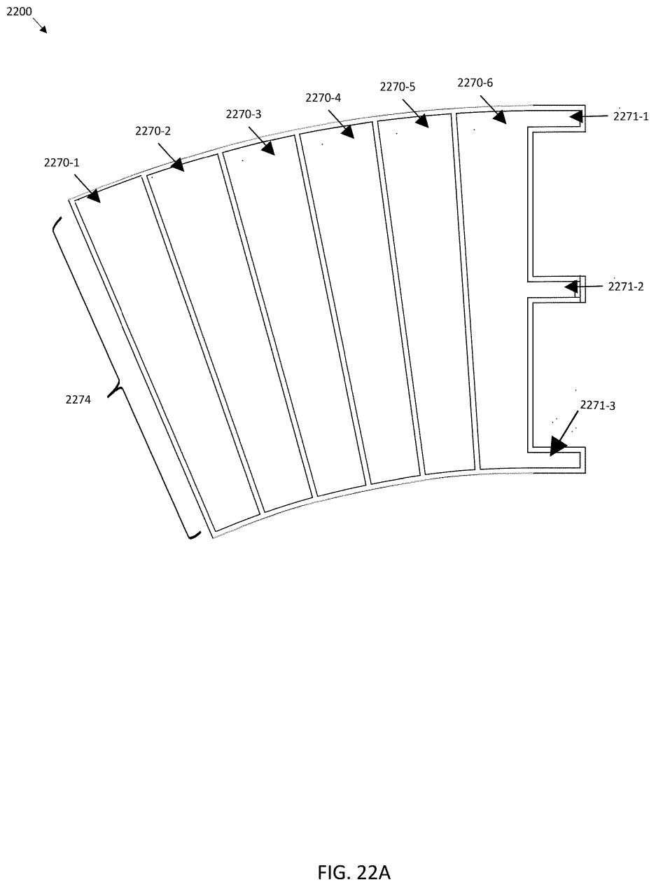

In another example embodiment, structural support can be further provided to the adhesive applicator article 1900 by cutting through a FS liner on the adhesive side of the stack up and/or providing a border. These embodiments are discussed in connection with FIGS. 20A, 20B, and 21.

FIGS. 20A and 20B illustrate perspective views of an example adhesive applicator article 2000 according to another embodiment of the present disclosure. FIG. 20A is an exploded view of the layers of the example adhesive applicator article 2000 described in connection with FIG. 20B. FIG. 20B is a bottom view of the example adhesive applicator article 2000.

FIG. 20A shows an exploded view of layers of the adhesive applicator article 2000. The adhesive applicator article 2000 includes one or more apertures 2003, a liner 2060a (e.g., a lmil PU liner), an adhesive layer (e.g., a double sided adhesive layer) 2011, a stack up 2060b, and an FS liner 2058.

While one aperture 2003 is identified in FIGS. 20A and 20B, there can be more or less than one aperture 2003. The stack up 2060 of FIGS. 20A and 20B can be analogous to the stack up 1460, 1560, 1660, 1760, and 1860 described in connection with FIGS. 14, 15, 16, 17, and 18 respectively. The adhesive layer 2011 of FIGS. 20A and 20B can provide structure and rigidity to the adhesive applicator article 2000.

The FS liner 2058 can be removed in portions as described in connection with FIG. 20A and the adhesive layer 2011 can provide support to the adhesive applicator article 2000 during application and/or during the removal of the FS liner 2058. FIGS. 20A and 20B label a portion of the FS liner 2058, however, as illustrated in FIGS. 20A and 20B, there are four portions of the backing 2058. While four portions of the backing 2058 are illustrated in the example of the adhesive applicator article 2000, embodiments are not so limited and there may be more or less than four portions of backing 2058.

FIG. 20B illustrates layer 2060 which is an overlapped view of liner 2060a combined with stack up 2060b described in connection with FIG. 20A. FIG. 20B further illustrates the adhesive layer 2011 which can provide support during application. FIG. 20B further illustrates FS liner 2058 in two sections 2058a and 2058b. A user can remove the center portion 2058b to expose the design imprinted on the stack up 2060. The user can position the design onto the skin and smooth out wrinkles. When the user is satisfied with the placement, the user can remove the four portions of the FS liner 2058a. During this application process, the double sided adhesive layer 2011 can provide a strong structural border. The example adhesive applicator article 2000 can include extending one or more of the apertures 2003 through the FS liner 2058 such that the FS liner 2058 can act as a structural border. The butterfly shape of the adhesive applicator article 2000 can adapt to the contours of the body

The butterfly shape in combination with the aperture(s) 2003 extending through the FS liner 2058 can prevent and/or reduce lifting and delamination of the adhesive applicator article 2000 once an oversized tattoo applicator is applied on any convex body surfaces (e.g., biceps, forearms, thighs, shoulder, etc.). The incorporation of a structural layer 2011 can provide further structural support to prevent wrinkles and/or facilitate further wrinkle free application.

FIG. 21 is another example of an adhesive applicator article 2100 according to another embodiment of the present disclosure. FIG. 21 is a bottom up view that is similar to FIGS. 20A and 20B except it is without the adhesive layer to provide a structural border. Adhesive applicator article 2100 includes an overlapped combination of a stack up and an FS liner 2160. The adhesive applicator article 2100 can include one or more apertures 2103, one or more portions of a backing 2158a and a center portion of the backing 2058b. While one aperture 2103 is identified in FIG. 21, there can be more or less than one aperture 2103. The stack up included in 2160 can be analogous to the stack up 1460, 1560, 1660, 1760, and 1860 described in connection with FIGS. 14, 15, 16, 17, and 18 respectively.

The backing 2158 can be a removable backing. In some examples the backing 2158 can be a removable paper backing and/or and FS liner that can be removed by a user during the application of the adhesive applicator article 2100. FIG. 21 labels a portion of the backing 2158, however, as illustrated in FIG. 21, there are four portions of the backing 2158. While four portions of the backing 2158 are illustrated in the example of the adhesive applicator article 2100, embodiments are not so limited and there may be more or less than four portions of backing 2158. The example adhesive applicator article 2100 can include extending one or more of the apertures 2103 through the FS liner (not illustrated) on the adhesive side of the stack up 2160 in order to provide structural support to the adhesive applicator article 2100.

The extension of the one or more of the apertures 2103 through the FS liner on the adhesive side of the stack up included in 2160 can provide support and better adhesion by removing the backing 2058a and 2158b into smaller parts of the article 2100 from lifting off of the users skin when applied to convex surfaces. The butterfly shape of the adhesive applicator article 2100 can adapt to the contours of the body. The butterfly shape in combination with the aperture(s) 2103 extending through the FS liner can prevent and/or reduce lifting and delamination of the adhesive applicator article 2100 once an oversized adhesive applicator is applied on any convex body surfaces (e.g., biceps, forearms, thighs, shoulder, etc.). The incorporation of a structural layer such as the paper backing 2158 on top of the adhesive applicator article 2100 can provide further structural support to prevent wrinkles and/or facilitate further wrinkle free application.

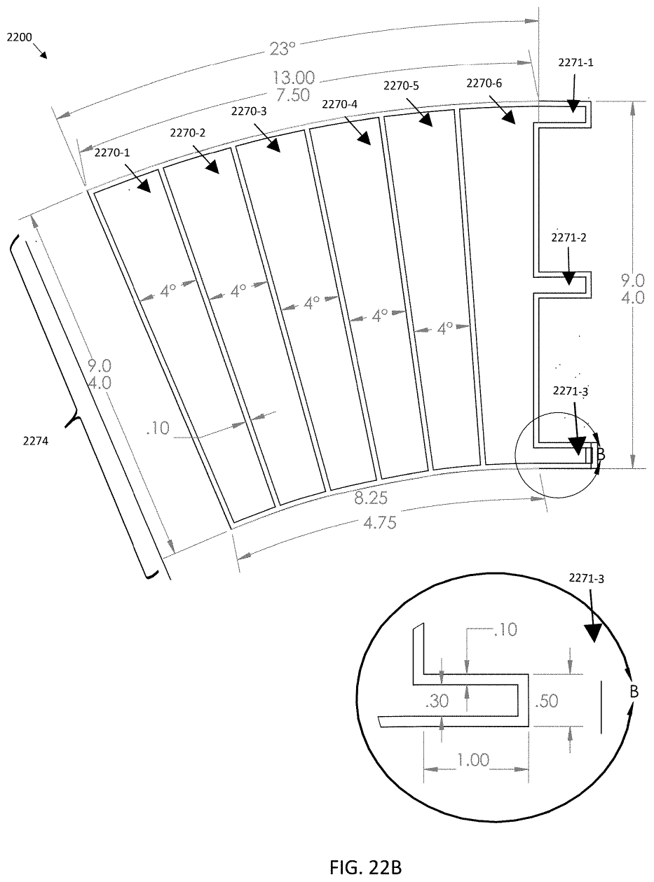

FIGS. 22A and 22B illustrate another example of an adhesive applicator article 2200 according to another embodiment of the present disclosure. The example of adhesive applicator article 2200 is an example of an adhesive applicator designed to provide the placement of a design to a convex portion of a user. For example, the adhesive applicator article 2200 can be applied to such parts of a user's body as a forearm, a thigh, a shoulder, a bicep, etc.

Both FIG. 22A and FIG. 22B include a plurality of panels that can include a design comprising an ink composition (e.g., the ink composition 60 described in connection with FIG. 1). The panels 2270-1, 2270-2, 2270-3, 2270-4, 2270-5, and 2270-6 can be collectively referred to herein as the panels 2270. While six panels 2270 are illustrated in FIG. 22A and FIG. 22B, embodiments are not so limited and embodiments can include more or less than six panels 2270.

Although not specifically labeled as to not obscure examples of the disclosure, the adhesive applicator article 2200 can include any of the elements described in connection with FIGS. 1-21. For example, the adhesive applicator article 2200 can include adhesive layers, backings, liners, ink compositions, and designs, among other elements as described herein. The adhesive applicator article 2200 can include an external layer. The external layer may be comprised of can include adhesive layers, backings, liners, etc. The external layer can be opposite the portion of the adhesive applicator article 2200 that contacts the skin of the user.

As mentioned, the adhesive applicator article 2200 can be used for a convex portion of a user's body such as a forearm. The adhesive applicator article 2200 illustrated by FIGS. 22A and 22B can include one or more tabs 2271-1, 2271-2, and 2271-3 which can be referred to herein as tabs 2271. While three tabs 2271 are illustrated in FIG. 22A and FIG. 22B, embodiments are not so limited and embodiments can include more or less than three tabs 2271. The tabs 2271 are included to prevent and/or reduce delamination and to give the user a direction of application. For example, a user can apply the adhesive applicator article 2200 to their forearm and, using the tabs 2271, wrap the adhesive applicator article 2200 such that the tabs 2271 contact the portion 2274. The portion 2274 can contacted by the tabs 2271 can include the external layer. The tabs 2271 can adhere to the outer layer of the adhesive applicator article 2200 after wrapping around the forearm. The tabs 2271 contacting the portion 2274 can keep the adhesive applicator article 2200 on the skin of the user even during movement of the user.

FIG. 22B illustrates example dimensions (all dimensions are illustrated in inches). For example, the top of the tabs 2270 span from 7.5 in to 13.0 in (not including the tabs 2271) at an arc of 23 degrees. The bottom of the tabs 2270 span 8.25 in including the tabs 2271 and 4.75 in not including the tabs 2271. The height of the adhesive applicator article 2200 is can be (for example) between 9.0 and 4.0 in. Each panel 2270 includes a 4 degree arc with a 0.10 in space that can act as a border. FIG. 2B includes a zoomed in perspective of the tab 2271-3. The tab 2271-3 has the dimension of a 0.10 in border where each tab includes a width of 0.50 in and a length of 1.0 in with a 0.30 in space between the borders of each tab 2271. While specific numbers are listed and illustrated herein, embodiments are not so limited, and different sizes and configurations of adhesive applicator article 2200 have been contemplated to accommodate users of differing sizes.

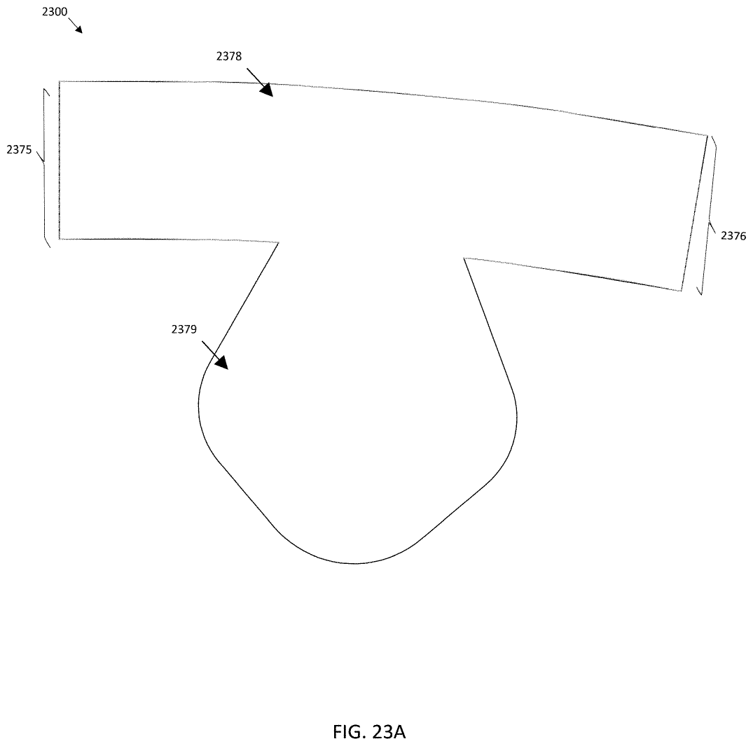

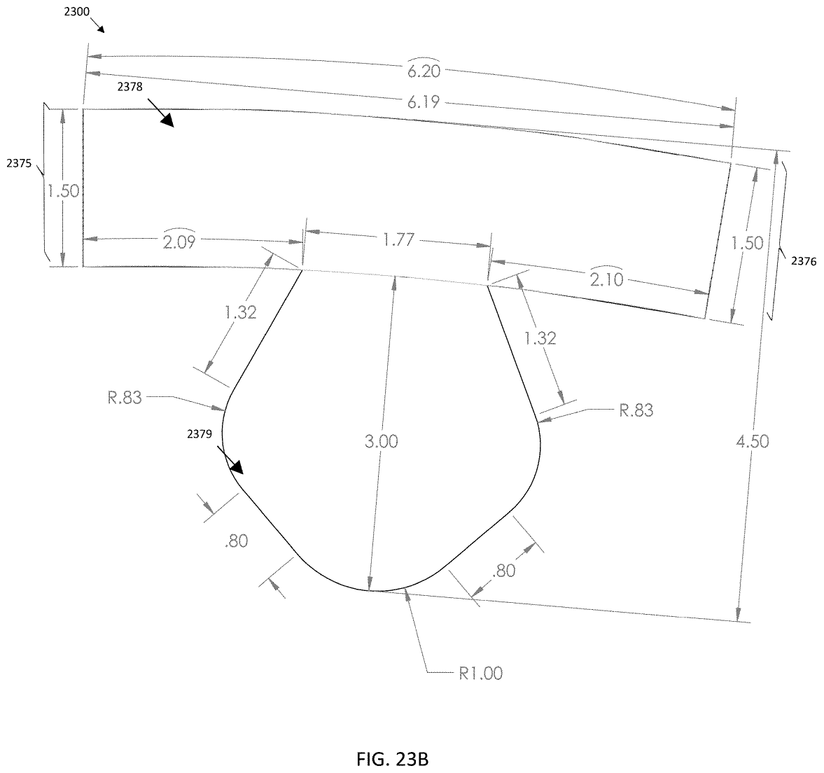

FIGS. 23A and 23B illustrate another example of an adhesive applicator article 2300 according to another embodiment of the present disclosure. The example of adhesive applicator article 2300 is an example of an adhesive applicator designed to provide the placement of a design to a convex and/or moving portion of a user. For example, the adhesive applicator article 2300 can be applied to such parts of a user's hand, foot, wrist and/or ankle, etc.

Although not specifically labeled as to not obscure examples of the disclosure, the adhesive applicator article 2300 can include any of the elements described in connection with FIGS. 1-22. For example, the adhesive applicator article 2300 can include adhesive layers, backings, liners, ink compositions, and designs, among other elements as described herein. The adhesive applicator article 2300 can include an external layer. The external layer may be comprised of can include adhesive layers, backings, liners, etc. The external layer can be opposite the portion of the adhesive applicator article 2300 that contacts the skin of the user.

Both FIGS. 23A and 23B include a first portion 2378 having a left side 2375 (e.g., a first side), a right side 2376 (e.g., a second side), and a second portion 2379. The first portion 2378 and/or the second portion 2379 can include an ink composition (e.g., the ink composition 60 described in connection with FIG. 1). The left side 2375 and the right side 2376 are included to prevent and/or reduce delamination and to give the user a direction of application. For example, a user can apply the adhesive applicator article 2300 to their hand and/or wrist and, using the left side 2375 and the right side 2376, wrap the adhesive applicator article 2300 such that left side 2375 contacts the right side 2376. The left side 2375 and the right side 2376 can adhere to the outer layer of each other after wrapping around the wrist. The left side 2375 and the right side 2376 can keep the adhesive applicator article 2300 on the skin of the user even during movement of the user. Using these methods the second portion 2379 can be positioned to provide a design to the top of a user's hand and the first portion 2378 can be positioned to provide a design to the wrist of a user.

FIG. 23B illustrates example dimensions (all dimensions are illustrated in inches). For example, from the left side 2375 to the right side 2376 the first portion 2378 can be 6.19 in and 6.20 in including the curved portion. The width of the left side 2375 and the right side 2376 can be 1.50 in and the distance from the left side 2375 and the right side 2376 to the second portion 2376 can be 2.10 in. The flat portion of the second portion 2379 can be 1.32 in while the curved portions have an R=0.83 in. The end of the second portion 2379 can include an R=1.00 in with flat portions of 0.80 in between. The distance from the top of the first portion 2378 to the bottom of the second portion 2379 can be 4.50 in. While specific numbers are listed and illustrated herein, embodiments are not so limited, and different sizes and configurations of adhesive applicator article 2300 have been contemplated to accommodate users of differing sizes.

Also provided herein are skin priming compositions comprising one or more volatile solvents; one or more amines; and one or more humectants.

In some embodiments, the one or more volatile solvents are present in the skin priming composition in an amount of about 60% to about 98% w/w of the composition. For example, about 60% to about 70%, about 60% to about 75%, about 60% to about 80%, about 60% to about 85%, about 60% to about 90%, about 60% to about 95%, about 95% to about 98%, about 90% to about 98%, about 85% to about 98%, about 80% to about 98%, about 75% to about 98%, or about 70% to about 98% w/w of the composition. In some embodiments, the one or more volatile solvents are present in the skin priming composition in an amount of about 80% to about 90%, about 85% to about 95%, about 85% to about 90%, or about 90% to about 95% w/w of the composition. In some embodiments, the one or more volatile solvents are present in the skin priming composition in an amount of about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, or about 98% w/w of the composition.

In some embodiments, non-limiting examples of the one or more volatile solvents in the skin priming composition include: water, methanol, ethanol, and isopropanol.

An “amine” as referred to herein describes a compound having a functional group that contains a basic nitrogen atom with a lone pair. For example, an amine includes a derivative of ammonia (NH₃) wherein one or more of the hydrogen atoms have been replaced with a substituent group. Non-limiting examples of an amine include an amino acid, panthenol, triethanolamine, and other compounds with primary, secondary or tertiary amines. As used herein, the term “amino acid” refers to naturally and non-naturally occurring L- and D-amino acids, peptidomimetic amino acids, and non-standard amino acids that are not made by a standard machinery or are only found in proteins after post-translational modification or as metabolic intermediates. Exemplary amino acids include, but are not limited to, arginine, alanine, asparagine, aspartic acid, cysteine, glutamic acid, glutamine, glycine, histidine, isoleucine, leucine, lysine, methionine, phenylalanine, proline, serine, threonine, tryptophan, tyrosine, valine, δ-aminolevulinic acid, 4-aminobenzoic acid, and γ-aminobutyric acid. In some embodiments, the amino acid is the L isomer. In some embodiments, the amino acid is the D isomer. In some embodiments, the amino acid is an α-amino acid. In some embodiments, the amino acid is a β-, γ-, or δ-amino acid. For example, a β-amino acid can be β-alanine, β-leucine, β-lysine, β-arginine, β-glutamate, β-β-glutamine, 3-phenylalanine, and β-tyrosine.

In some embodiments, the one or more amines are present in an amount of about 0.5% to about 2%. For example, about 0.5%, to about 0.75%, about 0.5% to about 1%, about 0.5% to about 1.25%, about 0.5% to about 1.5%, about 0.5% to about 1.75%, about 1.75% to about 2%, about 1.5% to about 2%, about 1.25% to about 2%, about 1% to about 2%, or about 0.75% to about 2% w/w of the composition. In some embodiments, the one or more amines are present in an amount of about 0.5% to about 0.6%, about 0.55% to about 0.65%, about 0.6% to about 0.7%, about 0.65% to about 0.75%, about 0.7% to about 0.8%, about 0.75% to about 0.85%, about 0.8% to about 0.9%, about 0.85% to about 0.95%, about 0.9% to about 1%, about 0.95% to about 1.05%, about 1% to about 1.1%, about 1.05% to about 1.15%, about 1.1% to about 1.2%, about 1.15% to about 1.25%, about 1.2% to about 1.3%, about 1.25% to about 1.35%, about 1.3% to about 1.4%, about 1.35% to about 1.45%, about 1.4% to about 1.6%, about 1.5% to about 1.7%, about 1.6% to about 1.8%, or about 1.7% to about 1.9% w/w of the composition. For example, about 0.5%, about 0.55%, about 0.6%, about 0.65%, about 0.7%, about 0.72%, about 0.74%, about 0.76%, about 0.78%, about 0.8%, about 0.81%, about 0.82%, about 0.83%, about 0.84%, about 0.85%, about 0.86%, about 0.87%, about 0.88%, about 0.89%, about 0.9%, about 0.91%, about 0.92%, about 0.93%, about 0.94%, about 0.96%, about 0.98%, about 1%, about 1.05%, about 1.1%, about 1.15%, about 1.2%, about 1.25%, about 1.3%, about 1.35%, about 1.4%, about 1.5%, about 1.6%, about 1.7%, about 1.8%, about 1.9%, or about 2% w/w of the composition.

In some embodiments, the one or more amines are selected from the group consisting of: an amino acid, panthenol, triethanolamine, and other compounds with primary, secondary or tertiary amines.

In some embodiments, the one or more amines is one or more amino acids. In some embodiments, the one or more amines comprise L-arginine. In some embodiments, the one or more amines is L-arginine. In some embodiments, L-arginine is present in an amount of about 0.5% to about 2%. For example, about 0.5%, to about 0.75%, about 0.5% to about 1%, about 0.5% to about 1.25%, about 0.5% to about 1.5%, about 0.5% to about 1.75%, about 1.75% to about 2%, about 1.5% to about 2%, about 1.25% to about 2%, about 1% to about 2%, or about 0.75% to about 2% w/w of the composition. In some embodiments, the L-arginine is present in an amount of about 0.5% to about 0.6%, about 0.55% to about 0.65%, about 0.6% to about 0.7%, about 0.65% to about 0.75%, about 0.7% to about 0.8%, about 0.75% to about 0.85%, about 0.8% to about 0.9%, about 0.85% to about 0.95%, about 0.9% to about 1%, about 0.95% to about 1.05%, about 1% to about 1.1%, about 1.05% to about 1.15%, about 1.1% to about 1.2%, about 1.15% to about 1.25%, about 1.2% to about 1.3%, about 1.25% to about 1.35%, about 1.3% to about 1.4%, about 1.35% to about 1.45%, about 1.4% to about 1.6%, about 1.5% to about 1.7%, about 1.6% to about 1.8%, or about 1.7% to about 1.9% w/w of the composition. For example, about 0.5%, about 0.55%, about 0.6%, about 0.65%, about 0.7%, about 0.72%, about 0.74%, about 0.76%, about 0.78%, about 0.8%, about 0.81%, about 0.82%, about 0.83%, about 0.84%, about 0.85%, about 0.86%, about 0.87%, about 0.88%, about 0.89%, about 0.9%, about 0.91%, about 0.92%, about 0.93%, about 0.94%, about 0.96%, about 0.98%, about 1%, about 1.05%, about 1.1%, about 1.15%, about 1.2%, about 1.25%, about 1.3%, about 1.35%, about 1.4%, about 1.5%, about 1.6%, about 1.7%, about 1.8%, about 1.9%, or about 2% w/w of the composition.

A “humectant” as used herein refers to a substance that attracts water. For example, a humectant may attract water to bring moisture to the skin. Non-limiting examples of a humectant include glycerin, propylene glycol, a polyethylene glycol, a sugar, hexylene glycol, butylene glycol, aloe vera gel, an alpha hydroxy acid such as lactic acid, glyceryl triacetate, lithium chloride, sorbitol, xylitol, maltitol, hyaluronic acid, allantoin, urea, tremella extract, dicyanamide, sodium lactate, sodium L-pyroglutamate urea, and pyrrolidone carboxylic acid.

In some embodiments, the one or more humectants are present in an amount of about 1% to about 10% w/w of the composition. For example, about 1% to about 2%, about 1% to about 3%, about 1% to about 4%, about 1% to about 5%, about 1% to about 6%, about 1% to about 7%, about 1% to about 8%, about 1% to about 9%, about 9% to about 10%, about 8% to about 10%, about 7% to about 10%, about 6% to about 10%, about 5% to about 10%, about 4% to about 10%, about 3% to about 10%, about 2% to about 10% w/w of the composition. In some embodiments, the one or more humectants are present in an amount of about 1% to about 3%, about 2% to about 4%, about 3% to about 5%, about 4% to about 6%, about 5% to about 7%, about 6% to about 8%, or about 7% to about 9% w/w of the composition. For example, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% w/w of the composition.

In some embodiments, non-limiting examples of the one or more humectants include: glycerin, propylene glycol, a polyethylene glycol, a sugar, hexylene glycol, butylene glycol, Aloe vera gel, an alpha hydroxy acid such as lactic acid, glyceryl triacetate, lithium chloride, sorbitol, xylitol, maltitol, hyaluronic acid, allantoin, urea, tremella extract, dicyanamide, sodium lactate, and sodium L-pyroglutamate.

In some embodiments, the one or more humectants comprise glycerin. In some embodiments, the one or more humectants is glycerin. In some embodiments, glycerin is present in an amount of about 1% to about 10% w/w of the composition. For example, about 1% to about 2%, about 1% to about 3%, about 1% to about 4%, about 1% to about 5%, about 1% to about 6%, about 1% to about 7%, about 1% to about 8%, about 1% to about 9%, about 9% to about 10%, about 8% to about 10%, about 7% to about 10%, about 6% to about 10%, about 5% to about 10%, about 4% to about 10%, about 3% to about 10%, about 2% to about 10% w/w of the composition. In some embodiments, the glycerin is present in an amount of about 1% to about 3%, about 2% to about 4%, about 3% to about 5%, about 4% to about 6%, about 5% to about 7%, about 6% to about 8%, or about 7% to about 9% w/w of the composition. For example, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% w/w of the composition.

In some embodiments, the skin priming composition can further comprise one or more semi-volatile semi-permanent colorant solubilizers. In some embodiments, the one or more semi-volatile semi-permanent colorant solubilizers are present in an amount of about 0.1% to about 10% w/w of the composition. For example, about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2%, about 0.1% to about 1%, about 9% to about 10%, about 8% to about 10%, about 7% to about 10%, about 6% to about 10%, about 5% to about 10%, about 4% to about 10%, about 3% to about 10%, about 2% to about 10%, or about 1% to about 10% w/w of the composition. In some embodiments, the one or more semi-volatile semi-permanent colorant solubilizers are present in an amount of about 0.5% to about 1%, about 0.5% to about 1.5%, about 1% to about 2%, about 1.5% to about 2.5%, about 2% to about 3%, or about 0.5% to about 3% w/w of the composition. In some embodiments, the one or more semi-volatile semi-permanent colorant solubilizers are present in an amount of about 0.25%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% w/w of the composition.

In some embodiments, the one or more semi-volatile semi-permanent colorant solubilizers are selected from the group consisting of: a polyethylene glycol, an alkyl glycol, and an alkylene glycol ether.

In some embodiments, the semi-volatile semi-permanent colorant solubilizer comprises diethylene glycol monoethyl ether. In some embodiments, the semi-volatile semi-permanent colorant solubilizer is diethylene glycol monoethyl ether.

In some embodiments, the skin priming composition can further comprise one or more skin penetration enhancers. In some embodiments, the one or more skin penetration enhancers are present in an amount of about 5% to about 10% w/w of the composition. For example, about 5% to about 6%, about 5% to about 7%, about 5% to about 8%, about 5% to about 9%, about 9% to about 10%, about 8% to about 10%, about 7% to about 10%, or about 6% to about 10% w/w of the composition. In some embodiments, the one or more skin penetration enhancers are present in an amount of about 5% to about 7%, about 6% to about 8%, or about 7% to about 9% w/w of the composition. For example, about 5%, about 5.25%, about 5.5%, about 5.75%, about 6%, about 6.25%, about 6.5%, about 6.75%, about 7%, about 7.25%, about 7.5%, about 7.75%, about 8%, about 8.25%, about 8.5%, about 8.75%, about 9%, about 9.25%, about 9.5%, about 9.75%, or about 10% w/w of the composition.

In some embodiments, non-limiting examples of the one or more skin penetration enhancers can include: an alcohol, an amide, an ester, an ether alcohol, a fatty acid, a glycol, a pyrrolidone, a sulphoxide, a surfactant, and a terpene. In some embodiments, non-limiting examples of the one or more skin penetration enhancers include: ethanol, isopropanol, decanol, hexanol, lauryl alcohol, myristyl alcohol, octanol, octyl dodecanol, oleyl alcohol, azone, ethyl acetate, octyl salicylate, padimate O, ethyl oleate, glyceryl monoleate, glyceryl monocaprate, glyceryl tricaprylate, isopropyl myristate, isopropyl palmitate, propylene glycol monolaurate, propylene glycol monocaprylate, diethylene glycol monoethyl ether (TRANSCUTOL®), lauric acid, linoleic acid, linolenic acid, myristic acid, oleic acid, palmitic acid, stearic acid, isostearic acid, dipropylene glycol, propylene glycol, 1,2-butylene glycol, 1,3-butylene glycol, n-methyl-2-pyrrolidone, 2-pyrrolidone, decylmethyl sulphoxide, dimethyl sulphoxide, sodium lauryl sulphate, alkyl dimethylbenzyl ammonium halides, alkyl trimethyl ammonium halides, alkyl pyridinium halides, brij 36t, polyethylene glycol sorbitan monooleate (e.g., TWEEN® 80), eugenol, D-limonene, menthol, menthone, farnesol, and neridol.

In some embodiments, a skin priming composition can further comprise one or more preservatives. In some embodiments, the one or more preservatives are present in an amount of about 0.05% to about 5% w/w of the composition. For example, about 0.05% to about 0.1%, about 0.05% to about 0.5%, about 0.05% to about 1%, about 0.05% to about 2%, about 0.05% to about 3%, about 0.05% to about 4%, about 4% to about 5%, about 3% to about 5%, about 2% to about 5%, about 1% to about 5%, about 0.5% to about 5%, or about 0.1% to about 5% w/w of the composition. In some embodiments, the one or more preservatives are present in an amount of 0.1% to about 2%, 0.5% to about 2.5%, about 1% to about 3%, about 1.5% to about 3.5%, about 2% to about 4%, about 2.5% to about 4.5% w/w of the composition. In some embodiments, the one or more preservatives are present in an amount of about 0.5% to about 2% w/w of the composition. In some embodiments, the one or more preservatives are present in an amount of about 0.5% to about 1.5%, about 0.5% to about 1.0%, about 0.05% to about 0.5%, about 0.05% to about 0.1%, about 1.5% to about 2%, about 1% to about 2%, about 0.5% to about 2%, or about 0.8% to about 2%, or about 0.1% to about 0.9% w/w of the composition. For example, about 0.05% to about 0.4%, about 0.05% to about 0.2%, about 0.2% to about 0.5%, about 0.15% to about 0.25%, or about 0.1% to about 0.3% w/w of the composition. In some embodiments, the one or more preservatives are present in an amount of about 0.01%, about 0.05%, about 0.1%, about 0.12%, about 0.14%, about 0.15%, about 0.16%, about 0.18%, about 0.2%, about 0.22% about 0.25%, about 0.26%, about 0.28%, about 0.3%, about 0.32%, about 0.34%, about 0.35%, about 0.36%, about 0.38%, about 0.4%, about 0.45%, about 0.5%, about 0.6%, about 0.65%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, or about 2% w/w of the composition.

In some embodiments, the one or more preservatives are selected form the group consisting of: ascorbic acid, an ascorbate, a palmitate, citric acid, a benzoate, a benzoic acid, a propionate, propionic acid, a sorbate, sorbic acid, a salicylic acid, a salicylate, hexa-2,4-dienoic acid, a hexa-2,4-dienoate, formaldehyde, a formaldehyde releaser, formic acid and its salts, 3-acetyl-6-methylpyran-2,4-(3H)-dione and its salts, 3,3′-dibromo-4,4′-hexamethylenedioxydibenzamidine and its salts, thiomersal, phenylmercuric salts, undec-10-enoic acid and its salts, 1,3-bis (2-ethylhexyl) hexahydro-5-methyl-5-pyrimidine, 5-bromo-5-nitro-1,3-dioxane, bronopol, 2,4-dichlorobenzyl alcohol, 1-(4-chlorophenyl)-3-(3,4-dichlorophenyl) urea, chlorocresol, chloroxylenol, 5-chloro-2-(2,4-dichlorophenoxy) phenol, N,N″-methylenebis[N′-[3-(hydroxymethyl)-2,5-dioxoimidazolidin-4-yl]urea], polyaminopropyl biguanide, methenamine, quaternium-15, climbazole, DMDM hydantoin, benzyl alcohol, 1-Hydroxy-4-methyl-6-(2,4,4-trimethylpentyl)-2-pyridon, piroctone olamine, bromochlorophene, o-cymen-5-ol, chlorophene, chloroacetaminde, methylchloroisothiazolinone, methylisothiazolinone, phenoxyisopropanol, chlorhexidine, chlorhexidine diacetate, chlorhexidine digluconate, chlorhexidine dihydrochloride, dimethyl oxazolidine, behentrimonium chloride, cetri-monium bromide, cetrimonium chloride, laurtrimonium bromide, laurtrimonium chloride, steartrimonium bromide, steartrimonium chloride, diazolidinyl urea, hexamidine, hexamidine diisethionate, hexamidine paraben, glutaral, 7-ethylbicyclooxazolidine, chlorphenesin, sodium hydroxymethylglycinate, silver chloride, benzethonium chloride, benzalkonium chloride, benza-lkonium bromide, benzalkonium saccharinate, benzylhemiformal, iodopropynyl butylcarbamate, biphenyl-2-ol and its salts, pyrithionine zinc, an erythorbate, a nitrite, ethylenediaminetetraacetic acid (EDTA), butylated hydroxyanisole (BHA), butylated hydroxytoluene (BHT), capryllic acid, dilauryl thiodipropionate, erythorbic acid, gum guaiac, methylparaben, a sulfite, a bisulfite, a metabisulfite, propyl gallatepy, propylparaben, stannous chloride, sulfur dioxide, thiodipropionic acid, an isothiazoline, a paraben, phenoxyethanol, ethylhexylglycerin, a glycols, and a tocopherol.

In some embodiments, the one or more preservatives are phenoxyethanol and ethylhexylglycerin (e.g., Euxyl® PE9010). In some embodiments, the phenoxyethanol and ethylhexylglycerin are present in the composition at a ratio of 9:1 phenoxyethanol:ethylhexylglycerin.

In some embodiments, phenoxyethanol and ethylhexylglycerin are present in an amount of about 0.05% to about 5% w/w of the composition. For example, about 0.05% to about 0.1%, about 0.05% to about 0.5%, about 0.05% to about 1%, about 0.05% to about 2%, about 0.05% to about 3%, about 0.05% to about 4%, about 4% to about 5%, about 3% to about 5%, about 2% to about 5%, about 1% to about 5%, about 0.5% to about 5%, or about 0.1% to about 5% w/w of the composition. In some embodiments, phenoxyethanol and ethylhexylglycerin are present in an amount of 0.1% to about 2%, 0.5% to about 2.5%, about 1% to about 3%, about 1.5% to about 3.5%, about 2% to about 4%, about 2.5% to about 4.5% w/w of the composition. In some embodiments of the ink compositions described herein, phenoxyethanol and ethylhexylglycerin are present in an amount of about 0.5% to about 2% w/w of the composition. In some embodiments of the ink compositions described herein, phenoxyethanol and ethylhexylglycerin are present in an amount of about 0.5% to about 1.5%, about 0.5% to about 1.0%, about 0.05% to about 0.5%, about 0.05% to about 0.1%, about 1.5% to about 2%, about 1% to about 2%, about 0.5% to about 2%, or about 0.8% to about 2%, or about 0.1% to about 0.9% w/w of the composition. For example, about 0.05% to about 0.4%, about 0.05% to about 0.2%, about 0.2% to about 0.5%, about 0.15% to about 0.25%, or about 0.1% to about 0.3% w/w of the composition. In some embodiments of the ink compositions described herein, phenoxyethanol and ethylhexylglycerin are present in an amount of about 0.01%, about 0.05%, about 0.1%, about 0.12%, about 0.14%, about 0.15%, about 0.16%, about 0.18%, about 0.2%, about 0.22% about 0.25%, about 0.26%, about 0.28%, about 0.3%, about 0.32%, about 0.34%, about 0.35%, about 0.36%, about 0.38%, about 0.4%, about 0.45%, about 0.5%, about 0.6%, about 0.65%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, or about 2% w/w of the composition.

In some embodiments, a skin priming composition comprises:

one or more volatile solvents present in an amount of about 91% to about 97% w/w of the composition;

one or more amines present in an amount of about 0.8% to about 1.2% w/w of the composition;

and one or more humectants present in an amount of about 1% to about 10% w/w of the composition. In some embodiments, the composition further comprises one or more preservatives present in an amount of about 0.1% to about 2% w/w of the composition.

In some embodiments, a skin priming composition comprises:

one or more volatile solvents present in an amount of about 93% w/w of the composition;

one or more amines present in an amount of about 1% w/w of the composition;

and one or more humectants present in an amount of about 5% w/w of the composition. In some embodiments, the composition further comprises one or more preservatives present in an amount of about 1% w/w of the composition.

In some embodiments, a skin priming composition comprises:

one or more volatile solvents present in an amount of about 91% to about 97% w/w of the composition, wherein the one or more volatile solvents is water;

one or more amines present in an amount of about 0.8% to about 1.2% w/w of the composition, wherein the one or more amines is L-arginine;

and one or more humectants present in an amount of about 1% to about 10% w/w of the composition, wherein the one or more humectants is glycerin. In some embodiments, the composition further comprises one or more preservatives present in an amount of about 0.1% to about 2% w/w of the composition, wherein the one or more preservatives are phenoxyethanol and ethylhexylglycerin (e.g., phenoxyethanol and ethylhexylglycerin at about a 9:1 ratio).

In some embodiments, a skin priming composition comprises:

one or more volatile solvents present in an amount of about 91% to about 97% w/w of the composition, wherein the one or more volatile solvents is water;

one or more amines present in an amount of about 0.9% to about 1.1% w/w of the composition, wherein the one or more amines is L-arginine;

and one or more humectants present in an amount of about 1% to about 10% w/w of the composition, wherein the one or more humectants is glycerin. In some embodiments, the composition further comprises one or more preservatives present in an amount of about 0.1% to about 2% w/w of the composition, wherein the one or more preservatives are phenoxyethanol and ethylhexylglycerin (e.g., phenoxyethanol and ethylhexylglycerin at about a 9:1 ratio).

In some embodiments, a skin priming composition comprises:

one or more volatile solvents present in an amount of about 93% w/w of the composition, wherein the one or more volatile solvents is water;

one or more amines present in an amount of about 1% w/w of the composition, wherein the one or more amines is L-arginine;

and one or more humectants present in an amount of about 5% w/w of the composition, wherein the one or more humectants is glycerin. In some embodiments, the composition further comprises one or more preservatives present in an amount of about 1% w/w of the composition, wherein the one or more preservatives are phenoxyethanol and ethylhexylglycerin (e.g., phenoxyethanol and ethylhexylglycerin at about a 9:1 ratio).

In some embodiments, a skin priming composition comprises:

one or more volatile solvents present in an amount of about 91% to about 97% w/w of the composition;

one or more amines present in an amount of about 0.8% to about 0.9% w/w of the composition;

and one or more humectants present in an amount of about 1% to about 10% w/w of the composition.

In some embodiments, a skin priming composition comprises:

one or more volatile solvents present in an amount of about 91% to about 97% w/w of the composition, wherein the one or more volatile solvents is water;

one or more amines present in an amount of about 0.8% to about 0.9% w/w of the composition, wherein the one or more amines is L-arginine;

and one or more humectants present in an amount of about 1% to about 10% w/w of the composition, wherein the one or more humectants is glycerin.

In some embodiments, a skin priming composition comprises:

one or more volatile solvents present in an amount of about 94% w/w of the composition, wherein the one or more volatile solvents is water;

one or more amines present in an amount of about 0.85% w/w of the composition, wherein the one or more amines is L-arginine;

and one or more humectants present in an amount of about 5% w/w of the composition, wherein the one or more humectants is glycerin.

In some embodiments, a skin priming composition can be configured to have a pH of about 4 to about 7. For example, about 4 to about 5, about 4.5 to about 5.5, about 5 to about 6, about 5.5 to about 6.5, or about 6 to about 7. In some embodiments, skin priming composition can be configured to have a pH of a pH of about 4, about 4.25, about 4.5, about 4.75, about 5, about 5.25, about 5.5, about 5.75, about 6, about 6.25, about 6.5, about 6.75, or about 7.

In some embodiments, a skin priming composition is adjusted to a pH of about 4 to about 7. For example, about 4 to about 5, about 4.5 to about 5.5, about 5 to about 6, about 5.5 to about 6.5, or about 6 to about 7. In some embodiments, skin priming composition is adjusted to a pH of a pH of about 4, about 4.25, about 4.5, about 4.75, about 5, about 5.25, about 5.5, about 5.75, about 6, about 6.25, about 6.5, about 6.75, or about 7.

In some embodiments, a skin priming composition can have a pH of about 4 to about 7. For example, about 4 to about 5, about 4.5 to about 5.5, about 5 to about 6, about 5.5 to about 6.5, or about 6 to about 7. In some embodiments, skin priming composition can have a pH of a pH of about 4, about 4.25, about 4.5, about 4.75, about 5, about 5.25, about 5.5, about 5.75, about 6, about 6.25, about 6.5, about 6.75, or about 7.

Also provided herein is a skin priming article moistened with the skin priming composition of any of the above embodiments. In some embodiments, the moistened article can be a towelette, a pad, a woven, a nonwoven, or the like. In some embodiments, the moistened article can be contained within a sealed package in order to maintain the moist nature of the article.

Also provided herein are aftercare compositions comprising one or more volatile solvents, one or more humectants, and one or more occlusive agents. Aftercare compositions can be used, for instance, to prolong the duration of a semi-permanent tattoo. For example, aftercare compositions as described herein may help to slow down the rate of sloughing of skin, or shedding of dead skin surface cells. In some embodiments, aftercare compositions as described herein may help to maintain the health of skin surface cells, and slow down the turnover, aging, and/or death of skin surface cells.

In some embodiments, the one or more volatile solvents are present in the aftercare composition in an amount of about 20% to about 90% w/w of the composition. For example, about 20% to about 60%, about 50% to about 90%, about 20% to about 40%, about 30% to about 50%, about 40% to about 60%, about 50% to about 70%, about 60% to about 80%, or about 70% to about 90% w/w of the composition. In some embodiments, the one or more volatile solvents are present in the aftercare composition in an amount of about 30% to about 35%, about 32% to about 37%, about 35% to about 40%, about 37% to about 42%, about 40% to about 50%, about 50% to about 60%, about 60% to about 65%, about 62% to about 67%, about 65% to about 70%, about 67% to about 72%, about 70% to about 75%, about 75% to about 80%, about 80% to about 85%, or about 85% to about 90% w/w of the composition. In some embodiments, the one or more volatile solvents are present in the aftercare composition in an amount of about 20%, about 21%, about 22%, about 23%, about 24%, about 25%, about 25%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77% w/w, about 78%, about 79%, about 80%, about 81%, about 82%, about 83%, about 84%, about 85%, about 86%, about 87%, about 88%, about 89%, or about 90% of the composition.

In some embodiments, non-limiting examples of the one or more volatile solvents in the skin priming composition include: water, methanol, ethanol, and isopropanol.

In some embodiments, the one or more humectants are present in an amount of about 5% to about 45% w/w of the composition. For example, about 5% to about 10%, about 5% to about 15%, about 5% to about 20%, about 5% to about 25%, about 5% to about 30%, about 5% to about 35%, about 5% to about 40%, about 40% to about 45%, about 35% to about 45%, about 30% to about 45%, about 25% to about 45%, about 20% to about 45%, about 15% to about 45%, or about 10% to about 40% w/w of the composition. In some embodiments, the one or more humectants are present in an amount of about 5% to about 15%, about 10% to about 25%, about 15% to about 35%, about 20% to about 30%, about 25% to about 35%, or about 30% to about 40% w/w of the composition. For example, about 20%, about 21%, about 22%, about 23%, about 24%, about 25%, about 26%, about 27%, about 28%, about 29%, about 30%, about 31%, about 32%, about 33%, about 34%, or about 35% w/w of the composition.

In some embodiments, non-limiting examples of the one or more humectants include: glycerin, propylene glycol, a polyethylene glycol, a sugar, hexylene glycol, butylene glycol, aloe vera gel, an alpha hydroxy acid such as lactic acid, glyceryl triacetate, lithium chloride, sorbitol, xylitol, maltitol, hyaluronic acid, allantoin, urea, tremella extract, dicyanamide, sodium lactate, sodium L-pyroglutamate, urea, and pyrrolidone carboxylic acid.

In some embodiments, the one or more humectants comprise glycerin. In some embodiments, the one or more humectants is glycerin. In some embodiments, the glycerin is present in an amount of about 5% to about 45% w/w of the composition. For example, about 5% to about 10%, about 5% to about 15%, about 5% to about 20%, about 5% to about 25%, about 5% to about 30%, about 5% to about 35%, about 5% to about 40%, about 40% to about 45%, about 35% to about 45%, about 30% to about 45%, about 25% to about 45%, about 20% to about 45%, about 15% to about 45%, or about 10% to about 40% w/w of the composition. In some embodiments, the glycerin is present in an amount of about 5% to about 15%, about 10% to about 25%, about 15% to about 35%, about 20% to about 30%, about 25% to about 35%, or about 30% to about 40% w/w of the composition. For example, about 20%, about 21%, about 22%, about 23%, about 24%, about 25%, about 26%, about 27%, about 28%, about 29%, about 30%, about 31%, about 32%, about 33%, about 34%, or about 35% w/w of the composition.

An “occlusive agent” as used herein refers to an agent that physically prevents or reduces water loss. For example, an occlusive agent may provide a barrier that prevents or reduces trans-epidermal water loss. Non-limiting examples of occlusive agents include a film former, a silicone, a wax, an oil, and petrolatum. For example, a film former may include a vinylpyrrolidone, an acrylate, a silsequioxane, a siloxane, a vinyl alcohol, a glycol, urethane, styrene, butadiene, a peptide, or a polysaccharide.

An “emollient” as used herein refers to an agent that softens or soothes the skin by filling spaces between skin flakes and creating a smooth skin surface. Non-limiting examples of an emollient include an alkyl alcohol, a glycol alkyl ether, a short chain hydrocarbon, and an alkyl ester. Other non-limiting examples of an emollient include collagen, colloidal oatmeal, elastin, glyceryl stearate, isopropyl palmitate, shea butter, and stearic acid. In some embodiments, at least one of the one or more emollients also function as an occlusive agent (also referred to as an occlusive emollient herein). For example, a combination of dimethicone, trisiloxane, ceteth-10, laureth-4 (e.g., Dowsil 7-3110 Volatile Fluid HIP Emulsion) is an emollient that has occlusive properties.

In some embodiments, the one or more occlusive agents are present in an amount of about 0.05% to about 2% w/w of the composition. For example, about 0.05% to about 0.2%, about 0.05% to about 0.3%, about 0.05% to about 0.4%, about 0.05% to about 0.5%, about 0.15% to about 0.45%, about 0.2% to about 0.4%, about 0.4% to about 0.5%, about 0.3% to about 0.5%, about 0.2% to about 0.5%, or about 0.1% to about 0.5% w/w of the composition. In some embodiments, the one or more occlusive agents are present in an amount of about 0.05% to about 0.1%, about 0.075% to about 0.125% about 0.1% to about 0.15%, about 0.125% to about 0.175%, about 0.15% to about 0.2%, about 0.175% to about 0.225%, about 0.2% to about 0.25%, about 0.225% to about 0.275%, about 0.25% to about 0.3%, about 0.275% to about 0.325%, about 0.3% to about 0.35%, about 0.325% to about 0.375%, about 0.35% to about 0.4%, about 0.375% to about 0.425%, about 0.4% to about 0.45%, about 0.425% to about 0.475%, about 0.45% to about 0.5%, about 0.475% to about 0.525%, about 0.5% to about 0.55%, about 0.525% to about 0.575%, about 0.55% to about 0.6%, about 0.6% to about 0.7%, about 0.7% to about 0.8%, about 0.8% to about 0.9%, about 0.9% to about 1%, about 1% to about 1.2%, about 1.2% to about 1.4%, about 1.4% to about 1.6%, about 1.6% to about 1.8%, or about 1.8% to about 2% w/w of the composition. For example, about 0.05%, about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1%, about 1.1%, about 1.2%, about 1.3%, about 1.4%, about 1.5%, about 1.6%, about 1.7%, about 1.8%, about 1.9%, about 2% w/w of the composition.

In some embodiments, the one or more occlusive agents comprises an acrylate/C10-30alkyl acrylate crosspolymer. In some embodiments, the one or more occlusive agents is an acrylate/C10-30alkyl acrylate crosspolymer. In some embodiments, the acrylate/C10-30alkyl acrylate crosspolymer is present in an amount of about 0.05% to about 2% w/w of the composition. For example, about 0.05% to about 0.2%, about 0.05% to about 0.3%, about 0.05% to about 0.4%, about 0.05% to about 0.5%, about 0.15% to about 0.45%, about 0.2% to about 0.4%, about 0.4% to about 0.5%, about 0.3% to about 0.5%, about 0.2% to about 0.5%, or about 0.1% to about 0.5% w/w of the composition. In some embodiments, the acrylate/C10-30alkyl acrylate crosspolymer is present in an amount of about 0.05% to about 0.1%, about 0.075% to about 0.125% about 0.1% to about 0.15%, about 0.125% to about 0.175%, about 0.15% to about 0.2%, about 0.175% to about 0.225%, about 0.2% to about 0.25%, about 0.225% to about 0.275%, about 0.25% to about 0.3%, about 0.275% to about 0.325%, about 0.3% to about 0.35%, about 0.325% to about 0.375%, about 0.35% to about 0.4%, about 0.375% to about 0.425%, about 0.4% to about 0.45%, about 0.425% to about 0.475%, about 0.45% to about 0.5%, about 0.475% to about 0.525%, about 0.5% to about 0.55%, about 0.525% to about 0.575%, about 0.55% to about 0.6%, about 0.6% to about 0.7%, about 0.7% to about 0.8%, about 0.8% to about 0.9%, about 0.9% to about 1%, about 1% to about 1.2%, about 1.2% to about 1.4%, about 1.4% to about 1.6%, about 1.6% to about 1.8%, or about 1.8% to about 2% w/w of the composition. For example, about 0.05%, about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1%, about 1.1%, about 1.2%, about 1.3%, about 1.4%, about 1.5%, about 1.6%, about 1.7%, about 1.8%, about 1.9%, about 2% w/w of the composition.

In some embodiments, the composition further comprises an emollient. In some embodiments, the one or more emollients are present in an amount of about 5% to about 55% w/w of the composition. For example, about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 50% to about 55%, about 45% to about 55%, about 40% to about 55%, about 35% to about 5%, about 30% to about 55%, about 25% to about 55%, about 20% to about 55%, about 20% to about 55%, about 15% to about 55%, or about 10% to about 55% w/w of the composition. For example, about 10% to about 20%, about 15% to about 25%, about 20% to about 30%, about 25% to about 35%, about 25% to about 30%, about 30% to about 40%, about 30% to about 35%, about 35% to about 45%, about 35% to about 40%, or about 40% to about 50% w/w of the composition. In some embodiments, the one or more emollients are present in an amount of about 25%, about 26%, about 27%, about 28%, about 29%, about 30%, about 31%, about 32%, about 33%, about 34%, about 35%, about 36%, about 3′7%, about 38%, about 39%, about 40%, about 41%, about 42%, about 43%, about 44%, about 45%, about 46%, about 47%, about 48%, about 49%, or about 50% w/w of the composition.

In some embodiments, the one or more emollients are selected from the group consisting of: an alkyl alcohol, a glycol alkyl ether, a short chain hydrocarbon, and an alkyl ester. In some embodiments, the one or more emollients are selected from the group consisting of: collagen, colloidal oatmeal, elastin, glyceryl stearate, isopropyl palmitate, shea butter, and stearic acid. In some embodiments, at least one of the one or more emollients also function as an occlusive agent.

In some embodiments, wherein at least one of the one or more emollients also function as an occlusive agent, the at least one emollient that also functions as an occlusive agent is a combination of dimethicone, trisiloxane, ceteth-10, and laureth-4 (e.g., Dowsil 7-3110 Volatile Fluid HIP Emulsion). In some embodiments, the combination of dimethicone, trisiloxane, ceteth-10, and laureth-4 are present in an amount of about 5% to about 55% w/w of the composition. For example, about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 50% to about 55%, about 45% to about 55%, about 40% to about 55%, about 35% to about 5%, about 30% to about 55%, about 25% to about 55%, about 20% to about 55%, about 20% to about 55%, about 15% to about 55%, or about 10% to about 55% w/w of the composition. For example, about 10% to about 20%, about 15% to about 25%, about 20% to about 30%, about 25% to about 35%, about 30% to about 40%, or about 35% to about 45% w/w of the composition. In some embodiments, the combination of dimethicone, trisiloxane, ceteth-10, and laureth-4 are present in an amount of about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50% w/w of the composition.

In some embodiments, an aftercare composition as described herein can further include one or more amines. In some embodiments, the one or more amines are present in an amount of about 0.01% to about 2% w/w of the composition. For example, about 0.01%, to about 0.05%, about 0.01%, to about 0.1%, about 0.01%, to about 0.25%, about 0.01%, to about 0.5%, about 0.01%, to about 0.75%, about 0.01% to about 1%, about 0.01% to about 1.25%, about 0.01% to about 1.5%, about 0.01% to about 1.75%, about 0.01% to about 2%, about 1.75% to about 2%, about 1.5% to about 2%, about 1.25% to about 2%, about 1% to about 2%, about 0.75% to about 2%, about 0.5% to about 2%, about 0.25% to about 2%, about 0.1% to about 2%, about 0.05% to about 2% w/w of the composition. In some embodiments, the one or more amines are present in an amount of 0.25% to about 1.25%, about 0.2% to about 0.3%, about 0.25% to about 0.35%, about 0.3% to about 0.4%, about 0.35% to about 0.45%, about 0.4% to about 0.5%, about 0.45% to about 0.55%, about 0.5% to about 0.6%, about 0.55% to about 0.65%, about 0.6% to about 0.7%, about 0.65% to about 0.75%, about 0.7% to about 0.8%, about 0.75% to about 0.85%, about 0.8% to about 0.9%, about 0.85% to about 0.95%, about 0.9% to about 1%, about 0.95% to about 1.05%, about 1% to about 1.1%, about 1.05% to about 1.15%, about 1.1% to about 1.2%, about 1.15% to about 1.25%, about 1.2% to about 1.3%, about 1.25% to about 1.35%, about 1.3% to about 1.4%, about 1.35% to about 1.45%, about 1.4% to about 1.6%, about 1.5% to about 1.7%, about 1.6% to about 1.8%, or about 1.7% to about 1.9% w/w of the composition. For example, about 0.25%, about 0.3%, about 0.35%, about 0.4%, about 0.45%, about 0.5%, about 0.55%, about 0.6%, about 0.65%, about 0.7%, about 0.72%, about 0.74%, about 0.76%, about 0.78%, about 0.8%, about 0.81%, about 0.82%, about 0.83%, about 0.84%, about 0.85%, about 0.86%, about 0.87%, about 0.88%, about 0.89%, about 0.9%, about 0.91%, about 0.92%, about 0.93%, about 0.94%, about 0.96%, about 0.98%, about 1%, about 1.05%, about 1.1%, about 1.15%, about 1.2%, about 1.25%, about 1.3%, about 1.35%, about 1.4%, about 1.5%, about 1.6%, about 1.7%, about 1.8%, about 1.9%, or about 2% w/w of the composition.

In some embodiments, the one or more amines are selected from the group consisting of: an amino acid, panthenol, triethanolamine, and other compounds with primary, secondary or tertiary amines. In some embodiments, the one or more amines is one or more amino acids.

In some embodiments, the one or more amines comprise L-arginine. In some embodiments, the one or more amines is L-arginine. In some embodiments, L-arginine is present in an amount of about 0.01% to about 2% w/w of the composition. For example, about 0.01%, to about 0.05%, about 0.01%, to about 0.1%, about 0.01%, to about 0.25%, about 0.01%, to about 0.5%, about 0.01%, to about 0.75%, about 0.01% to about 1%, about 0.01% to about 1.25%, about 0.01% to about 1.5%, about 0.01% to about 1.75%, about 0.01% to about 2%, about 1.75% to about 2%, about 1.5% to about 2%, about 1.25% to about 2%, about 1% to about 2%, about 0.75% to about 2%, about 0.5% to about 2%, about 0.25% to about 2%, about 0.1% to about 2%, about 0.05% to about 2% w/w of the composition. In some embodiments, the L-arginine is present in an amount of about 0.25% to about 1.25%, 0.2% to about 0.3%, about 0.25% to about 0.35%, about 0.3% to about 0.4%, about 0.35% to about 0.45%, about 0.4% to about 0.5%, about 0.45% to about 0.55%, about 0.5% to about 0.6%, about 0.55% to about 0.65%, about 0.6% to about 0.7%, about 0.65% to about 0.75%, about 0.7% to about 0.8%, about 0.75% to about 0.85%, about 0.8% to about 0.9%, about 0.85% to about 0.95%, about 0.9% to about 1%, about 0.95% to about 1.05%, about 1% to about 1.1%, about 1.05% to about 1.15%, about 1.1% to about 1.2%, about 1.15% to about 1.25%, about 1.2% to about 1.3%, about 1.25% to about 1.35%, about 1.3% to about 1.4%, about 1.35% to about 1.45%, about 1.4% to about 1.6%, about 1.5% to about 1.7%, about 1.6% to about 1.8%, or about 1.7% to about 1.9% w/w of the composition. For example, about 0.25%, about 0.3%, about 0.35%, about 0.4%, about 0.45%, about 0.5%, about 0.55%, about 0.6%, about 0.65%, about 0.7%, about 0.72%, about 0.74%, about 0.76%, about 0.78%, about 0.8%, about 0.81%, about 0.82%, about 0.83%, about 0.84%, about 0.85%, about 0.86%, about 0.87%, about 0.88%, about 0.89%, about 0.9%, about 0.91%, about 0.92%, about 0.93%, about 0.94%, about 0.96%, about 0.98%, about 1%, about 1.05%, about 1.1%, about 1.15%, about 1.2%, about 1.25%, about 1.3%, about 1.35%, about 1.4%, about 1.5%, about 1.6%, about 1.7%, about 1.8%, about 1.9%, or about 2% w/w of the composition.

In some embodiments, the one or more amines comprise triethanolamine. In some embodiments, the one or more amines is triethanolamine. In some embodiments, triethanolamine is present in an amount of about 0.01% to about 2% w/w of the composition. For example, about 0.01%, to about 0.05%, about 0.01%, to about 0.1%, about 0.01%, to about 0.25%, about 0.01%, to about 0.5%, about 0.01%, to about 0.75%, about 0.01% to about 1%, about 0.01% to about 1.25%, about 0.01% to about 1.5%, about 0.01% to about 1.75%, about 0.01% to about 2%, about 1.75% to about 2%, about 1.5% to about 2%, about 1.25% to about 2%, about 1% to about 2%, about 0.75% to about 2%, about 0.5% to about 2%, about 0.25% to about 2%, about 0.1% to about 2%, about 0.05% to about 2% w/w of the composition. In some embodiments, the triethanolamine is present in an amount of 0.25% to about 1.25%, about 0.2% to about 0.3%, about 0.25% to about 0.35%, about 0.3% to about 0.4%, about 0.35% to about 0.45%, about 0.4% to about 0.5%, about 0.45% to about 0.55%, about 0.5% to about 0.6%, about 0.55% to about 0.65%, about 0.6% to about 0.7%, about 0.65% to about 0.75%, about 0.7% to about 0.8%, about 0.75% to about 0.85%, about 0.8% to about 0.9%, about 0.85% to about 0.95%, about 0.9% to about 1%, about 0.95% to about 1.05%, about 1% to about 1.1%, about 1.05% to about 1.15%, about 1.1% to about 1.2%, about 1.15% to about 1.25%, about 1.2% to about 1.3%, about 1.25% to about 1.35%, about 1.3% to about 1.4%, about 1.35% to about 1.45%, about 1.4% to about 1.6%, about 1.5% to about 1.7%, about 1.6% to about 1.8%, or about 1.7% to about 1.9% w/w of the composition. For example, about 0.25%, about 0.3%, about 0.35%, about 0.4%, about 0.45%, about 0.5%, about 0.55%, about 0.6%, about 0.65%, about 0.7%, about 0.72%, about 0.74%, about 0.76%, about 0.78%, about 0.8%, about 0.81%, about 0.82%, about 0.83%, about 0.84%, about 0.85%, about 0.86%, about 0.87%, about 0.88%, about 0.89%, about 0.9%, about 0.91%, about 0.92%, about 0.93%, about 0.94%, about 0.96%, about 0.98%, about 1%, about 1.05%, about 1.1%, about 1.15%, about 1.2%, about 1.25%, about 1.3%, about 1.35%, about 1.4%, about 1.5%, about 1.6%, about 1.7%, about 1.8%, about 1.9%, or about 2% w/w of the composition.

In some embodiments, the one or more amines comprise panthenol. In some embodiments, the one or more amines is panthenol. In some embodiments, panthenol is present in an amount of about 0.01% to about 2% w/w of the composition. For example, about 0.01%, to about 0.05%, about 0.01%, to about 0.1%, about 0.01%, to about 0.25%, about 0.01%, to about 0.5%, about 0.01%, to about 0.75%, about 0.01% to about 1%, about 0.01% to about 1.25%, about 0.01% to about 1.5%, about 0.01% to about 1.75%, about 0.01% to about 2%, about 1.75% to about 2%, about 1.5% to about 2%, about 1.25% to about 2%, about 1% to about 2%, about 0.75% to about 2%, about 0.5% to about 2%, about 0.25% to about 2%, about 0.1% to about 2%, about 0.05% to about 2% w/w of the composition. In some embodiments, the panthenol is present in an amount of 0.25% to about 1.25%, about 0.2% to about 0.3%, about 0.25% to about 0.35%, about 0.3% to about 0.4%, about 0.35% to about 0.45%, about 0.4% to about 0.5%, about 0.45% to about 0.55%, about 0.5% to about 0.6%, about 0.55% to about 0.65%, about 0.6% to about 0.7%, about 0.65% to about 0.75%, about 0.7% to about 0.8%, about 0.75% to about 0.85%, about 0.8% to about 0.9%, about 0.85% to about 0.95%, about 0.9% to about 1%, about 0.95% to about 1.05%, about 1% to about 1.1%, about 1.05% to about 1.15%, about 1.1% to about 1.2%, about 1.15% to about 1.25%, about 1.2% to about 1.3%, about 1.25% to about 1.35%, about 1.3% to about 1.4%, about 1.35% to about 1.45%, about 1.4% to about 1.6%, about 1.5% to about 1.7%, about 1.6% to about 1.8%, or about 1.7% to about 1.9% w/w of the composition. For example, about 0.25%, about 0.3%, about 0.35%, about 0.4%, about 0.45%, about 0.5%, about 0.55%, about 0.6%, about 0.65%, about 0.7%, about 0.72%, about 0.74%, about 0.76%, about 0.78%, about 0.8%, about 0.81%, about 0.82%, about 0.83%, about 0.84%, about 0.85%, about 0.86%, about 0.87%, about 0.88%, about 0.89%, about 0.9%, about 0.91%, about 0.92%, about 0.93%, about 0.94%, about 0.96%, about 0.98%, about 1%, about 1.05%, about 1.1%, about 1.15%, about 1.2%, about 1.25%, about 1.3%, about 1.35%, about 1.4%, about 1.5%, about 1.6%, about 1.7%, about 1.8%, about 1.9%, or about 2% w/w of the composition.

In some embodiments, an aftercare composition can further comprise one or more preservatives. In some embodiments, the one or more preservatives are present in an amount of about 0.01% to about 2% w/w of the composition. For example, about 0.01%, to about 0.05%, about 0.01%, to about 0.1%, about 0.01%, to about 0.25%, about 0.01%, to about 0.5%, about 0.01%, to about 0.75%, about 0.01% to about 1%, about 0.01% to about 1.25%, about 0.01% to about 1.5%, about 0.01% to about 1.75%, about 0.01% to about 2%, about 1.75% to about 2%, about 1.5% to about 2%, about 1.25% to about 2%, about 1% to about 2%, about 0.75% to about 2%, about 0.5% to about 2%, about 0.25% to about 2%, about 0.1% to about 2%, about 0.05% to about 2% w/w of the composition. In some embodiments, the one or more preservatives are present in an amount of 0.25% to about 0.75%, about 0.2% to about 0.3%, about 0.25% to about 0.35%, about 0.3% to about 0.4%, about 0.35% to about 0.45%, about 0.4% to about 0.5%, about 0.45% to about 0.55%, about 0.5% to about 0.6%, about 0.55% to about 0.65%, about 0.6% to about 0.7%, about 0.65% to about 0.75%, about 0.7% to about 0.8%, about 0.75% to about 0.85%, about 0.8% to about 0.9%, about 0.85% to about 0.95%, about 0.9% to about 1%, about 0.95% to about 1.05%, about 1% to about 1.1%, about 1.05% to about 1.15%, about 1.1% to about 1.2%, about 1.15% to about 1.25%, about 1.2% to about 1.3%, about 1.25% to about 1.35%, about 1.3% to about 1.4%, about 1.35% to about 1.45%, about 1.4% to about 1.6%, about 1.5% to about 1.7%, about 1.6% to about 1.8%, or about 1.7% to about 1.9% w/w of the composition. For example, about 0.25%, about 0.3%, about 0.35%, about 0.4%, about 0.45%, about 0.5%, about 0.55%, about 0.6%, about 0.65%, about 0.7%, about 0.72%, about 0.74%, about 0.76%, about 0.78%, about 0.8%, about 0.81%, about 0.82%, about 0.83%, about 0.84%, about 0.85%, about 0.86%, about 0.87%, about 0.88%, about 0.89%, about 0.9%, about 0.91%, about 0.92%, about 0.93%, about 0.94%, about 0.96%, about 0.98%, about 1%, about 1.05%, about 1.1%, about 1.15%, about 1.2%, about 1.25%, about 1.3%, about 1.35%, about 1.4%, about 1.5%, about 1.6%, about 1.7%, about 1.8%, about 1.9%, or about 2% w/w of the composition.

In some embodiments, the one or more preservatives are selected form the group consisting of: ascorbic acid, an ascorbate, a palmitate, citric acid, a benzoate, a benzoic acid, a propionate, propionic acid, a sorbate, sorbic acid, a salicylic acid, a salicylate, hexa-2,4-dienoic acid, a hexa-2,4-dienoate, formaldehyde, a formaldehyde releaser, formic acid and its salts, 3-acetyl-6-methylpyran-2,4-(3H)-dione and its salts, 3,3′-dibromo-4,4′-hexamethylenedioxydibenzamidine and its salts, thiomersal, phenylmercuric salts, undec-10-enoic acid and its salts, 1,3-bis (2-ethylhexyl) hexahydro-5-methyl-5-pyrimidine, 5-bromo-5-nitro-1,3-dioxane, bronopol, 2,4-dichlorobenzyl alcohol, 1-(4-chlorophenyl)-3-(3,4-dichlorophenyl) urea, chlorocresol, chloroxylenol, 5-chloro-2-(2,4-dichlorophenoxy) phenol, N,N″-methylenebis [N′-[3-(hydroxymethyl)-2,5-dioxoimidazolidin-4-yl]urea], polyaminopropyl biguanide, methenamine, quaternium-15, climbazole, DMDM hydantoin, benzyl alcohol, 1-Hydroxy-4-methyl-6-(2,4,4-trimethylpentyl)-2-pyridon, piroctone olamine, bromochlorophene, o-cymen-5-ol, chlorophene, chloroacetaminde, methylchloroisothiazolinone, methylisothiazolinone, phenoxyisopropanol, chlorhexidine, chlorhexidine diacetate, chlorhexidine digluconate, chlorhexidine dihydrochloride, dimethyl oxazolidine, behentrimonium chloride, cetri-monium bromide, cetrimonium chloride, laurtrimonium bromide, laurtrimonium chloride, steartrimonium bromide, steartrimonium chloride, diazolidinyl urea, hexamidine, hexamidine diisethionate, hexamidine paraben, glutaral, 7-ethylbicyclooxazolidine, chlorphenesin, sodium hydroxymethylglycinate, silver chloride, benzethonium chloride, benzalkonium chloride, benza-lkonium bromide, benzalkonium saccharinate, benzylhemiformal, iodopropynyl butylcarbamate, biphenyl-2-ol and its salts, pyrithionine zinc, an erythorbate, a nitrite, ethylenediaminetetraacetic acid (EDTA), butylated hydroxyanisole (BHA), butylated hydroxytoluene (BHT), capryllic acid, dilauryl thiodipropionate, erythorbic acid, gum guaiac, methylparaben, a sulfite, a bisulfite, a metabisulfite, propyl gallatepy, propylparaben, stannous chloride, sulfur dioxide, thiodipropionic acid, an isothiazoline, a paraben, phenoxyethanol, ethylhexylglycerin, a glycols, and a tocopherol.

In some embodiments, an aftercare composition comprises:

one or more volatile solvents present in an amount of about 30% to about 40% w/w of the composition;

one or more humectants present in an amount of about 20% to about 30% w/w of the composition; and

one or more occlusive agents present in an amount of about 0.2% to about 0.4% w/w of the composition.

In some embodiments, the one or more volatile solvents comprise water and ethanol. In some embodiments, the one or more humectants comprise glycerin. In some embodiments, the one or more occlusive agents comprise an acrylate/C10-30alkyl acrylate crosspolymer.

In some embodiments, the composition further includes one or more emollients. In some embodiments, wherein at least one of the one or more emollients also function as an occlusive agent, the at least one emollient that also functions as an occlusive agent is a combination of dimethicone, trisiloxane, ceteth-10, and laureth-4 (e.g., Dowsil 7-3110 Volatile Fluid HIP Emulsion). In some embodiments, the at least one emollient that also functions as an occlusive agent is present in an amount of about 35% to about 40% w/w of the composition.

In some embodiments, the aftercare composition further includes one or more amines. In some embodiments, the one or more amines are present in an amount of about 0.25% to about 1.25% w/w of the composition.

In some embodiments, the aftercare composition further includes one or more preservatives. In some embodiments, the one or more preservatives are present in an amount of about 0.25% to about 0.75% w/w of the composition.

In some embodiments, an aftercare composition comprises:

water present in an amount of about 30% to about 40% w/w of the composition;

glycerin present in an amount of about 20% to about 30% w/w of the composition; and

an acrylate/C10-30alkyl acrylate crosspolymer present in an amount of about 0.2% to about 0.4% w/w of the composition.

In some embodiments, the composition further includes one or more emollients. In some embodiments, wherein at least one of the one or more emollients also function as an occlusive agent, the at least one emollient that also functions as an occlusive agent is a combination of dimethicone, trisiloxane, ceteth-10, and laureth-4 (e.g., Dowsil 7-3110 Volatile Fluid HIP Emulsion). In some embodiments, the at least one emollient that also functions as an occlusive agent is present in an amount of about 35% to about 40% w/w of the composition.

In some embodiments, the aftercare composition further includes one or more amines. In some embodiments, the one or more amines are selected from the group consisting of: triethanolamine, L-arginine, and panthenol. In some embodiments, the one or more amines are present in an amount of about 0.25% to about 1.25% w/w of the composition.

In some embodiments, an aftercare composition comprises:

one or more volatile solvents present in an amount of about 65% to about 75% w/w of the composition;

one or more humectants present in an amount of about 20% to about 30% w/w of the composition; and

one or more occlusive agents present in an amount of about 0.2% to about 0.4% w/w of the composition.

In some embodiments, the one or more volatile solvents comprise water and ethanol. In some embodiments, the one or more humectants comprise glycerin. In some embodiments, the one or more occlusive agents comprise an acrylate/C10-30alkyl acrylate crosspolymer.

In some embodiments, the aftercare composition further includes one or more amines. In some embodiments, the one or more amines are present in an amount of about 0.25% to about 1.25% w/w of the composition.

In some embodiments, the aftercare composition further includes one or more preservatives. In some embodiments, the one or more preservatives are present in an amount of about 0.25% to about 0.75% w/w of the composition.

In some embodiments, an aftercare composition comprises:

water present in an amount of about 60% to about 75% w/w of the composition;

glycerin present in an amount of about 20% to about 30% w/w of the composition; and

an acrylate/C10-30alkyl acrylate crosspolymer present in an amount of about 0.2% to about 0.4% w/w of the composition.

In some embodiments, the composition further comprises ethanol. In some embodiments, the water is present in an amount of about 60% to about 70% w/w of the composition and the ethanol is present in an amount of about 5% to about 15% w/w of the composition.

In some embodiments, the aftercare composition further includes one or more amines. In some embodiments, the one or more amines are selected from the group consisting of: triethanolamine, L-arginine, and panthenol. In some embodiments, the one or more amines are present in an amount of about 0.25% to about 1.25% w/w of the composition.

In some embodiments, an aftercare composition can be configured to have a pH of about 4 to about 7. For example, about 4 to about 5, about 4.5 to about 5.5, about 5 to about 6, about 5.5 to about 6.5, or about 6 to about 7. In some embodiments, the aftercare composition can be configured to have a pH of a pH of about 4, about 4.25, about 4.5, about 4.75, about 5, about 5.25, about 5.5, about 5.75, about 6, about 6.25, about 6.5, about 6.75, or about 7.

In some embodiments, an aftercare composition can have a pH of about 4 to about 7. For example, about 4 to about 5, about 4.5 to about 5.5, about 5 to about 6, about 5.5 to about 6.5, or about 6 to about 7. In some embodiments, the aftercare composition can have a pH of a pH of about 4, about 4.25, about 4.5, about 4.75, about 5, about 5.25, about 5.5, about 5.75, about 6, about 6.25, about 6.5, about 6.75, or about 7.

In some embodiments, an aftercare composition as provided herein can further include one or more semi-volatile semi-permanent colorant solubilizers as described herein.

In some embodiments, an aftercare composition as provided herein can be formulated as a serum, a cream, a mousse, a semisolid stick, a spray, a moisturized patch, or a roll-on.

In some embodiments, a suitable aftercare composition can include an unscented lotion. For example, an unscented lotion can have the following ingredients: water, grapeseed oil (Vitis vinifera), complete emulsifying wax B, glycerin (e.g., vegetable glycerin), vitamin E oil, and a combination of benzyl alcohol, salicylic acid, glycerin, and sorbic acid (e.g., GEOGARD® ECT).

In some embodiments, aftercare compositions are applied to a subject after the application of a semi-permanent tattoo to the subject. For example, about 5 minutes, about 15 minutes, about 30 minutes, about 1 hour, or about 2 hours after the application of a semi-permanent tattoo. In some embodiments, aftercare compositions are applied to the subject daily. For example, one, two, three, four, five, or more times per day. In some embodiments, aftercare compositions are applied to the subject every other day or every three days. In some embodiments, aftercare compositions are applied to the subject daily for the duration of the semi-permanent tattoo.

In some embodiments, aftercare compositions are applied to the subject after bathing. In some embodiments, aftercare compositions are applied to the subject after washing the area of skin where the semi-permanent tattoo was applied on the subject. In some embodiments, aftercare compositions are applied to the subject after the subject has exercised.

Also provided herein is an aftercare article moistened with the aftercare composition of any of the above embodiments. In some embodiments, the moistened article can be a towelette, a pad, a woven, a nonwoven, or the like. In some embodiments, the moistened article can be contained within a sealed package in order to maintain the moist nature of the article.

Also provided herein are methods for prolonging the duration of a semi-permanent tattoo in skin comprising applying an aftercare composition of any of the above embodiments. In some embodiments, the method for prolonging the duration of a semi-permanent tattoo in skin comprises prolonging the duration of the semi-permanent tattoo in the stratum corneum. In some embodiments, prolonging the duration of a semi-permanent tattoo comprises decreasing the loss of intensity of the semi-permanent tattoo after it has been applied to skin compared to the loss of intensity of a semi-permanent tattoo not exposed to an aftercare composition.

In some embodiments, the semi-permanent tattoo is applied to the skin of a subject prior to application of an aftercare composition. In some embodiments, the aftercare composition is applied to the skin of a subject after the application of a semi-permanent tattoo to the subject. For example, after about 5 minutes, about 15 minutes, about 30 minutes, about 1 hour, or about 2 hours after the application of a semi-permanent tattoo. In some embodiments, the aftercare composition is applied to the skin of a subject immediately after the semi-permanent tattoo was applied to the skin of the subject. In some embodiments, aftercare compositions are applied to the skin of a subject daily. For example, one, two, three, four, five, or more times per day. In some embodiments, aftercare compositions are applied to the skin of a subject every other day, every three days, or weekly. In some embodiments, aftercare compositions are applied to the skin of a subject daily for the duration of the semi-permanent tattoo on the subject. In some embodiments, the aftercare composition is applied over the area of skin where the semi-permanent tattoo was applied on the subject.

Also provided herein are methods for applying a temporary tattoo ink to an article.

In some embodiments, the ink composition (e.g., the ink composition 60 of FIG. 1) is applied to the first surface 121 of the first adhesive layer 120 as shown in FIG. 2A.

In some embodiments, the ink composition 160 is applied by inkjet printing.

In some embodiments, the ink composition (e.g., the ink composition 60 of FIG. 1) is applied to the first surface of the first adhesive layer 220 described in connection with FIG. 3.

In some embodiments, the ink composition 260 is applied by inkjet printing.

In some embodiments, the ink cartridges of the inkjet printer used to print an ink composition as described above onto an adhesive applicator article as described above can be loaded with two different ink compositions. For example, when two ink cartridges are used in the printer, one ink cartridge, e.g., the dark cyan cartridge, may be loaded with an ink composition having a greater concentration of a semi-permanent colorant and/or temporary colorant than the ink composition loaded into the second ink cartridge, e.g., the light cyan cartridge.

Also provided herein is a kit comprising the adhesive applicator article of any one of the above adhesive applicator article embodiments. In some embodiments, the adhesive applicator article is an adhesive applicator article with a heat pad. In some embodiments, the kit further comprises a heat pad (e.g., separate from the adhesive applicator article). In some embodiments, the adhesive applicator article is an adhesive applicator article with an occlusive patch. In some embodiments, the kit further comprises a skin priming composition of any of the above embodiments. In some embodiments, the kit further comprises a skin priming composition of any of the above embodiments. In some embodiments of the kit, a priming article, e.g., a towelette, may be provided with the skin priming composition. In some embodiments, the skin priming composition can be provided in the kit on a priming article, e.g., a towelette, premoistened with the skin priming composition. In some embodiments, the kit further includes instructions.

Also provided herein is a kit comprising the adhesive applicator article of any one of the above adhesive applicator article embodiments and a skin priming composition of any of the above embodiments. In some embodiments, the adhesive applicator article is an adhesive applicator article with a heat pad. In some embodiments, the kit further comprises a heat pad (e.g., separate from the adhesive applicator article). In some embodiments, the adhesive applicator article is an adhesive applicator article with an occlusive patch. In some embodiments, the kit further includes a towelette. In some embodiments, the towelette is moistened with the skin priming composition. In some embodiments of the kit, a priming article, e.g., a towelette, may be provided with the skin priming composition. In some embodiments, the skin priming composition may be provided in the kit on a priming article, e.g., a towelette, premoistened with the skin priming composition. In some embodiments, the kit further includes instructions. In some embodiments, the kit further includes an aftercare composition as described herein.

Also provided herein is a kit comprising the adhesive applicator article of any one of the above adhesive applicator article embodiments; a skin priming composition of any of the above embodiments; and a heat pad. In some embodiments of the kit, a priming article, e.g., a towelette, may be provided with the skin priming composition. In some embodiments, the skin priming composition may be provided in the kit on a priming article, e.g., a towelette, premoistened with the skin priming composition. In some embodiments, the kit further includes instructions. In some embodiments, the kit further includes an aftercare composition as described herein.

Additional extra ink (temporary and/or semi-permanent colorant) may be added to each above kit to allow e.g., correction, personalization, recoloring, etc. of the semi-permanent tattoo by freehand application. In some embodiments, the extra ink can have a composition as described above, or as described in U.S. Pat. No. 10,143,641, which is incorporated by reference herein in its entirety.

A kit comprising the inkjet composition and/or an inkjet printing cartridge containing the inkjet composition, and an applicator article without the composition printed on it, for printing a custom design using an inkjet printer, or freehand drawing a design onto the applicator prior to application by a user.

### EXAMPLES

The invention will be described in greater detail by way of specific examples. The following examples are offered for illustrative purposes, and to exemplify the topical compositions and methods described herein and are not intended to limit the invention in any manner. Many variations will suggest themselves and are within the full-intended scope. Those of skill in the art will readily recognize a variety of non-critical parameters that can be changed or modified to yield essentially the same results.

### Example 1. Ink Compositions

A. Preparation of Ink Compositions

Fifteen ink compositions were prepared as shown in Table 1.

TABLE 1
Ink Compositions.

| | Composition (% w/w) | | | | | | |
|---|---|---|---|---|---|---|---|
| | | | diethylene glycol | | | | |
| Composition | | | monoethyl ether | | PEG | | |
| Number | Water | Ethanol | (e.g., TRANSCUTOL ®) | Genipin | 400 | Trehalose | HPMC |
| 1 | 50 | 25 | 25 | — | — | — | — |
| 2 | 47 | 27 | 17 | 9 | — | — | — |
| 3 | 47 | 22 | 22 | 9 | 1 | — | — |
| 4 | 46 | 21 | 21 | 9 | 2 | — | — |
| 5 | 46 | 21 | 21 | 9 | 3 | — | — |
| 6 | 47 | 22 | 22 | 9 | — | 1 | — |
| 7 | 46 | 21 | 21 | 9 | — | 2 | — |
| 8 | 46 | 21 | 21 | 9 | — | 3 | — |
| 9 | 46 | 21 | 21 | 9 | 1 | 2 | — |
| 10 | 46 | 21 | 21 | 9 | 2 | 2 | — |
| 11 | 45 | 20 | 20 | 9 | 3 | 2 | — |
| 12 | 46 | 21 | 21 | 9 | 3 | — | 1 |
| 13 | 46 | 21 | 21 | 9 | 3 | — | 1 |
| 14 | 46 | 21 | 21 | 9 | 4 | — | 1 |
| 15 | 46 | 21 | 21 | 9 | 2 | — | 1 |

For the ink compositions in Table 2, the solids, (genipin, trehalose, and HPMC) were weighed out. The liquids (water, buffer solution, ethanol, and diethylene glycol monoethyl ether) were then weighed out together in a 250 mL beaker. The liquids were then placed on a mechanical mixer and mixed at a speed of 400 RPM. The beaker was also covered with PVC plastic wrap to prevent evaporation of the volatile solutions. Trehalose was introduced into the mixture in aliquots and allowed to mix thoroughly until all of it was dissolved. The beaker was then removed from the mixer and placed onto the weighing machine and the surfactants/preservatives were introduced. The preservatives and surfactants in this composition refer to EUXYL® PE9010 (e.g., 9:1 phenoxyethanol:ethylhexylglycerin) and PEG 400, respectively. The resultant mixture is then mixed using the mechanical mixer at 400 RPM until a uniform solution is achieved. While mixing, the genipin solids is added to the resultant solution and allowed to mix thoroughly until a uniform solution is achieved. The resultant mixture is put onto a vacuum filter with 1.2 micron pore size and filtered into a sterilized glass jar.

TABLE 2
Ink Compositions.

| | Composition (% w/w) | | | | | |
|---|---|---|---|---|---|---|
| | | | diethylene glycol | | | |
| Composition | Buffer | | monoethyl ether | | EUXYL ® | |
| Number | Solution | Ethanol | (e.g., TRANSCUTOL ®) | Genipin | PE9010* | Trehalose |
| 19 | 46 | 28 | 13 | 7 | 1 | 5 |
| 20 | 45.8 | 28.4 | 13.3 | 7 | 0.5 | 5 |
| 21 | 52.50 | 20.25 | 12.75 | 7 | 1 | 6.5 |
| 22 | 52.50 | 20.375 | 12.875 | 7 | 0.750 | 6.5 |
| 23 | 46 | 21 | 21 | 9 | 1 | 2 |

*EUXYL ® PE9010 is about 9:1 phenoxyethanol:ethylhexylglycerin

Additional ink compositions were prepared as shown in Tables 3 and 4.

TABLE 3
Ink Compositions

| | Composition (% w/w) | | | | | | |
|---|---|---|---|---|---|---|---|
| | | | diethylene glycol | | | | Acid Blue 9 |
| Composition | | | monoethyl ether | | PEG | | (e.g, FD&C |
| Number | Water | Ethanol | (e.g., TRANSCUTOL ®) | Genipin | 400 | Trehalose | Blue No. 1) |
| 9.1 | 45.59 | 20.97 | 20.97 | 8.99 | 1 | 2 | 0.2 |

TABLE 4
Ink Compositions

| | Composition (% w/w) | | | | | | |
|---|---|---|---|---|---|---|---|
| | | | diethylene glycol | | | | Acid Blue 9 |
| Composition | | | monoethyl ether | | PEG | | (e.g, FD&C |
| Number | Water | Ethanol | (e.g., TRANSCUTOL ®) | Genipin | 400 | Trehalose | Blue No. 1) |
| 9.1 | 47.46 | 22.48 | 22.48 | 4.5 | 1 | 2 | 0.1 |

B. Characterization Methods

Characterization Method #1: In Vitro Film Dissolution

Dissolution Solution Preparation

To prepare the dissolution solution, 54 mL of Fisher Scientific pH 4.1 buffer solution (simulating eccrine sweat pH) are added to a 200 mL graduated cylinder followed by 0.469 g of L-arginine. The solution is agitated until the L-arginine dissolves completely. Subsequently, 6 mL of isopropanol are added to the 200 mL beaker. The dissolution solution is stored in airtight 25 mL scintillation vials.

Sample and Test Site Preparation

3 mL of an ink composition as described in Tables 1 or 2 is dispersed onto a 9 cm petri dish with uniform coating. The sample is placed in a dehydrator at 150° F. for 2 hours to allow a film to form. The film's physical characteristics such as age from time of film cast, genipin crystal morphology, color and transparency, uniformity, and thickness are recorded. All samples that are tested in the same batch are kept at the same room conditions, as crystal morphology can vary depending on conditions.

### Experiment

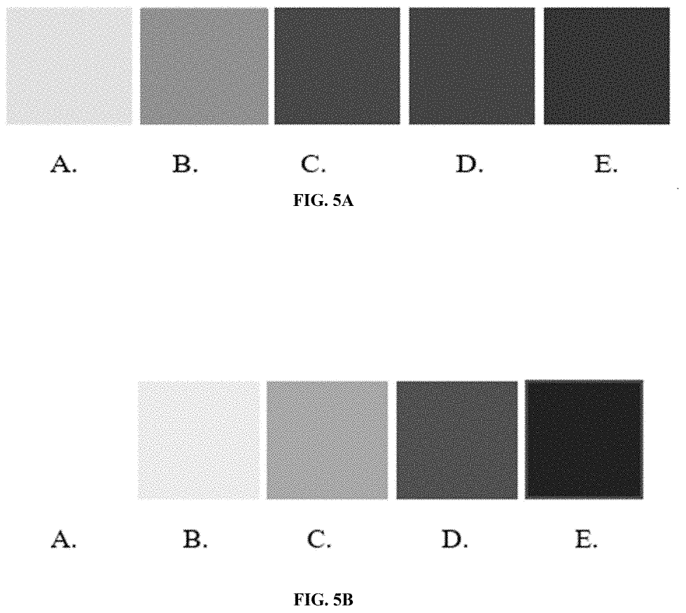

20 mL of the dissolution solution is added to each petri dish containing the cast film and heated to 40° C. The time elapsed to achieve the colors corresponding to the color scales in FIG. 5 over the course of 60 seconds are recorded. The total time required to dissolve the film into the dissolution solution is also recorded.

Characterization Method #2: In Vitro Printed Substrate Total Content Analysis

Genipin dissolution from dried printed adhesive substrates was measured utilizing high performance liquid chromatography (HPLC). Printed samples, in which Compositions 3, 4, and 9 were deposited on the 2×2 cm adhesive substrate, were placed in a 9 cm diameter petri dish and immersed in 10 mL of methanol for 30 minutes. The adhesive was then removed from the petri dish and rinsed for 30 seconds each in a vial containing 25 mL of methanol. The adhesive was then removed and immersed in a 25 mL of methanol for 24 hour extraction. The extraction solutions were evaporated to recover the extracted genipin. Total genipin content was then determined via HPLC.

Characterization Method #3: Quantitative Analysis of Genipin Dissolution from Printed Adhesive Substrate

The dissolution buffer is prepared by adding 54 mL of Fisher Scientific pH 4.1 buffer solution to a 200 mL beaker followed by 6 mL of isopropanol.

Test samples comprising 2 inches by 2 inches of printed ink area using 5 printer passes are printed onto the adhesive substrate. The printed samples are stored for designated time periods to determine genipin release stability.

Petri dishes are heated to 40° C. Each test sample is adhered to a petri dish (one test sample per petri dish) using double-sided tape. 20 mL of the dissolution buffer are introduced to the covered petri dishes to immerse the test samples for 60 minutes. The dissolution buffer is removed using a syringe and transferred to a scintillation vial for HPLC analysis.

Characterization Method #4: Skin Ink Density Profile Quantitation

Test Protocol

Test semi-permanent tattoos are applied to study participants meeting the following criteria: no previous semi-permanent tattoo application in the past 2 weeks at the test site, no topical product use in the last 24 hours at the test site, and no other marks on skin interfering with digital analysis, as determined by study staff. Two different test sites are selected per study participant to apply two predetermined test articles. Some of the test sites are located on the upper volar forearm and comprised four square semi-permanent tattoos as shown in FIG. 6. Other test sites include the inner upper arm. Each test site is gently wiped with an ethanol wipe and dabbed dry with a KIMWIPE™. The freshly printed semi-permanent tattoo adhesive are affixed to the test site. After 1 hour, the applicator is removed.

For liquid compositions, a designated study staff applies a target 0.05 g of the test article to each of the four squares.

Ink Density Data Collection

Photography

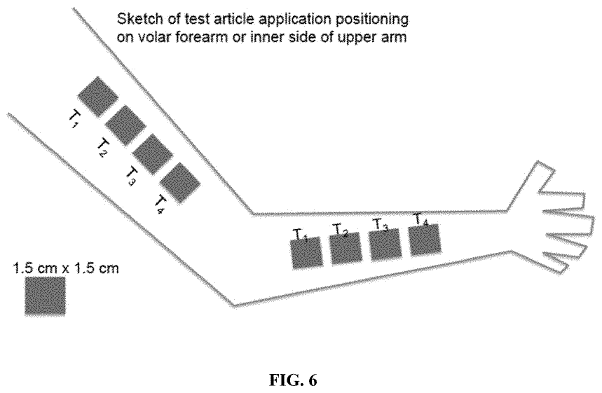

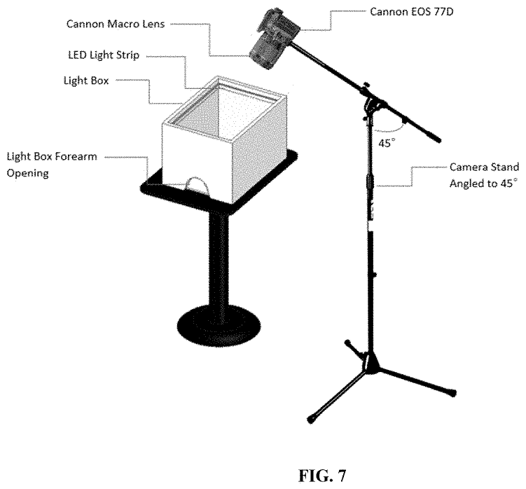

Using the standardized photography suite as shown in FIG. 7, photographs of the test site (including each of the four inked squares) are taken at each time point prior to each tape-stripping of the inked square. A final photograph is taken following the last tape strip. A total of 21 photographs for each inked square are taken at each time point, yielding 64 total photos for the entire test site that are evaluated utilizing an Image J process. The Canon EOS77D and with an ultrasonic Macro lens is used with the standardized photography suite to capture the semi-permanent tattooed skin sites.

Visual Grading

A designated evaluator not in the study grades the ink intensity (prior to the tape stripping at following the removal of each tape strip) on a scale of 0-4, using the guidelines in Table 5.

TABLE 5
Ink Intensity Scale

| | |
|---|---|
| 0 | no visible ink |
| 1 | faintly visible ink |
| 2 | moderately visible ink |
| 3 | target ink density |
| 4 | superior to target ink density |

The study participant evaluates the test site at each time-point before the site is tape stripped and following the removal of each tape strip, using the guidelines in Table 6.

TABLE 6
Performance Evaluation

| | |
|---|---|
| 0 | no visible ink |
| 1 | visible ink, unacceptable performance |
| 2 | visible ink, acceptable performance |
| 3 | visible ink, desired performance |

Tape Strip

At each predetermined time-point, a designated member of the study staff removes a total of 20 tape strips from each inked square, following the guidelines of Lademann et al., Eur. J. Pharm. Biopharm. 72:317-323, 2009, which is incorporated by reference herein in its entirety. The designated member of the study staff uses sterilized tweezers to peel a precut 1.5 inch×1.5 inch, single-sided adhesive square from the sheet and applies it to the predetermined test site. A roller is used to apply even pressure to the adhesive to ensure that it is in contact with the skin. The adhesive is removed ensuring that the tape was pulled in the direction of hair growth and low to the skin at a constant speed. The adhesive squares are then placed on glass slides and marked with the formulation, the time point, and the tape strip number. The glass slides with the adhesive squares are retained for future analyses.

The data are analyzed as follows: 1) The ink density as a function of tape strip number is plotted for each time point; 2) Photographs re processed with Image J utilizing two macros: Percent Green and Color Histogram; 3) The change in statistical pixel intensity distribution is analyzed to follow the shift towards the darker pixel intensity. The change in statistical pixel intensity distribution is used to determine darkness and gauge ink development (or genipin polymerization and/or oligomerization). Using the color histogram, the standard deviation and mode of the pixel intensity for blue and green are plotted for each time point to assess the amount of ink development.

Characterization Method #5: Stability of Printed Ink Compositions

Adhesive substrates with printed ink were placed at 25° C. and 60% room humidity (normal condition) and at 40° C. and 75% room humidity (accelerated condition) for 2 weeks. At the 2 week time point, the samples were removed from the stability chambers and submitted for dissolution testing and content analysis.

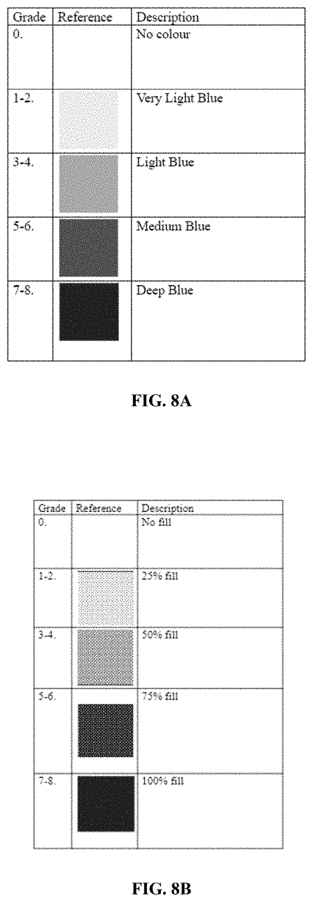

The semi-permanent tattoos were applied and given 36 hours to develop. The skin transfer results were visually graded based on the 0-8 ink intensity scale and ink density scale shown in FIG. 8A and the 1-8 image quality scale shown in FIG. 8B.

Characterization Method #6: Viscosity and Surface Tension of Compositions

A Brookfield Ametek DVE Viscometer, model no. DVEELVTJO was used to determine the viscosity of all measured compositions.

The tensiometer used to obtain surface tension and contact angle measurements was a Droplet Labs Drop-O-Meter Automatic or Manual tensiometer and data was processed using the Sessile data processing application.

C. Ink Composition Characterization Results

In Vitro Printed Substrate Total Content Analysis

Table 7 shows the formulation number and the corresponding adhesives used for extraction and determination of genipin content via HPLC.

TABLE 7

| Formulation/ | Composition | |
|---|---|---|
| Adhesive Identifier | Number | Adhesive |
| I | 9 | TM8750 |
| II | 3 | DM8007 |
| III | 4 | TM8750 |
| IV | 9 | 3M1509 |
| V | 4 | DM8007 |
| VI | 4 | 3M1509 |
| VII | 3 | 3M1509 |
| VIII | 9 | DM8007 |

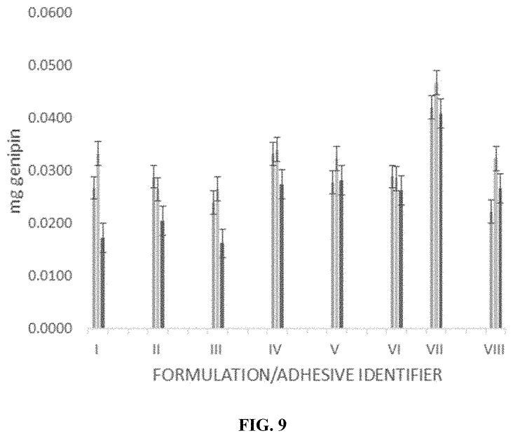

FIG. 9 shows the amount of genipin extracted from each dried printed adhesive substrate.

Stability of Printed Ink Compositions

The skin transfer grading results for compositions from Table 1 and adhesive substrates put on stability are outlined in Table 8 and Table 9 for normal and accelerated conditions, respectively.

TABLE 8
Visual Skin Transfer Grading at Normal
Conditions (25 deg. C., 60% Humidity)

| | DM8007 | 3M1509 | TM8750 |
|---|---|---|---|
| Composition 3 | Darkness | Darkness | |
| | Ranking: 3 | Rankin: 7 | |
| | Image Integrity | Image Integrity | |
| | Ranking: 3 | Ranking: 7 | |
| Composition 4 | Darkness | Darkness | |
| | Ranking: 5 | Ranking: 3 | |
| | Image Integrity | Image Integrity | |
| | Ranking: 4 | Ranking: 6 | |
| Composition 9 | Darkness | Darkness | Darkness |
| | Ranking: 4 | Ranking: 8 | Ranking: 8 |
| | Image Integrity | Image Integrity | Image Integrity |
| | Ranking: 4 | Ranking: 7 | Ranking: 8 |

TABLE 9
Visual Skin Transfer Grading at Accelerated
Conditions (40 deg. C., 70% Humidity)

| | DM8007 | 3M1509 | TM8750 |
|---|---|---|---|
| Composition 3 | Darkness | Darkness | |
| | Ranking: 6 | Ranking: 5 | |
| | Image Integrity | Image Integrity | |
| | Ranking: 2 | Ranking: 3 | |
| Composition 4 | Darkness | Darkness | |
| | Ranking: 5 | Ranking: 7 | |
| | Image Integrity | Image Integrity | |
| | Ranking: 2 | Ranking: 7 | |
| Composition 9 | Darkness | Darkness | Darkness |
| | Ranking: 5 | Ranking: 6 | Ranking: 5 |
| | Image Integrity | Image Integrity | Image Integrity |
| | Ranking: 1 | Ranking: 5 | Ranking: 8 |

Surface Tension and Viscosities

Table 10 shows the surface tension and viscosity measurements for the compositions described in Table 1.

TABLE 10
Surface tension and viscosity measurements

| Composition | Viscosity | Surface Tension |
|---|---|---|
| Number | (Centipoise) | (average) dynes/cm |
| 1 | 3.54 | 30.084 |
| 2 | 3.86 | 29.49 |
| 3 | 4.28 | 32.004 |
| 4 | 4.38 | 33.021 |
| 5 | 4.52 | 28.973 |
| 6 | 4.4 | 28.481 |
| 7 | 4.58 | 32.445 |
| 8 | 4.64 | 30.515 |
| 9 | 4.58 | 30.444 |
| 10 | 4.72 | 31.239 |
| 11 | 4.98 | 31.448 |
| 12 | 6.08 | 30.67 |
| 13 | 6.16 | 28.305 |
| 14 | 6.42 | 29.979 |
| 15 | 5.64 | 28.305 |

### Example 2. Skin Priming Compositions

A. Skin Priming Composition #1

A skin priming composition was prepared as shown in Table 11.

TABLE 11
Skin priming composition #1

| | % w/w of the |
|---|---|
| Component | Composition |
| water | 86.15 |
| L-arginine | 0.85 |
| diethylene glycol monoethyl | 1.00 |
| ether (e.g., TRANSCUTOL ®) | |
| glycerin | 5.00 |

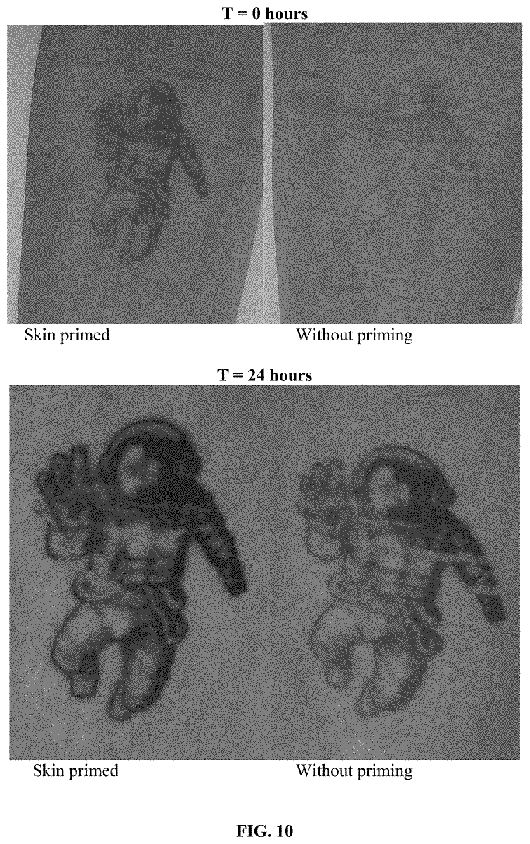

1 mL of the skin priming composition was loaded into a towelette (Diamond Wipe 80 gsm rayon non-woven wipe 7″×3″). The towelette was used to exfoliate, clean, and deposit the skin priming composition onto the semi-permanent tattoo application site. Immediately after the treatment, the semi-permanent tattoo applicator was applied to the treated skin site and removed after 1 hour. FIG. 10 shows the enhanced color density as a result of the skin priming treatment. The color density for the primed skin was comparable to semi-permanent tattoos applied with a heat pad on unprimed skin, both with performances of 8 using the 0-8 color intensity scale (FIG. 11).

B. Skin Priming Composition #2

A skin priming composition was prepared as shown in Table 12.

TABLE 12
Skin priming composition #2

| | % w/w of the |
|---|---|
| Component | Composition |
| water | 94.15 |
| L-arginine | 0.85 |
| glycerin | 5.00 |

The skin priming composition was loaded into a towelette (Diamond Wipe 80 gsm rayon non-woven wipe 7″×3″) at a skin priming composition to wipe mass ratio of 1:2.

B. Skin Priming Composition #3

A skin priming composition was prepared as shown in Table 13.

TABLE 13
Skin priming composition #2

| | % w/w of the |
|---|---|
| Component | Composition |
| Water | 93 |
| L-arginine | 1 |
| Glycerin | 5 |
| Phenoxyethanol and Ethylhexylglycerin | 1 |

The skin priming composition was loaded into a towelette (3″×4″ spunlace 80 gsm 100% rayon with embossed dots) at a skin priming composition to wipe mass ratio of 1:2.

### Example 3. Semi-Permanent Tattoo Applicator Preparation

Materials Preparation

12 inch sections of the unwind side of 3M 1509 adhesive is laminated on the unwind side of Argotec 1.4 mil PU. The laminated section is then cut down to 3″ by 3″ squares. The casting liner of the Argotec 1.4 mil PU is then removed to reveal the polyurethane.

Template Assembly

An Adobe illustrator file of 4 by 4 matrix of semi-permanent tattoo designs of interest are printed onto A4 paper using an Epson DTG printer. Strips of scotch permanent double-sided tape are adhered to the template along the perimeter of the design. The 3″×3″ semi-permanent tattoo blanks ware adhered to the template with the Argotec side down.

Printing

The template is edited to remove the border surrounding the designs. A box is added to the bottom of the template as print quality checker. The template is printed onto the blanks. This is repeated 5 times, with another box added to the template for quality control.

Dehydrating and Final Packaging

The template is placed in the dehydrator set to 60° C. for 90 minutes. The template is removed from the dehydrator and silicone coated liner from the 3M 1509 adhesive is placed onto the printed side. A single liner is removed and placed back on to ensure that there is no formulation transfer. The semi-permanent tattoos are sorted by design packaged in clear polyethylene bags.

### Example 4. Aftercare Composition Preparation

Seven aftercare compositions are prepared as shown in Table 14.

TABLE 14
Aftercare compositions

| | Composition # | | | | | | |
|---|---|---|---|---|---|---|---|
| | 1 | 2 | 3 | 4 | 5 | 6 | 7 |
| Ingredients | w/w % | w/w % | w/w % | w/w % | w/w % | w/w % | w/w % |
| Glycerin | 25% | 25% | 25% | 25% | 25% | 25% | 25% |
| Deionized Water | 36% | 35% | 36% | 36% | 36% | 73% | 63% |
| Triethanolamine | 0.5% | 0.50% | N/A | N/A | N/A | N/A | N/A |
| Acrylates/_{C10-30}Alkyl | 0.3% | 0.30% | 0.30% | 0.30% | 0.30% | 0.30% | 0.30% |
| Acrylate Crosspolymer | | | | | | | |
| Dowsil 7-3110 Volatile | 38% | 38% | 38% | 38% | 38% | N/A | N/A |
| Fluid HIP Emulsion | | | | | | | |
| (Dimethicone, trisiloxane, | | | | | | | |
| ceteth-10, laureth-4) | | | | | | | |
| Preservative | 0.5% | 0.50% | 0.50% | 0.50% | 0.50% | 0.50% | 0.50% |
| L-arginine | N/A | 0.50% | N/A | 0.50% | N/A | 0.50% | 0.50% |
| Panthenol | N/A | N/A | N/A | N/A | 0.50% | 0.50% | 0.50% |
| Sodium Hydroxide | N/A | N/A | * | * | * | * | * |
| Ethanol | N/A | N/A | N/A | N/A | N/A | N/A | 10% |

* pH to 5

### Example 5. Ink Compositions

An ink composition as shown in Table 15 was prepared. The solids (genipin, trehalose, and acid blue 9, e.g., FD&C Blue 1 dye) were weighed out into clean weigh boats. The volatile solvents (water, ethanol, and diethylene glycol monoethyl ether, e.g., TRANSCUTOL®) were then weighed out in separate, clean beakers of volume 500 mL-2 L. The solid genipin was then added to the ethanol and the mixture was stirred either by adding a magnetic stir bar and placing on a magnetic mixer, or by descending the impeller of a mechanical mixer into the solution and mixed at a speed of 400 RPM. The beaker was also covered with PVC plastic wrap to prevent evaporation of the volatile solvent. Trehalose was then added to the beaker containing water and the mixture was stirred either by adding a magnetic stir bar and placing on a magnetic mixer, or by descending the impeller of a mechanical mixer into the solution and mixed at a speed of 400 RPM. The beaker containing diethylene glycol monoethyl ether was then placed on a balance and 1,2-hexanediol (e.g., OPTIPHEN® HD) and phenoxyethanol and ethylhexylglycerin (e.g., EUXYL® PE9010) were added. The mixture was stirred either by adding a magnetic stir bar and placing on a magnetic mixer, or by descending the impeller of a mechanical mixer into the solution and mixed at a speed of 400 RPM. The ethanol solution and the diethylene glycol monoethyl ether (e.g., TRANSCUTOL®) solutions were then added to the water solution. The resultant mixture is then mixed using the mechanical mixer at 400 RPM until a uniform solution was achieved. The resultant mixture was then decanted portion-wise onto a ceramic filter funnel containing a piece of dampened filter paper and the solution was vacuum filtered into the attached vacuum flask. The filtered solution was then decanted into 250 mL or 500 mL plastic bottles sterilized with ethyl alcohol, sealed with screw caps and Parafilm, and placed in a cool, dark cabinet for storage.

TABLE 15
Ink Compositions.

| | Amount |
|---|---|
| Ingredient | (% w/w) |
| Aqua | 30% |
| Ethyl alcohol | 43% |
| diethylene glycol monoethyl ether | 15% |
| (e.g., TRANSCUTOL ® CG) | |
| Genipin (e.g., Genipa plant extract) | 7% |
| Trehalose (e.g., TREHALOSE 100) | 2% |
| Phenoxyethanol and Ethylhexylglycerin | 1% |
| (e.g., EUXYL ® PE9010) | |
| 1,2-Hexanediol (e.g, OPTIPHEN ® HD) | 2% |
| Acid Blue 9 (e.g., FD&C Blue 1) | 0.3% |
| Total | 100% |

### Example 6. Semi-Permanent Tattoo Performance

Longevity and Stability

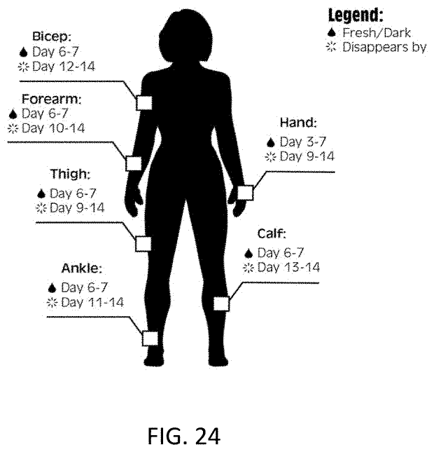

To study the longevity of the ink composition of Example 5, overall aspect, darkness, fading, patchiness and bleeding were evaluated (see Table 16). The study was performed on 20 internal participants at different locations on the body using the ink composition of Example 5 deposited on a combination of an adhesive patch article and an occlusion patch article. The study showed that the general trend for the longevity is 1-2 weeks (see FIG. 24).

A scoring system was set up to assess overall performance of a semi-permanent tattoo using the ink composition of Example 5 once applied on the skin (see Table 16). This assessment was performed daily for a period of 14 days.

TABLE 16
Scoring System to Evaluate Overall Performance

| | |
|---|---|
| Overall | 0 = no visible semi-permanent tattoo |
| performance | 1 = visible semi-permanent tattoo, |
| | unacceptable overall performance |
| | 2 = visible semi-permanent tattoo, |
| | acceptable overall performance |
| | 3 = visible semi-permanent tattoo, |
| | desired overall performance |
| Development | Is the semi-permanent tattoo fully developed? |
| | 0 = no |
| | 1 = yes |
| Patchiness | 0 = no visible alteration |
| score | 1 = slight visible alteration |
| | 2 = moderate, visible deterioration |
| | 3 = marked deterioration, visible deterioration |
| Darkness score | 0 = no visible semi-permanent tattoo |
| | 1 = light (faintly visible ink) |
| | 2 = moderately visible ink |
| | 3 = dark (target ink density) |
| | 4 = very dark (superior to targeted ink darkness) |
| Bleeding score | 0 = no visible bleeding |
| | 1 = slight bleeding |
| | 2 = moderate bleeding |
| | 3 = significant bleeding |
| | 4 = very significant bleeding |
| Fading score | 0 = no fading |
| | 1 = faintly fading |
| | 2 = moderate fading |
| | 3 = significant fading |
| | 4 = no visible tattoo |

The semi-permanent tattoo application requires the use of primer wipes in order to facilitate the development of the dye on the skin. The process for applying the semi-permanent tattoo was as follows:

The primer wipe was used to thoroughly exfoliate the skin using a circular motion for a full 30 seconds at the area the semi-permanent tattoo was to be applied. Wiping with the primer wipe can help smoothen by exfoliation and hydrate skin in preparation for the semi-permanent tattoo. The application area was then allowed to dry.

With the semi-permanent tattoo (e.g., the ink composition of Example 5 deposited on a combination of an adhesive patch article and an occlusion patch article) in hand, the middle portion of the semi-permanent tattoo liner was removed and discarded to expose the clear adhesive and ink composition of Example 5. The sticky side of the adhesive was placed on skin and any air bubbles were gently pressed out. Once the semi-permanent tattoo was placed, the semi-permanent tattoo liner was removed. At this point, the entire semi-permanent tattoo liner was removed from the semi-permanent tattoo applicator. The semi-permanent tattoo was firmly pressed against the skin for 5 seconds to ensure that the adhesive had optimal contact with the skin.

The semi-permanent tattoo was left on the skin for 60 minutes. During this time, the skin should interact with the ink composition of Example 5. Excessive movement and sweating should be avoided to prevent delamination. The semi-permanent tattoo should not be left on for longer than 60 minutes.

The semi-permanent tattoo adhesive was slowly removed to ensure all the ink was transferred to the skin. The semi-permanent tattoo with the ink composition of Example 5 should look blue/green at first and should fully develop into a dark blue or black color over the next 24-36 hours.

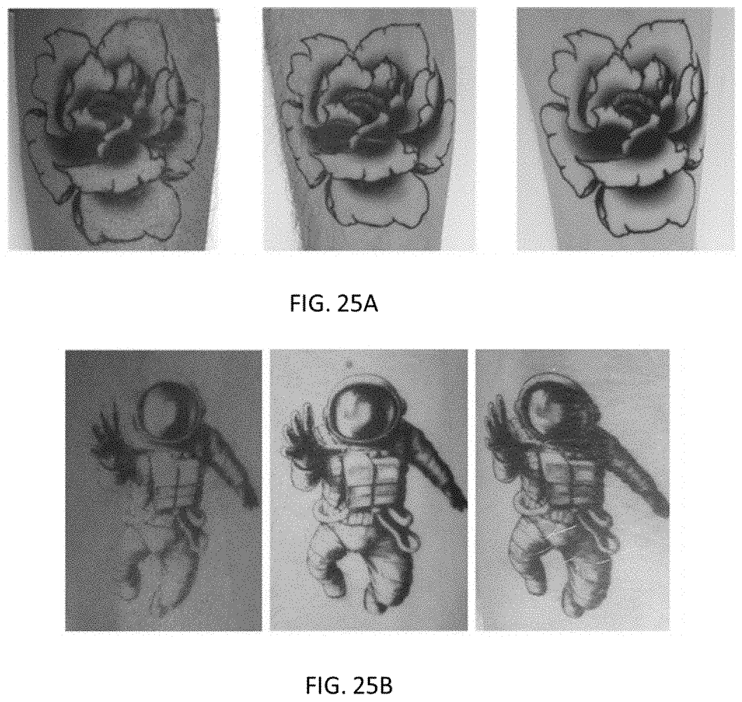

The semi-permanent tattoo performance can be skin tone dependent as the product develops differently in each case (FIGS. 25A and 25B).

In order to evaluate shelf-life, a stability evaluation was performed over the course of 3 months. The performance evaluation (see Table 16) consisted of: (a) applying the semi-permanent tattoo on the subject's forearms; (b) removing the semi-permanent tattoo applicator after 60 minutes; (c) taking a picture post-application; (d) providing recommendations for aftercare; and (e) taking pictures at 24H, 48H up to 14 days post-application. The aftercare recommendations were as follows: moisturize the semi-permanent tattoos with a dime-sized amount of an unscented water-based moisturizer daily (see exemplary ingredient list below); drink plenty of water to keep yourself hydrated; pat semi-permanent tattoos dry with soft towel after showering and do not rub the semi-permanent tattoo; and avoid rubbing the tattoo throughout your daily activities. An example of an water-based moisturizer for daily use can include distilled water (aqua), grapeseed oil (Vitis Vinifera), complete emulsifying wax B, vegetable glycerin, vitamin E oil (tocopherol acetate), GEOGARD™ ECT (e.g., a combination of benzyl alcohol, salicylic acid, glycerin, sorbic acid).

Through repeating the evaluation described above several times, it was determined that these semi-permanent tattoos were able to maintain their initial performance after 10 weeks, i.e., a semi-permanent tattoo that was 10 weeks old was comparably sharp and dark once transferred onto skin compared to a freshly printed semi-permanent tattoo.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modification are within the scope of the following claims.

1. A composition comprising:
one or more semi-permanent colorants;
one or more volatile solvents; and
one or more semi-volatile semi-permanent colorant solubilizers.

2. The composition of claim 1, wherein the composition has a viscosity from about 1 to about 7 centipoise.

3. (canceled)

4. The composition of claim 1, wherein the composition has a surface tension from about 20 to about 35 dynes/cm.

5. The composition of claim 1, wherein the one or more semi-permanent colorant is present in an amount of about 0.1% to about 20% w/w of the composition.

6. (canceled)

7. The composition of claim 1, wherein the one or more semi-permanent colorants are present in an amount of about 5% to about 10% w/w of the composition.

8-9. (canceled)

10. The composition of claim 1, wherein the one or more semi-permanent colorants is genipin.

11-12. (canceled)

13. The composition of claim 1, wherein the one or more volatile solvents are present in an amount of about 60% to about 90% w/w of the composition.

14. The composition of claim 1, wherein the one or more volatile solvents are present in an amount of about 65% to about 85% w/w of the composition.

15-17. (canceled)

18. The composition of claim 1, wherein the one or more volatile solvents are selected from the group consisting of: water, methanol, ethanol, isopropanol, and a combination of two or more thereof.

19. The composition of claim 1, wherein the one or more volatile solvents are a combination of water and ethanol.

20-21. (canceled)

22. The composition of claim 1, wherein the one or more semi-volatile semi-permanent colorant solubilizers are present in an amount of about 0.1% to about 25% w/w of the composition.

23-24. (canceled)

25. The composition of claim 1, wherein the one or more semi-volatile semi-permanent colorant solubilizers are selected from the group consisting of: a polyethylene glycol, an alkyl glycol, and an alkylene glycol ether.

26. (canceled)

27. The composition of claim 1, wherein the one or more semi-volatile semi-permanent colorant solubilizers is diethylene glycol monoethyl ether.

28. The composition of claim 1, wherein the composition further comprises one or more surface-active agents.

29-34. (canceled)

35. The composition of claim 1, wherein the composition further comprises one or more matrix components.

36-43. (canceled)

44. The composition of claim 1, wherein the composition further comprises one or more skin penetration enhancers.

45-48. (canceled)

49. The composition of claim 1, wherein the composition further comprises one or more temporary colorants.

50-60. (canceled)

61. The composition of claim 1, wherein the composition comprises:
purified genipin present in an amount of about 5% to about 10% w/w of the composition;
one or more volatile solvents present in an amount of about 70% to about 85% w/w of the composition, wherein the one or more semi-volatile solvents are water and ethanol;
one or more semi-volatile semi-permanent colorant solubilizers present in an amount of about 10% to about 20% w/w of the composition, wherein the one or more semi-volatile semi-permanent colorant solubilizers is diethylene glycol monoethyl ether;
one or more surface-active agents present in an amount of about 0.25% to about 5% w/w of the composition; and
one or more matrix components present in an amount of about 0.1% to about 5% w/w of the composition.

62-74. (canceled)

75. A skin priming composition comprising:
one or more volatile solvents;
one or more amines; and
one or more humectants.

76-93. (canceled)

94. An adhesive applicator article comprising:
a composition according to claim 1;
an adhesive; and
an adhesive backing.

95-101. (canceled)

102. An adhesive applicator article comprising:
a composition according to claim 1;
a first adhesive layer;
a first backing;
a second adhesive layer; and
a second backing.

103-116. (canceled)

117. An adhesive applicator article, comprising:
a liner;
an adhesive layer to couple to the liner;
a stack up to couple to the adhesive layer;
a removable liner to contact the stack up; and
a border to support the stack up.

118-126. (canceled)

127. An adhesive applicator article, comprising:
an external layer positioned over a plurality of panels;
one or more tabs coupled to the plurality of panels; and
an end portion of the external layer to receive the one or more tabs.

128-131. (canceled)

132. An adhesive applicator article, comprising:
an external layer positioned over a composition according to claim 1;
a first portion of the external layer and a second portion of the external layer; and
a first side of the first portion and a second side of the first portion.

133-144. (canceled)
